(19) United States
(12) Patent Application Publication
(10) Pub. No.: US 2005/0182047 A1
(43) Pub. Date: Aug. 18, 2005

(54) Treatment of demyelinating disorders

(75) Inventors: Lechoslaw Turski, Berlin (DE); Terence Smith, Cambridge (GB)

Correspondence Address: WILMER CUTLER PICKERING HALE AND DORR LLP, 60 STATE STREET, BOSTON, MA, 02109, US

(73) Assignee: Eisai Co., Ltd., Tokyo (JP)

(21) Appl. No.: 11/043,732

(22) Filed: Jan. 26, 2005

Related U.S. Application Data
(62) Division of application No. 09/746,662, filed on Dec. 22, 2000, now abandoned.
Application No. 09/746,662 is a continuation of application No. PCT/GB99/02112, filed on Jul. 2, 1999.

(30) Foreign Application Priority Data
Nov. 6, 1998 (GB) GB 9824393
Jul. 2, 1998 (GB) GB 9814380

(51) Int. Cl. A61K 31/5513

(52) U.S. Cl. 514/220; 514/221

(57) ABSTRACT

This invention is directed to pharmaceutical compositions and methods for treating demyelinating disorders based upon inhibitors of the interaction of glutamate with the AMPA and of the interaction of glutamate with the kainite receptor complex.

This application is a divisional application of U.S. patent application Ser. No. 09/746,662, filed Dec. 22, 2000, which is a continuation application of PCT/GB99/02112, filed Jul. 2, 1999, claiming priority to GB 9814380.3 and GB 9824393.4, filed Jul. 2, 1998 and Nov. 6, 1998, respectively in Great Britain.

The present invention relates inter alia to the treatment of demyelinating disorders.

The majority of excitatory synaptic responses in mammalian CNS are elicited by amino acids such as L-glutamate or L-aspartate and are mediated by four different receptor subtypes. Three of these receptors are coupled to ionophores and are known as the N-methyl-D-aspartate (NMDA), the AMPA α-amino-3-hydroxy-5-methyl-4-isoxazole-propionate), and the kainite receptors. Typically these receptors will be in the form of a receptor complex including for example glutamate and/or agonist binding site(s), modulatory site(s) and an ion channel, and possibly also including other moieties interacting with the function of the channel. The fourth receptor subtype is linked to phosphoinositol metabolism and is known as the metabotropic glutamate receptor.

The NMDA receptor is coupled to high conductance channels permeable to NA⁺, K⁺, and Ca²⁺ (McBain C J, Mayer M (1994): N-Methyl-D-aspartic acid receptor structure and function, Physiol. Rev., 74:723-760). It is modulated by glycine (coagonist) and polyamines (positive modulator) and is blocked in a use and voltage dependent manner by Mg²⁺. The functional NMDA receptor is thought to be formed as a pentameric subunit assembly consisting of subunit selection from NR1 (eight isoforms) and NR2 (four isoforms) families (Hollmann M, Heinemann S (1994): Cloned glutamate receptors, Annu. Rev. Neurosci. 17:31-108). The type of subunits forming the NMDA channel determine its biophysical properties and physiological function (Schöpfer R, Monyer H, Sommer B, Wisden W, Sprengel R, Kuner T, Lomeli H, Herb A, Kohler M, Burnashev N (1994): Molecular biology of glutamate receptors, Prog. Neurobiol. 42:353-357). The AMPA and kainite receptors are permeable to Na⁺ and K⁺ (Hollmann and Heinemann, 1994 [supra]). AMPA receptor-dependent ion channel is formed from four different subunits designated as GluR1 to GluR4 (in two alternative splice variants—flip and flop) in a tetrameric subunit assembly (Hollmann and Heinemann, 1994 [supra]; Rosenmund C, Stern-Bach Y, Stevens C (1998): The tetrameric structure of a glutamate receptor channel, Science 280:1596-1599). Pharmacological properties of AMPA receptor-dependent ion channels are determined by the selection of subunits. Channel assemblies lacking GluR2 subunits are permeable to Ca²⁺ in addition to Na⁺- and K⁺-permeability (Hollmann and Heinemann, 1994 [supra]). In situ hybridization has revealed different expression of glutamate receptor subunits throughout the brain and during development (Monyer H, Burnashev N, Laurie D J, Sakmann B, Seeburg P (1994): Developmental and regional expression in the rat brain and functional properties of four NMDA receptors, Neuron 12:529-540).

Kainate receptors represent the third type of ionotropic glutamate receptors (E. A. Barnard, Ionotropic glutamate receptors: new types and new concepts. Trends Pharmacol. Sci. 18: 141-148, 1997). The kainate receptors are formed heterometrically by GluR5-7 and KA1-2 types of subunits (Y. Paas, The macro- and microarchitectures of the ligand-binding domain of glutamate receptors. Trends in Neurosci. 21, 117-125, 1998). By being activated (opened) and desensitized (closed) by glutamate, kainate receptors modulate a passive flow of Na+, K+ and to varying degree, Ca2+ ions across the cell membrane. As such kainate receptors mediate fast synaptic transmission in the nervous system and are involved in plasticity, transmission of sensory signals and in development (E. A. Barnard ibid). Furthermore, kainate receptors are unevenly distributed in the brain and spinal cord of rodents and primates (J. M. Henley, Trends Pharmacol. Sci. 15, 182-190, 1994). Dysfunction of kainate receptors may contribute to pathogenesis of variety of neurological and psychiatric disorders (B. Meldrum and J. Garthwaite, Trends Pharmacol. Sci. 11, 379-387, 1990).

In contrast to the well documented role of glutamate in the pathogenesis of neuronal degeneration resulting from hypoxia/ischemia, hypoglycemia, convulsions and head or spinal cord trauma, no clear link has been established between glutamate-mediated cell death and demyelinating disorders. Many demyelinating disorders have previously been resistant to therapy. Furthermore, until recently, the treatment of human demyelinating disorders has relied exclusively on the use of immunosupressive agents such as corticosteroids and cyclophosphamide, which although providing limited benefit to patients, can be associated with potentially serious side effects. The introduction of interferon preparations has provided efficacy in the treatment of certain demyelinating disorders (e.g. multiple sclerosis). However, as benefits are apparent in only a portion of the subgroup of patients classified as suitable for tratment, then management of the disease is still insufficient with such preparations.

The present inventors have now provided evidence in support of the involvement of glutamate in the pathogenesis of demyelinating disorders. They have established a link between neuronal demyelination and glutamate-mediated cell death using accepted animal models of a demyelinating disorder.

The present invention represents a major advance over prior art methods in the treatment of demyelinating disorders.

According to one aspect of the present invention, there is provided the use of an inhibitor of the interaction of glutamate with the AMPA and/or kainate receptor complex in the manufacture of a medicament for treating a demyelinating disorder.

The term “inhibitor of the interaction of glutamate with the ADA and/or kainate receptor complex” is used herein to include moieties that bind to the AMPA and/or kainate receptor or to glutamate so as to prevent or reduce the binding of glutamate to its binding site on the AMPA and/or kainate receptor. Such moieties may bind in a competitive or non-competitive manner. They are referred to herein as “antagonists” of the binding of glutamate to the AMPA and/or kainate receptor. A skilled person is able to identify substances that may be useful as antagonists of the present invention by binding studies. For example, the AMPA and/or kainate receptor, a part thereof including said glutamate binding site, or a glutamate molecule can be used to screen for substances that bind thereto, preferably in a highly specific manner. Such binding studies can be part of a screening program for identifying or designing potential therapeutic agents. More specifically, a skilled person could identify inhibitors of the interaction of glutamate with the AMPA and/or kainate receptor complex using, for example, in vitro calcium ion-increase assays or the whole cell configuration of the patch clamp technique. Cells expressing the AMPA receptor complex could be obtained, for example, from dissociated cortical or hippocampal cells. Cells expressing the kainate receptor complex could be obtained, for example, from dissociated dorsal root ganglion cells. Inhibition of the interaction of agonists, for example glutamate, AMPA or kainate, of the AMPA and/or kainate receptor complex could be assayed by incubation of the agonist with and without antagonist and the cellular response (eg change in intra-cellular calcium ion concentration or change in membrane potential) measured.

The term “inhibitor of the interaction of glutamate with the AMPA and/or kainate receptor complex” also includes moieties that prevent a signal being transmitted that would otherwise occur when glutamate binds to the AMPA and/or kainate receptor. Preferred such moieties are AMPA and/or kainate receptor channel blockers. The term “AMPA and/or kainate receptor channel blocker” is used herein to refer to moieties that reduce the permeability of ion channels associated with the AMPA and/or kainate receptor in vivo (preferably to Na⁺,K⁺ and/or Ca²⁺ ions).

Various antagonists and AMPA receptor channel blockers that are within the scope of the present invention will now be described in greater detail:

Antagonists

The antagonists of the present invention include L-glutamate derivatives such as e.g. L-glutamic acid diethylester, α-amino-3-hydroxy-5-methyl-4-isoxazolepropionate derivatives such e.g., a-amino-3-hydroxy-5-tert-buthyl-4-isoxazolepropionic acid, quinoline, quinoxaline, quinoxalinedione, quinazolinone, phenylpyridazino-indole-1,4-dione, indeno-pyrazinone, indeno-pyrazine-carboxylic acid, indolo-pyrazinone, imidazo-pyrazinone, amino-phenyl-acetic acid, benzothiadiazine, 4-hydroxypyrrolone, 4-hydroxy-pyrrolo-pyridazinone, quinolone, amino alkanoic acid, isatin, nitroquinolone, phenyl-azolophthalazine, amino- or desamino- 2,3-benzodiazepine, 2,3-benzodiazepin-4-one, β-carboline-3-carboxylic acid, alkoxy-phenyl-benzodiazepine, acetyl-aminophenyl-dihydro-methyl-dioxolo-benzodiazepine, oxadiazol, isatinoxime, decahydroisoquinoline, and sulphamate.

Further substances that may be useful as antagonists are listed below:

### List of Antagonists

(1) ω-[2-(Phosphonoalkyl)phenyl]-2-aminoalkanoic acids (I) in WO 93-05772 as shown below:

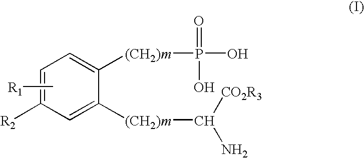

ω-[2-(Phosphonoalkyl)phenyl]-2-aminoalkanoic acids represented by formula (I), wherein n and m independently are 0, 1, 2 or 3; R¹ is selected from the group consisting of hydrogen and R²; R² is selected from the group consisting of hydrogen, halogen, halomethyl, nitro, amino, alkoxy, hydroxyl, hydroxymethyl, C₁ to C₆ lower alkyl, C₇ to C₁₂ higher alkyl, aryl and aralkyl, wherein if R² is hydrogen, R¹ is not hydrogen; R³ is selected from the group consisting of hydrogen and C₁ to C₆ lower alkyl; the stereoisomers thereof in their resolved or racemic form, and pharmaceutically acceptable salts thereof.

(2) Fused pyperazine derivatives in WO 92-07847 as shown below:

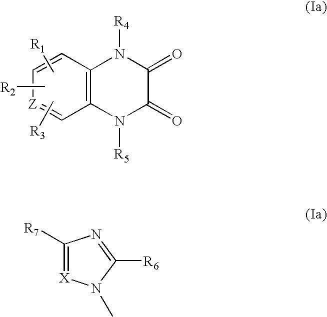

A pyperazine derivative represented by general formula (Ia) wherein Z represents C or N, provided that two Zs are not N atoms at the same time; R¹ represents (1a) wherein X represents N or R⁸C, R⁶ represents H or alkyl, and R⁷ and R⁸ represent each H, alkyl, nitro or phenyl, or alternatively R⁷ and R⁸ are combined together to represent butadienylene or 1,4-butylene; R² and R³ represent each H, F, cyano, acyl, nitro, alkyl, morpholino or R¹; R⁴ and R⁵ represent each H, hydroxy, alkyl, cycloalkyl, heterocycle, phenyl or Y-substituted alkyl; Y represents hydroxy, acyloxy, F-substituted methyl, cycloalkyl, tetrahydrofuryl, carboxyl, alkoxy carbonyl or NR⁹R¹⁰; and R⁹ and R¹⁰ represent H or alkyl, or alternatively R⁹ and R¹⁰ are combined together to represent a 5- or 6-membered cyclic group which may contain oxygen atom(s).

(3) Triazoloquinoxalin-1,4-diones (I) and (II) in WO 93-06103 an shown below:

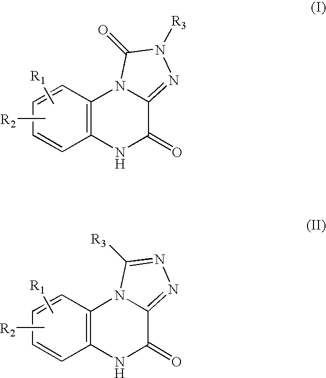

Quinoxaline compounds represented by formula (I) or (II), wherein R¹ and R² are independently hydrogen, C₁₋₆-alkyl, halogen, NO₂, NH₂, CN, CF₃, SO₂NR⁴R⁵ wherein R⁴ and R⁵ are independently hydrogen or C₁₋₆-alkyl, or COR⁶ wherein R⁶ is C₁₋₆-alkyl; and R³ is hydrogen, C₁₋₆-alkyl or CF₃, and compositions thereof.

(4) [1,2,4]Triazolo[4,3-a]quinoxalinone derivatives (I) in WO 96-08493 A1 as shown below:

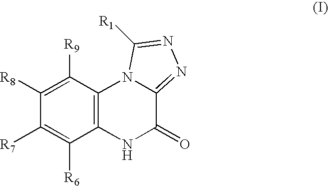

[1,2,4]triazolo[4,3-a]quinoxalinone compounds of general formula (I) wherein R¹ is POX′X″ or alkyl substituted with COX′ or POX′X″, and X′ and X″ independently are hydroxy or alkoxy, and R⁶, R⁷, R⁸ and R⁹ independently are hydrogen; alkyl; halogen; NH₂; NO₂; CN; CF₃ SO₂NY′Y″ OR COZ′ wherein Z′ is NY′Y″ or alkyl and Y′ and Y″ independently are hydrogen or alkyl; triazolyl; imidazolyl substituted with phenyl or alkyl.

(5) [1,2,4]Triazolo[4,3-a]quinoxalinone derivatives (I) in WO 96-08492 A1 as shown below:

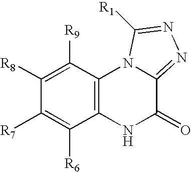

[1,2,4]triazolo[4,3-a]quinoxalinone compounds of general formula (I) wherein R¹ is POX′X″ or alkyl substituted with COX′ or POX′X″ and X′ and X″ independently are hydroxy or alkoxy, and R⁶, R⁷, R⁸ and R⁹ independently are hydrogen; alkyl; halogen; NH₂; NO₂, CN, CF₃, SO₂NY′—Y″, COZ′ wherein Z′ is NY′Y″ or alkyl and Y′ and Y″ independently are hydrogen or alkyl; triazolyl; imidazolyl, piperidino, piperazinyl, morpholino or thiomorpholino; all rings optionally being substituted.

(6) Pyrrolylquinoxalindiones (I) in WO 97 49701 as shown below:

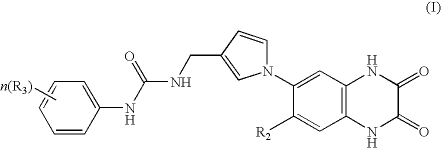

Pyrrolylquinoxalindiones of formula (I) and their tautomeric and isomeric forms and their physiologically acceptable salts, in which R¹ is hydrogen, C₁-C₆ alkyl, substituted by hydroxyl or carboxyl, R₂ is hydrogen, C₁-C₆ alkyl, C₂-C₆ alkenyl, C₂-C₆ alkynyl, a chlorine, fluorine or bromine atom, a trihalogen methyl, cyano, or nitro group or SO₂C₁C₄ alkyl, R³ is COOH or a radical hydrolysable to form the carboxyl group, and n is 1 or2.

(7) Imidazole-substituted quinoxalinedione derivatives (I) in WO 97-46555 as shown below:

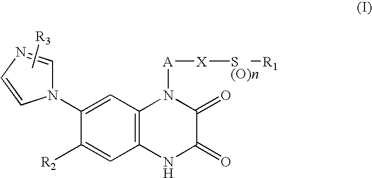

Substituted imidazole quinoxalinedione derivatives represented by general formula (I), wherein each symbol has the following meaning: A: (CH_{?}2_{?})ₘ or Ph-(CH₂)ₚ (Ph being phenyl); X: oxygen or NR⁴; R¹: hydrogen, hydroxy or triazolyl, provided that X may be a bond when R¹ is triazolyl; R²: hydrogen, nitro, halogenated lower alkyl, cyano, amino, mono- or di (lower alkyl)amino, or halogeno; R³ and R⁴; the same or different and each representing hydrogen or lower alkyl; n: 0, 1 or 2; m: an integer of 2 to 6; and p: an integer of 1 to 6.

(8) Heterocyclically substituted imidazoloquinoxalines (I) in WO 97-34896 as shown below:

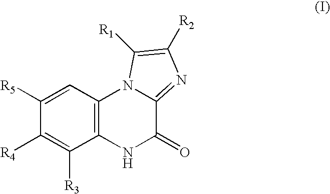

Imidazoloquinoxalines of formula (I), wherein R¹ to R⁴ have the meanings given in the description in the corresponding patent (WO 97-34896) and R⁵ is a five-member optionally substituted heterocycle with between 1 and 4 nitrogen atoms or with 1 or 2 nitrogen atoms and an oxygen or sulphur atom, or an R⁶-substituted phenyl ring.

(9) Quinoxaline derivatives (I) in WO 97-32858 as shown below:

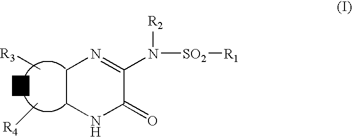

Quinoxaline derivatives of the formula (I) wherein R¹ is alkyl, halo (lower) alkyl, amino, aryl or heterocyclic group, R² is hydrogen or lower alkyl, R³ and R⁴ are each independently hydrogen, cyano, nitro, halogen, lower alkyl, halo (lower) alkyl, lower alkoxy, halo (lower) alkoxy, di (lower) -alkylamino, aryl which may have one or more substituents (s), heterocyclic group which may have one or more substituents (s), lower alkylthio which may have one or more substituents (s), heterocyclicthio, lower alkylsulfonyl, lower alkylaminosulfonyl, or heterocyclicsulfonyl, a group of the formula:

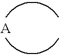

A is the group of the formula:

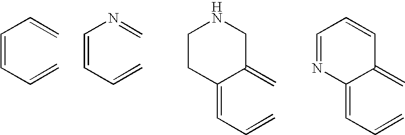

It is to be noted the object compound (I) may include one or more stereoisomers due to asymmetric carbon atom (s) and double bond, and all of such isomers and a mixture thereof are included. It is further to be noted that isomerization or rearrangement of the object compound (I) may occur due to the effect of the light, acid, base or the like, and the compound obtained as the result of said isomerization or rearrangement is also included within the scope of the present invention. It is also to be noted that the solvating form of the compound (I) (e.g. hydrate, etc.) and any form of the crystal of the compound (I) are included within the scope of the present invention.

(10) Condensed 2,3-benzodiazepine derivatives (I) in WO 97-28163 as shown below:

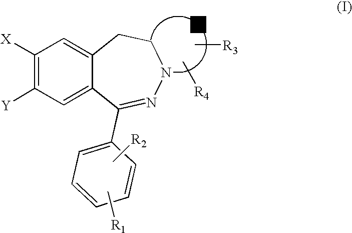

2,3-Benzodiazepine derivatives of the formula (I) wherein R¹ and R² are identical or different and hydrogen, C₁-C₆-alkyl, nitro, halogen, cyano, the group —NR⁸R⁹, —O—C₁₋₄-alkyl, —CF₃, OH or C₁₋₆-alkanoyloxy; R³ and R⁴ are identical or different and hydrogen, halogen, C₁-C₆-alkoxy, hydroxy, thiocyanate, C₁-C₆-alkylthio, cyano, COOR¹², PO₃R¹³R¹⁴, C₁-C₆-alkanoyl, C₁-C₆-alkanoyloxy, eventually with C₁-C₄-alkoxy or phenyl-substituted C₂₋₆-alkynyl, eventually with C₁₋₄-alkoxy or phenyl-substituted C₂₋₆-alkenyl, eventually with halogen, hydroxy, C₁-C₆-alkoxy, C₁-C₆-thioalkyl, NR¹⁰—R¹¹-substituted C₁-C₆-alkyl, C₃₋₇-cycloalkyl or eventually a substituted aryl- or hetaryl-rest; R⁸ and R⁹ are identical or different and hydrogen, C₁-C₆-alkyl or the group-CO—C1-6-alkyl; R¹⁰ and R¹¹ are identical or different and hydrogen, C₁-C₆-alkyl or C₁₋₆-alkanoyl or together with the nitrogen atom will bild a 5-7 branched saturated heterocyclus, which will contain and can be substituted with a further oxygen-, sulfur or nitrogen atom; R¹², R¹³, R¹⁴ are identical or differnt and H or C₁-C₆-alkyl; X hydrogen or halogen; Y C₁₋₆-alkoxy or X and Y together —O—(CH2)n-O—; n means 1,2 or 3 and A together with the nitrogen will form a saturated or an unsaturated 5 armed heterocyclus, which can contain 1-3 nitrogen atoms and/or a oxygen atom and/or one or two carbonyl groups or their isomers or physiological salts thereof.

(11) 1,2,3,4-Tetrahydroquinoxalindione derivatives (I) in WO 96-10023 as shown below:

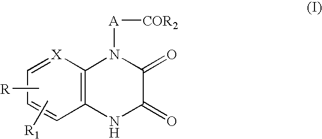

A 1,2,3,4-tetrahydroquinoxalindione derivative represented by general formula (I) or a salt thereof, an NMDA-glycine receptor and/or AMPA receptor antagonist and a kainate neurocytotoxicity inhibitor each containing the same, and a medicinal composition comprising the above-mentioned compound and pharmaceutically acceptable carriers: wherein X represents N or CH; R represents imidazolyl or di(lower alkyl)amino; R¹ represents (I) halogeno, nitro, cyano, carboxy, amino, mono- or di(lower alkyl) amino, lower alkanoyl, lower alkythio, lower alkylsulfinyl, lower alkylsulfonyl, or carbamoyl, (2) lower alkyl or lower alkoxy which may be substituted by halogeno, carboxy or aryl, or (3) phenyloxy which may be substituted by lower alkoxycarbonyl or carboxy; R² represents hydroxy, lower alkoxy, amino, or mono- or di(lower alkyl)amino; and A represents optionally substituted alkylene or —O—B— (B being lower alkylene); provided the case wherein R represents imidazolyl, R¹ represents cyano, A represents ethylene and R² represents hydroxy is excepted.

(12) New heterocyclic substituted imidazoloquinoxalinones (I) in WO 96-10572 as shown below:

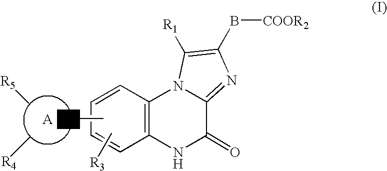

Imidazoloquinoxalinones of the formula (I), in which R¹ stands for hydrogen, branched or linear C₁₋₅-alkyl or a phenyl, pyridyl or thienyl group possibly substituted by one to two chlorine atoms, a trifluoromethyl, a nitrodioxy or a methylene dioxy group; R² stands for hydrogen, C₁₋₅-alkyl or C₃₋₈-dialkylaminoalkyl; R³ stands for a chlorine or bromine atom, a trifluoromethyl, cyano or nitro group; A stands for a five-membered heterocycle with 1-4 nitrogen atoms or 1-2 nitrogen atoms and one oxygen or sulphur atom possible substituted by R⁴ and R⁵; the radicals R⁴ and R⁵, that may be the same or different, stand for hydrogen, C₁₋₅-alkyl, C₁₋₅-hydroxyethyl, phenyl, phenyl substituted by a chlorine atom, a trifluoromethyl or nitro group, —CHO, —COOH, —COO—C₁₋₅-alkyl, —CH₂—NR⁶R⁷ (in which R⁶═H, C₁₋₅-alkyl, R⁷═H, C₁₋₅-alkyl), —CH₂—NH—CO—R⁸ (in which R⁸═C₁₋₅-alkyl, phenyl, a phenyl group or an heteroaryl group possibly substituted by a chlorine atom of a nitro or trifluoromethyl group) or —CH₂—NHCONHR⁸, and B stands for a bond or a C₁₋₅-alkylene chain. Also disclosed are the tautomer and isomer forms of these compounds, as well as their physiologically compatible salts.

(13) Fused indole and quinoxaline derivatives (I) in WO 9608495 A1 as shown below:

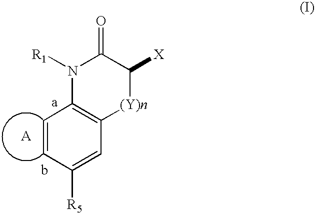

Compounds having formula (I) or a pharmaceutically acceptable salt thereof wherein: R¹ is hydrogen, alkyl or benzyl; X is O or NOR², wherein R² is hydrogen, alkyl or benzyl; Y is N—R⁴ wherein R⁴ is hydrogen, OH or alkyl; n is 0 or 1; R⁶ is phenyl which is substituted one or more times with substituents selected from the group consisting of SO₂NR′R″, CONR′R″, and COR′″ wherein R′ and R″ each independently are hydrogen, alkyl, or —(CH₂)ₚ—W, wherein p is 0, 1, 2, 3, 4, 5, or 6, and W is hydroxy, amino, alkoxycarbonyl, or phenyl which may be substituted one or more times with substituents selected from the group consisting or halogen, CF₃, NO₂, amino, alkyl, alkoxy or methylenedioxy; or wherein R′ and R″ together are (CH₂),Z(CH₂), wherein r and s each independently are 0, 1, 2, 3, 4, 5 or 6 and Z is O, S, CH₂ or NR″″ wherein R″″ is hydrogen, alkyl, or —(CH₂)ₚ—W, wherein p is 0, 1, 2, 3, 4, 5 or 6, and W is hydroxy, amino, alkoxycarbonyl, or phenyl which may be substituted one or more times with substituents selected from the group consisting of halogen, CF₃, NO₂, amino, alkyl, alkoxy or methylenedioxy; and wherein R′″ is hydrogen, alkyl, alkoxy or phenyl which may be substituted one or more times with substituents selected from the group consisting of halogen, CF₃, NO₂, amino, alkyl, alkoxy or methylenedioxy; A is a ring of five to seven atoms fused with the benzo ring at the positions marked a and b.

(14) [1,2,4]Triazolo[4,3-a]quinoxaline compounds (1) in WO 94-26746 as shown in below:

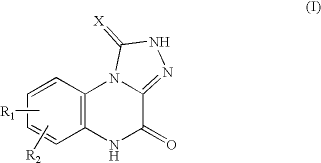

[1,2,4]Triazolo[4,3-a]quinoxaline derivatives of general formula (I) wherein one of R¹ and R² is a 5- or 6-membered N-containing heterocyclic ring optionally substituted, or a fused ring system comprising a 5- or 6-membered N-containing heterocyclic ring optionally substituted; and the other of R¹ and R² is H, alkyl, alkoxy, halogen, NO₂, NH₂, CN, CF₃, COC₁₋₆-alkyl or SO₂NR′R″, wherein R′ and R″ are independently H or alkyl and X is O or S; and pharmaceutically acceptable salts thereof.

(15) [1,2,4]Triazolo[4,3-a]quinoxaline derivatives (I) in WO 94-21639 as shown below:

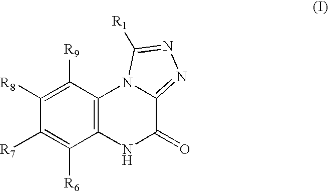

Quinoxaline compounds of general formula (I) wherein R1 is COX′, POX′X″ or alkyl substituted with COX″ OR POX′X″, and X′ and X″ independently are hydroxy or alkoxy, and R⁶, R⁷, R⁸ and R⁹ independently are hydrogen, alkyl, halogen, NH₂, NO₂, CN, CF₃, SO₂NY′Y″ or COZ′ wherein Z′ is NY′Y″ or alkyl and Y′ and Y″ independently are hydrogen or alkyl, triazolyl, imidazolyl, imidazolyl substituted with phenyl or alkyl, or R⁶ and R⁷, or R⁸ and R⁹, together form a further fused ring.

(16) 2H-1,2,4-Benzothiadiazine-1,1-dioxide-3-carboxylic acid derivatives (I) WO 93-21171 as shown below:

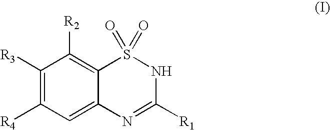

The present invention relates to the use of derivatives of the 2H-1,2,4-benzothiadiazin-1,1-dioxide-3-carboxylic acid of the above formula or the salts of such compound or of intermediates of such compound for the preparation of AMPA receptor antagonists and to new componds of the formula (I), their preparation and the medications in which they are found.

In the formula (I): R₁ is carboxy, alkoxycarbonyl, tetrazolyl, —CO—NH₂, —CO—NH-alk, —CO—N(alk)₂, —CO—NHOH, —CO—N(alk)OH, —CO—NH—O—R₅, —CO—N(alk)—OR₅ or a group that may be converted into a carboxyl moiety in vivo; R₂, R₃ and R₄ are the same or different and are selected from the group consisting of hydrogen, halogen or alkyl; R₅ is alkyl or phenylalkyl.

The term alk refers to an alkyl or alkylene group. Clearly, the compounds of the present invention include the tautomers of the compounds of the formula (I). The groups, convertible into carboxyl moieties in vivo, include —CO—R₆, in which R₆ is O-alk-R₇, —O-alk-O—CO-alk, —O-alk-O—COOalk, —O-alk-O—CO—R₇, —O-alk-OH, —O-alk-O-alk, —O-alk-S-alk, —O-alk-O—R₇, —O-alk-S—R₇, —O-alk-COOH, —O-alk-COOalk, —O-alk-NR₈R₉, —NH-alk-O—CO-alk, —NH-alk-O—COOalk, —NH-alk-O—CO—R₇, —NH-alk-OH, —NH-alk-O-alk, —NH-alk-S-alk, —NH-alk-O—R₇, —NH-alk-S—R₇, —NH-alk-COOH, —NH-alk-COOalk, —NH-alk-NR₈R₉. In these definitions, R is alkyl or alkylene, R₇ phenyl, R₈ and R₉ are the same or different and are selected from the group consisting of hydrogen, alkyl, phenyl or phenylalkyl or form with the oxygen atom they are attached to a piperidinyl, morpholinyl or pyrrolidinyl ring. The halogen atoms are selected from the following: fluoride, chloride, bromide or iodide. Unless otherwise stated, in the above and below definitions, the alkyl, alkoxy and alkylene groups are a straight or branched alkyl chain having one to six carbon atom, and preferably one to four carbon atoms. The compounds of the formula (I) in which either R₂, R₃ and R₄ are hydrogen and R₁ is carboxy, alkoxycarbonyl, —CO—NH₂ or —CO—NH-alk, or R₄ a chloride or bromide atom, R₂ and R₃ are hydrogen and R₁ is carboxy, alkoxycarbonyl, —CO—NH₂ or —CO—NH-alk, or R₃ a chloride or bromide atom, R₂ and R₄ are hydrogen and R₁ is carboxy, alkoxycarbonyl, —CO—NH₂ or —CO—NH-alk. The present invention include also other compounds of the formula (I), their salts or intermediates of their salts. In these compounds, R₁ is carboxy, alokoxycarbonyl, tetrazolyl, —CO—NH₂, —CO—NH-alk, —CO—N(alk)₂, —CO—NHOH, —CO—N(alk)OH, CO—NH—O—R₅, CO—N(alk)-OR₅ or —CO—R₆, in which R₆ is —O-alk-R₇, —O-alk-O—CO-alk, —O-alk-O—COOalk, —O-alk-O—CO—R₇, —O-alk-OH, —O-alk-O-alk, —O-alk-S-alk, —O-alk-O—R₇, —O-alk-S—R₇, —O-alk-COOH, —O-alk-COOalk, —O-alk-NR₉, —NH-alk-O—CO-alk, —NH-alk-O—COOalk, —NH-alk-O—CO—R₇, —NH-alk-OH, —NH-alk-O-alk, —NH-alk-S-alk, —NH-alk-O—R₇, —NH-alk-S—R₇, —NH-alk-COOH, —NH-alk-COOalk, —NH-alk-NR₈R₉, R₂, R₃ and R₄ are the same or different and are selected from the group consisting of hydrogen, halogen or alkyl, R₅ is alkyl or phenylalkyl, R₇ is phenylalkyl, R₈ and R₉ are the same or different and are selected from the group consisting of hydrogen, alkyl, phenyl or phenylalkyl or form with the oxygen atom they are attached to a piperidinyl, morpholinyl or pyrrolidinyl ring. The term alk refers to an alkyl or alkylene group. The present invention does not include the compounds of the formula (I) in which either R₂, R₃ and R₄ are hydrogen and R₁ is carboxy, alkoxycarbonyl, —CO—NH₂ or —CO—NH-alk, or R₄ a chloride or bromide atom, R₂ and R₃ are hydrogen and R₁ is carboxy, alkoxycarbonyl, —CO—NH₂ or —CO—NH-alk, or R₃ a chloride or bromide atom, R₂ and R₄ are hydrogen and R₁ is carboxy, alkoxycarbonyl, —CO—NH₂ or —CO—NH-alk.

(17) Fused quinoxalinone derivatives (I) in WO 93-20077 as shown in below:

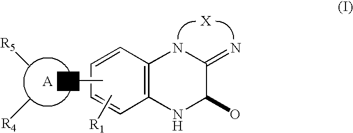

A fused quinoxalinone derivative represented by general formula(I), a tautomeric isomer thereof, a pharmaceutically acceptable salt thereof, or a pharmaceutical composition containing the same, which has a glutamate receptor antagonism and is useful as anti-ischemic and pshychotropic, wherein a represents a 5-membered heterocyclic group containing two or three nitrogen atoms, R¹ represents nitro or trifluoromethyl, X represents (a), (b), (c) or (d), and R², R³, R⁴, R⁵ and R⁶ may be the same or different from one another and each represents hydrogen or lower alkyl which may be substituted by mono- or di(lower alkyl)amino.

(18) Quinolone derivatives (I) in WO 93-11115 as shown in below:

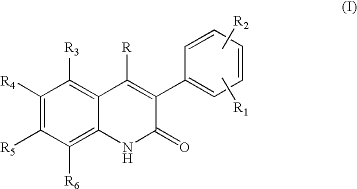

Compounds of formula I or a pharmaceutically acceptable salt thereof or a prodrug thereof: wherein R represents a hydrogen atom, an amino group, a carboxy or C₂₋₆ alkoxycarbonyl group, or a group of formula -A-B-E, in which A represents a chemical bond, an oxygen or sulphur atom, or an —NH— group; B represents a carbonyl (C═O) or sulphonyl (SO₂) group, or a straight or branched alkylene chain containing from 1 to 6 carbon atoms; and E represents C₁₋₆ alkyl, C₂₋₆ alkenyl, C₂₋₆ alkynyl, cyano, phenyl, tetrazolyl, methyloxadoazolyl, —NR^{a}R^{b}, —COR^{a}, —C(═N.OR^{a})R^{b}, —CO₂R^{a}, —CONR^{a}R^{b}, —CONR^{a}.OR^{b} or —CH₂CO₂R^{a}; R¹ and R² independently represent hydrogen, hydrocarbon, a heterocyclic group, halogen, cyano, trifluoromethyl, nitro, —OR^{a}, —SR^{a}, —SOR^{a}, —SO₂R^{a}, —SO₂NR^{a}R^{b}, —NR^{a}R^{b}, —NR^{a}COR^{b}, —NR^{a}CO₂R^{b}, —COR^{a}, —CO₂R^{a} or —CONR^{a}R^{b}; or R¹ and R² together represent the residue of a carbocyclic or heterocyclic ring; one of R³, R⁴, R⁵ and R⁶ represents hydrocarbon, a heterocyclic group, halogen, cyano, trifluoromethyl, nitro, —OR^{a}, —SR^{a}, —SOR^{a}, —SO₂R^{a}, —SO₂NR^{a}R^{b}, —NR^{a}R^{b}, —NR^{a}COR^{b}, —NR^{a}CO₂R^{b}, —COR^{a}, —CO₂R^{a} or —CONR^{a} or —CONR^{a}R^{b}, and the other three of R³, R⁴, R⁵ and R⁶ independently represent hydrogen, hydrocarbon, a heterocyclic group, halogen, cyano, trifluoromethyl, nitro, —OR^{a}, —SR^{a}, —SOR^{a}, —SO₂R^{a}, —SO₂NR^{a}R^{b}, —NR^{a}R^{b}, —NR^{a}COR^{b}, —NR^{a}CO₂R^{b}, —COR^{a}, —CO₂R^{a} or —CONR^{a}R^{b} and R^{a} and R^{b} independently represent hydrogen, hydrocarbon or a heterocyclic group.

(19) Quinolone derivatives (I) in WO 93-10783 as shown below:

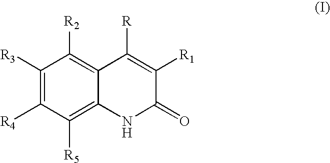

Compounds of formula I or a pharmaceutically acceptable salt thereof or a prodrug thereof wherein R represents a hydrogen atom, an amino group, a carboxy or C₂₋₆ alkoxycarbonyl group, or a group of formula -α-β-ε, in which α represents a chemical bond, an oxygen or sulphur atom, or an —NH— group; β represents a carbonyl (C=0) or sulphonyl (SO₂) group, or a straight or branched alkylene chain containing from 1 to 6 carbon atoms; and ε represents C₁₋₆alkyl, C₂₋₆ alkenyl, phenyl, —NR^{a}R^{b}, —CO₂R^{a} or —CH₂CO₂R^{a}; R¹ is a group of part formula (i) or (ii): 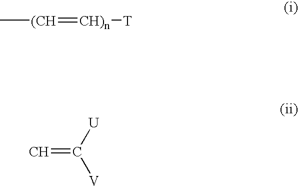
wherein U and V independently represent cyano, carboxy, —COR⁶, —CO₂R⁶, —CO.SR⁶, —CONHOH or —CONHNH₂; n is zero or 1, preferably zero; T represents cyano, carboxy, —COR⁶, —CO₂R⁶, —CO.SR⁶, —CONHOH, —CONHNH₂ or a group of formula in which the broken circle represents two non-adjacent double bonds in any position in the five-membered ring; B represents a bond or a carbonyl group (C=0); W, X, Y and Z independently represent oxygen, sulphur, nitrogen or carbon, provided that no more than one of W, X, Y and Z represents oxygen or sulphur and at least one of W, X, Y and Z is other than carbon; one of E, F and G represents nitrogen or carbon and the remainder represent carbon; A¹, A² and A³ represent one, two or three substituents not exceeding the maximum number permissible by the disposition of heteroatoms in the five- or six-membered ring, which substituents are independently selected from hydrogen, hydrocarbon, a heterocyclic group, halogen, cyano, trifluoromethyl, nitro, —OR^{a}, —SR^{a}, —SOR^{a}, —SO₂R^{a}, —SO₂NR^{a}R^{b}, —NR^{a}R^{b}, —NR^{a}COR^{b}, —NR^{a}CO₂R^{b}, —COR^{a}, —CO₂R^{a} or —CONR^{a}R^{b}; or A¹ and A² or A² and A³ together represent the residue of an aromatic or heteroaromatic ring; 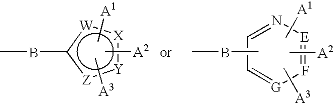
one of R², R³, R⁴ and R⁵ represents hydrocarbon, a heterocyclic group, halogen, cyano, trifluoromethyl, nitro, —OR^{a}, —SR^{a}, —SOR^{a}, —SO₂R^{a}, —SO₂NR^{a}R^{b}, —NR^{a}R^{b}, —NR^{a}COR^{b}, —NR^{a}CO₂R^{b}, —COR^{a}, —CO₂R^{a} or —CONR^{a}R^{b}, and the other three of R², R³, R⁴ and R⁵ independently represent hydrogen, hydrocarbon, a heterocyclic group, halogen, cyano, trifluoromethyl, nitro, —OR^{a}, —SR^{a}, —SOR^{a}, —SO₂R^{a}, —SO₂NR^{a}R^{b}, —NR^{a}R^{b}, —NR^{a}COR^{b}, —NR^{a}CO₂R^{b}, —COR^{a}, —CO₂R^{a} or —CONR^{a}R^{b}; or R² and R³, R³ and R⁴ or R⁴ and R⁵ together represent the residue of an aromatic or heteroaromatic ring; R⁶ represents hydrocarbon or a heterocyclic group; and R^{a} and R^{b} independently represent hydrogen, hydrocarbon or a heterocyclic group.

(20) Quinoxaline derivatives (I) in WO 93-08173 as shown below:

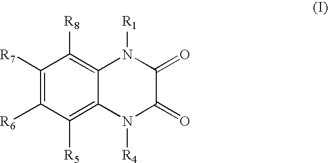

Quinoxaline derivates of the formula (I), in which R¹ is C₁₋₁₂-alkyl substituted by R², C₂₋₁₂-alkenyl substituted by R², C₂₋₁₂-alkinyl substituted by R², C₃₋₇-cycloalkyl substituted by R², —(CH₂)ₙ—C₆₋₁₂-aryl substituted by R² in the aryl or alkyl residue or —(CH₂)ₙ-hetaryl substituted by R² in the hetaryl or alkyl residue; R⁴ is hydrogen, C₁₋₁₂-alkyl substituted by R², C₂₋₁₂-alkenyl substituted by R², C₂₋₁₂-alkinyl substituted by R², (CH₂)ₙ—C₋₆₋₁₂-aryl substituted by R² in the aryl or alkyl residue, or —(CH₂)ₙ-hetaryl substituted by R² in the hetaryl or alkyl residue; R⁵, R⁶, R⁷ and R⁸ are the same or different and represent hydrogen, halogen, nitro, NR⁹R¹⁰, NHCOR¹¹, SO₂R¹², C₃₋₇-cycloalkyloxy, COR¹³, cyano, CF₃, C₁₋₆-alkyl, C₁₋₄-alkoxy or imidazole possibly substituted by cyano, C₁₋₄-alkyl or —COO—C₁₋₆-alkyl or R⁵ and R⁶ or R⁷ and R⁸ represent a condensated benzene ring, and R² stands for —CO—R³, or —PO—XY and is present once or twice in the same or a different form.

(21) Substituted 2,3-benzodiazepin-4-one (1) in WO 97-34878 as shown below:

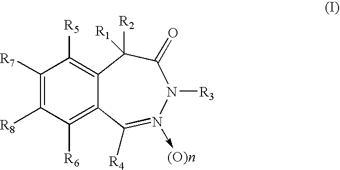

Substituted 2,3-benzodiazepin-4-one represented by formula (I) or a pharmaceutically acceptable salt or prodrug thereof, wherein: R₁ and R₂ are independently hydrogen, alkyl, haloalkyl, aryl, fused aryl, a carbocyclic group, a heterocyclic group, a heteroaryl group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, aminoalkyl or thioalkyl; or R₁ and R₂ are taken together to form a carbocycle or heterocycle; R₃ is hydrogen, alkyl, haloalkyl, aryl, fused aryl, a carbocyclic group, a heterocyclic group, a heteroaryl group, alkenyl, alkynyl, arylalkyl, arylalkenyl, arylalkynyl, heterarylalkyl, heteroarylalkenyl, heteroarylalkynyl, carbocycloalkyl, heterocycloalkyl, hydroxyalkyl, aminoalkyl, COR, CO₂R and CONRₓR_{y}, wherein R, Rₓ and R_{y} are independently hydrogen, alkyl, haloalkyl, aryl, fused aryl, carbocyclic, a heterocyclic group, a heteroaryl group, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, or aminoalkyl; or Rₓ and R_{y} are taken together to form a carbocycle or heterocycle; R₄ is substituted or unsubstituted aryl, fused aryl, a carbocyclic group, a heterocyclic group, or a heteroaryl group; R₅ and R₆ are independently hydrogen, halo, haloalkyl, aryl, fused aryl, a carbocyclic group, a heterocyclic group, a heteroaryl group, alkyl, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, nitro, amino, cyano, acylamido, hydroxy, thiol, acyloxy, azido, carboxy, carbonylamido or alkylthiol; R₇ and R₈ are independently hydrogen, halo, haloalkyl, aryl, fused aryl, a carbocyclic group, a heterocyclic group, a heteroaryl group, alkyl, alkenyl, alkynyl, arylalkyl, arylalkenyl, arylalkynyl, heteroarylalkyl, heteroarylalkenyl, heteroarylalkynyl, carbocycloalkyl, heterocycloalkyl, hydroxyalkyl, nitro, amino, cyano, acylamido, hydroxy, thiol, acyloxy, azido, alkoxy, carboxy, carbonylamido or alkylthiol; or R₇ and R₈ are taken together to form a carbocycle or heterocycle, for example, —OCH₂O, —(CH₂)₃—, —(CH₂)₄, —OCH₂CH₂O—, —CH₂N(R)CH₂—, —CH₂CH₂N(R)CH₂—, —CH₂N(R)CH₂CH₂—, —N(Me)-C(O)—O— and —N═C—C═N—, wherein R is a defined above; and n is 0 or 1.

(22) 2,3-Disubstituted-4(3H)-quinazolinone in WO97-43276 as shown below:

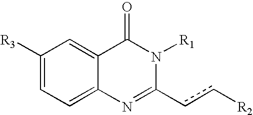

Bicyclic compounds of the formula wherein R¹ is optionally substituted phenyl of the formula Ph¹ or heteroaryl wherein said heteroaryl is selected from the group consisting of pyridin-2-yl, pyridin-3-yl and pyridin-4-yl, wherein said heteroaryl may optionally be substituted on any of the ring carbon atoms capable of forming an additional bond, up to a maximum of three substituents per ring, with a substituent selected from hydrogen, (C₁-C₆)alkyl, halogen, trifluoromethyl, amino-(CH₂)ₙ—, (C₁-C₆)alkylamino-(CH₂)ₙ—, di(C₁-C₆)alkyl-amino-(CH₂)ₙ—, (C₁-C₆)alkoxy, hydroxy(C₁-C₆)alkyl, (C₁-C₆)alkyl-O—(C₁-C₆)alkyl-, —CN, (C₁-C₆)alkyl-C—O—, (C₁-C₆)alkyl-, (C₁-C₆)alkyl-O—C—O—(C₁-C₆)alkyl, (C₁-C₆)alkyl-C—O—, hydroxy, H—C(=0)-, (C₁-C₆)allyl-C(=0)-(CH₂)ₙ—, HO—C(=0)-(CH₂)ₙ—, (C₁-C₆)alkyl-O—C(=0)-(CH₂)ₙ—, NH₂—C(=0)-(CH₂)ₙ—, (C₁-C₆)alkyl-NH—C(=0)-(CH₂)ₙ—, and di(C₁-C₆)alkyl-NH—C(=0)-(CH₂)ₙ, wherein said Ph¹ is a group of the formula 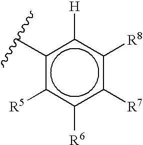
R² is phenyl of the formula Ph² or a five or six membered heterocycle, wherein said 6-membered heterocycle has the formula 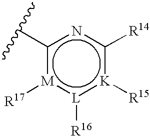
wherein “N,” is nitrogen; wherein said ring positions “K”, “L” and “M” may be independently selected from carbon or nitrogen, with the proviso that i) only one of “K, “L” or “M” can be nitrogen and ii) when “K”, “L” or “M” is nitrogen then its respective R¹⁵, R¹⁶ or R¹⁷ is absent; wherein said five membered heterocycle has the formula 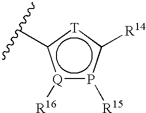
wherein said “T” is —CH—, N, NH, O or S; wherein said ring positions “P” and “Q” may be independently selected from carbon, nitrogen, oxygen or sulfur; with the proviso that only one of “P”, “Q” or “T” can be oxygen or sulfur and at least one of “P”, “Q” or “T” must be a heteroatom; wherein said Ph² is a group of the formula 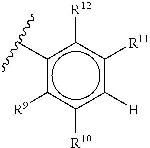
R³ is hydrogen, halo, —CN, —NO₂, CF₃, (C₁-C₆)alkyl or (C₁-C₆)alkoxy; R⁵ is hydrogen, (C₁-C₆)alkyl, halo, CF₃, (C₁-C₆)alkoxy or (C₁-C₆)alkylthiol; R⁶ is hydrogen or halo; R⁷ is hydrogen or halo; R⁸ is hydrogen or halo; R⁹ is hydrogen, halo, CF₃, (C₁-C₆)alkyl optionally substituted with one to three halogen atoms, (C₁-C₆)alkoxy optionally substituted with one to three halogen atoms, (C₁-C₆)alkylthiol, amino-CH₂)ₛ—, (C₁-C₆)alkyl-NH—(CH₂)ₛ—, di(C₁-C₆)alkyl-N—(CH₂)ₛ—, (C₃-C₇)cycloalkyl-NH—(CH₂)ₛ—, H₂N—(C=0)-(CH₂)ₛ—, (C₁-C₆)alkyl-HN—(C=0)-(CH₂)ₛ—, di(C₁-C₆)alkyl-N—(C=0)-(CH₂)ₛ—, (C₃-C₇)cycloalkyl-NH—(CH₂)ₛ—, R¹³0-(CH₂)ₛ—, R¹³0-(C=0)-(CH₂)ₛ, H(0=C)-NH—(CH₂)ₛ—, (C₁-C₆)alkyl-(0=C)-n-(CH₂)ₛ—, H(0=C)—(CH₂)ₛ—, (C₁-C₆)alkyl-(C=0)-, hydroxy, hydroxy-(C₁-C₆)alkyl, (C₁-C₆)alkyl, (C₁-C₆)alkyl-, (C₁-C₆)alkyl-O—(C₁-C₆)alkyl-, and —CN; R¹⁰ and R¹⁴ are selected, independently, from hydrogen, halo, CF₃, (C₁-C₆)alkyl optionally substituted with one to three halogen atoms, (C₁-C₆)alkoxy optionally substituted with one to three halogen atoms, (C₁-C₆)alkylthiol, amino-(CH₂)ₚ—, (C₁-C₆)alkyl-NH—(CH₂)ₚ—, di(C₁-C₆)alkyl-N—(CH₂)ₚ—, amino-(C₁-C₆)alkyl-NH—(CH₂)ₚ—, (C₁-C₆)alkyl-NH—(C₁-C₆)alkyl-NH—(CH₂)ₚ—, di(C₁-C₆)alkyl-N—(C₁-C₆)alkyl-NH—(CH₂)ₚ—, di(C₁-C₆)alkyl-N—(C₁-C₆)alkyl-N—(CH₂)ₚ—, H₂N—(C=0)-(CH₂)ₚ—, H₂N-(C=0)-(CH₂)ₚ—, (C₁-C₆)alkyl-HN—(C=0)-CH₂)ₚ—, (C₃-C₇)cycloalkyl-NY—(C=0)-(CH₂)ₚ—, R¹³0-(C=0)-(CH₂)ₚ—, H(O═C)—O—, H(O═C)—O—(C₁-C₆)alkyl-, H(0=C)—NH—(CH₂)ₚ—, (C₁-C₆)alkyl-(0=C)—NH—(CH₂)ₚ—, —CHO, H—C(C=0)-(CH₂)ₚ—, (C₁-C₆)alkyl-(C=0)-(CH₂)ₚ—, (C₁-C₆)alkyl-(0=C)—N—(CH₂)ₚ—, H(0=C)—N—(CH₂)ₚ—, HO—(C₁-C₆)Alkyl-N—(CH₂)ₚ—, (C₁-C₆)alkyl-(C=0)-O—NH-(CH₂)ₚ—, amino-(C₁-C₆)alkyl-(C=0)-O(CH₂)ₚ, (C₁-C₆)alkyl, (C₁-C₆)alkyl-NH—(C₁-C₆)alkyl-(C=0)-O—(CH₂)ₚ—, di(C₁-C₆)alkyl-N-(C₁-C₆)alkyl-(C=0)-0-(CH₂)ₚ—, amino-(C₁-C₆)alkyl-O—(C=0)-(CH₂)ₚ, (C₁-C₆)alkyl-NH—(C₁-C₆)alkyl-O—(C═O)—(CH₂)ₚ—, di(C₁-C₆)alkyl-N—(C₁-C₆)alkyl-O—(C=0)-(CH₂)p-, hydroxy, hydroxy-(C₁-C₆)alkyl-, hydroxy-(C₁-C₆)alkyl-NH—(CH₂)ₚ—, (C₁-C₆)alkyl-O—(C₁-C₆)alkyl-, —CN, piperidine-(CH₂)ₚ—, pyrrolidine-(CH₂)ₚ—, and 3-pyrroline-(CH₂)ₚ—, wherein said piperidine, pyrrolidine and 3-pyrroline of said piperidine-(CH₂)ₚ—, pyrrolidine-(CH₂)ₚ— and 3-pyrroline-(CH₂)ₚ— moieties may optionally be substituted on any of the ring carbon atoms capable of supporting and additional bond, preferably zero to two substitutents, with a substituent independently selected from halo, CF₃, (C₁-C₆)alkyl optionally substituted with one to three halogen atoms, (C₁-C₆)alkoxy optionally substituted with one to three halogen atoms, (C₁-C₆)alkylthiol, amino-(CH₂)ₚ—, (C₁-C₆)alkyl-NH—(CH₂)ₚ—, di(C₁-C₆)alkyl-N—(CH₂)ₚ—, (C₃-C₇)cycloalkyl-NH—(CH₂)ₚ—, amino-(C₁-C₆)alkyl-NH—(CH₂)ₚ—, (C₁-C₆)alkyl-NH—(C₁-C₆)alkyl-NH—(CH₂)ₚ—, di(C₁-C₆)alkyl-N—(C₁-C₆)alkyl-NH—(CH₂)ₚ—, (C₁-C₆)alkyl-O—(C₁-C₆)alkyl-, di(C₁-C₆)alkyl-N—(C₁-C₆)alkyl-N—(CH₂)ₚ—, H₂N—(C=0)-(CH₂)ₚ—, (C₁-C₆)alkyl-HN—(C=0)-(CH₂)ₚ—, di(C₁-C₆)alkyl-N—(C=0)-(CH₂)ₚ, C₃-C₇)cycloalkyl-NH—(C=0)-(CH₂)ₚ—,R³0-(CH₂)ₚ—, R¹³0-(C=0)-(CH₂)ₚ—, H(0=C)-0-, H(0=C)-0-(C₁-C₆)alkyl-, H(0=C)—NH—(CH₂)ₚ—, (C₁-C₆)alkyl-(0=C)—NH—(CH₂)ₚ—, —CHO, H—(C=0)-(CH₂)ₚ—, (C₁-C₆)alkyl-(C=0)-, (C₁-C₆)alkyl-(0=C)—N—(CH₂)ₚ—, H(0=C)-n-(CH₂)ₚ—, HO—(C₁-C₆)alkyl-N—(CH₂)ₚ—, (C₁-C₆)alkyl-(C=0)-0-NH—(CH₂)ₚ—, amino-(C₁-C₆)alkyl-(C=0)-0-(CH₂)ₚ—, (C₁-C₆)alkyl-NH—(C₁-C₆)alkyl-(C=0)-0-(CH₂)ₚ—, di(C₁-C₆)alkyl-N—(C₁-C₆)alkyl-(C=0)-0-(CH₂)ₚ—, hydroxy, hydroxy-(C₁-C₆)alkyl-, hydroxy-(C₁-C₆)alkyl-NH—(CH₂)ₚ—, and —CN; R¹¹ is hydrogen or halo; R¹² is hydrogen, —CN or halo; R¹³ is hydrogen, (C₁-C₆)alkyl, (C₁-C₆)alkyl-(C=0)-, (C₁-C₆)alkyl-O—(C=0)-, (C₁-C₆)alkyl-NH—(C=0)-, or di(C₁-C₆)alkyl-N—(C=0)-; R¹⁵ is hydrogen, —CN, (C₁-C₆)alkyl halo, CF₃, —CHO or (C₁-C₆)alkoxy; R¹⁶ is hydrogen, —CN, (C₁-C₆)alkyl, halo, CF₃, —CHO or (C₁-C₆)alkoxy; R¹⁷ is hydrogen, —CN, (C₁-C₆)alkyl, amino-(C₁-C₆)alkyl-, (C₁-C₆)alkyl-NH—(C₁-C₆)alkyl-, di(C₁-C₆)alkyl-N—(C₁-C₆)alkyl-, halo, CF₃, —CHO or (C₁-C₆)alkoxy; n is an integer from zero to 3; each p is independently an integer from zero to 3; s is an integer from zero to 4; wherein the dashed bond represented an optional double bond; with the proviso that: i) when R⁹ is hydrogen, one of R¹¹ and R¹² is other than hydrogen; ii) when R¹ is unsubstituted phenyl and R³is hydrogen then (a) R² can not be unsubstituted phenyl, thienyl or furyl or (b) R⁹ can not be CI or hydroxy when R¹⁰ and R¹¹ are hydrogen, or (c) R¹⁰ or R¹¹ can not be chloro when R⁹ and R¹² are hydrogen; iii) when R³is hydrogen; R⁶, R⁷ and R⁸ are hydrogen; and R⁵ is chloro or methyl, then (a) R² can not be unsubstituted phenyl, thienyl or furyl or (b) R¹⁰ or R¹¹ can not be chloro or (c) R⁹ or R¹² can not be hydroxy, methyl or methoxy; iv) when R³ is hydrogen or chloro; R⁵ is methyl; R⁶, R⁷ and R⁸ are hydrogen; and K, L and M equal carbon, then (a) one of R¹⁴ through R¹⁷ must be other than hydrogen or (b) R¹⁷ must be other than hydrogen or methyl; v) when R¹ is unsubstituted pyridin-2-yl and R³ is hydrogen, bromo or iodo then R² can not be unsubstituted phenyl; vi) when R⁷ is chloro; R⁵, R⁶, and R⁸ are hydrogen; and R³ is hydrogen, then (a) R² can not be unsubstituted phenyl, pyridyl, thienyl or furyl or (b) R⁹ or R¹² can not be hydroxy when R¹⁰ and R¹¹ are hydrogen; vii) when R² is unsubstituted phenyl, R⁶, R⁷ and R⁸ are hydrogen, and R³is hydrogen, then R⁵ can not be —CO₂H; viii) when R² is unsubstituted pyridin-2-yl, R⁵ and R⁷ are hydrogen, then R⁶ or R⁸ must be other than chloro; ix) when R² is unsubstituted phenyl, R³ is hydrogen, and R⁵ and R⁷ are hydrogen, then one of R⁶ or R⁸ must be other than chloro; and the pharmaceutically acceptable salts of such compounds.

(23) Fused cycloalkyl quinoxalinedione (I) in WO 98-05651 as shown below:

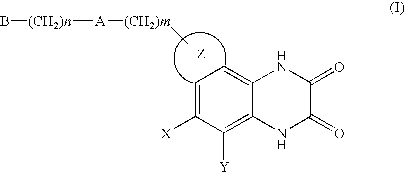

Compounds represented by the formula (I) or pharmaceutically acceptable salts thereof wherein Z is a carbocyclic fused ring having 5 to 7 carbon atoms; X and Y are independently hydrogen, halogen, nitro, cyano, —CF₃, —COOH, —CONR¹R², —COR³, —SO₂R³, imidazolyl or imidazolidinyl, wherein R¹ and R² are independently hydrogen, alkyl having 1 to 6 carbon atoms, cycloalkyl, aralkyl or join together to form a heterocyclic ring and wherein R³ is alkyl, haloalkyl, cycloalkyl, aryl or aralkyl; A is a bond, O, S, NR⁴, NR⁴CO, NR⁴CS, CONR⁴, CSNR⁴, CO or CS wherein R⁴ is hydrogen, alkyl having 1 to 6 carbon atoms, cycloalkyl, aralkyl or when n=0 then R⁴ and B may join together to form a heterocyclic ring; B is hydrogen, alkyl, alkenyl, alkynyl, aryl, aralkyl, R⁵, CN, COR⁵, PO₃R⁵₂, SO₂R⁵, or heterocyclic, wherein R⁵ is hydroxy, alkoxy, aralkoxy, aryloxy or NR¹R²; and m and n are independently 0, 1, and 2, provided that (i) m is not 0 when A is 0, CN, tetrazole or CO, except when A is CO and B is a heterocyclic or when A is 0 and B is COR⁵, PO₃R⁵₂ or SO₂R⁵; (ii) m is not 0 or 1 when A is NR⁴, except when B is COR⁵, PO₃R⁵₂ or SO₂R⁵; and (iii) n is not) when A is 0, S, NR⁴, CONR⁴ and B is NR¹R², CN, COR⁵, or PO₃R⁵₂

(24) Imidazo[1,2-a]indeno[1,2-e]pyrazine-2-carboxylic acid derivatives (I) and their salts as shown in WO 96-02544 A1 as shown below:

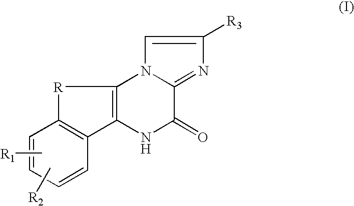

Imidazo[1,2-a]indeno[1,2-e]pyrazine-2-carboxylic acid derivatives having general formula (I) wherein R is N-alk, C(R₄)R₅, CH—R₆ or C═R₇, R₁ and R₂ are the same or different and are selected from the group consisting of hydrogen, halogen, alkyl, alkoxy, amino, —N═CH—N(alk)alk′, nitro, cyano, phenyl, imidazolyl, SO₃H, hydroxy, polyfluoralkoxy, carboxy, alkylcarbonyl, —NH—CO—NR₁₁R₁₂, —N(alk)-CO—NR₁₁R₁₂, —N(alk-Ar)—CO—NR₁₁R₁₂, —NH—CS—NR₁₁R₁₂, —N(alk)-CS—NR₁₁R₁₂, —NH—CO—NR₁₁, —NH—CS—R₂₄, —NH—C(═NR₂₇)—NR₁₀R₁₂, —N(alk)-C(═NR₂₇)—NR₁₀R₁₂. —CO—NR₁₀R₁₂, —NH—SO₂—NR₁₀R₁₂, N(alk)-SO₂—NR₁₀R₁₂, —NH—SO₂—CF₃, —NH—SO₂-alk, —NR₁₀R₁₃, S(O)ₘ-alk-Ar, —SO₂—NR₁₀R₁₂, 2-oxo-1-imidazolidinyl in which position 3 may be substituted by an alkyl group, or 2-oxo-1-perhydropyrimidinyl in which position 3 may be substituted by an alkyl group, R₃ is carboxy, alkoxycarbonyl or carboxamide, R₄ is alkyl, -alk-Het or phenylalkyl in which the phenyl group is substituted by one or more of the following: halogen, alkyl, alkoxy, nitro, amino, hydroxy, cyano, -alk-NH₂, —COOR₁₀, and -alk-COOR₁₀, R₅ is an alkyl group (the term C₁-C₁₁ alkyl represents a straight or branched alkyl chain having one to eleven carbon atoms), -alk-Het, NR₈R₉, —NH—CHO, —NH—COOR₁₇, —NH—SO₂R₂₄, —COOR₁₀, -alk-COOR₁₀, -alk-CONR₁₀R₁₈, -alk-NR₁₀R₁₈, -alk-OH, -alk-CN, phenylalkyl in which the phenyl group is substituted by one or more of the following: halogen, alkyl, alkoxy, nitro, amino, hydroxy, cyano, -alk-NH₂, —COOR₁₀, and -alk-COOR₁₀, —NH—CO—Ar in which Ar is substituted by one or more of the following: halogen, alkyl, alkoxy, nitro, amino, hydroxy, cyano, -alk-NH₂, —COOR₁₀, and -alk-COOR₁₀, —NH—CO-Het, —NH—CO-alk-Het, —NH—CO-alk-COOR₁₀, —NH—CO-alk-NR₁₀R₁₈, —NH—CO-alk-Ar in which Ar is substituted by one or more of the following: halogen, alkyl, alkoxy, nitro, amino, hydroxy, cyano, -alk-NH₂, —COOR₁₀, and -alk-COOR₁₀, pyrrol-1yl possibly substituted by —COOR₁₀, —NH—CO—NH-alk-Ar in which Ar is substituted by one or more of the following: halogen, alkyl, alkoxy, nitro, amino, hydroxy, cyano, -alk-NH₂, —COOR₁₀, and -alk-COOR₁₀, —NH—CO—NH-Het, —NH—CO—NH-alk-Het, —NH—CO—NH—Ar in which Ar is substituted by one or more of the following: halogen, alkyl, alkoxy, nitro, amino, hydroxy, cyano, -alk-NH₂, —COOR₁₀, and -alk-COOR₁₀, —NH—COalk —NH—COcycloalkyl, —NH—CO—NH-alk or —NH—CO—NH₂, or R₄ and R₅, together with the carbon atom they attached to, are a cycloalkyl group, R₆ is hydrogen, hydroxy, alkyl (the term C₁-C₁₁ alkyl represents a straight or branched alkyl chain having one to eleven carbon atoms), -alk-OH, —NR₁₄R₁₅, -alk-NR₁₄R₁₅, alk-Het, —NH—CHO, —COOalk, -alk-COOR₁₀, -alk-CO—NR₁₀R₂₁, phenylalkyl in which the phenyl group may be substituted by one or more of the following: halogen, alkyl, alkoxy, nitro, amino, hydroxy, cyano, -alk-NH₂, —COOR₁₀, and -alk-COOR₁₀, —R₁₆—COOR₁₀, —CO—COOR₁₀ or pyrrol-1yl which may be substituted by —COOR₁₀, or 2-oxo-2,5-dihydropyrrol-1-yl, R₇ is oxygen or NOH, NO-alk-COOR₁₀, NO-alk, CHR₁₉, NR₁₀, C(COOR₁₀)R₂₀ or C(CONR₁₀R₂₁)R₂₀, R₈ is hydrogen, alkyl, -alk-COOR₁₀, -alk-NR₁₀R₂₁, -alk-Het or phenylalkyl in which the phenyl group may be substituted by one or more of the following: halogen, alkyl, alkoxy, nitro, amino, hydroxy, cyano, -alk-NH₂, —COOR₁₀ and -alk-COOR₁₀, R₉ is hydrogen or alkyl, R₁₀ is hydrogen or alkyl, R₁₁ is hydrogen, alkyl (the term C₁-C₉ alkyl represents a straight or branched alkyl chain having one to nine carbon atoms), -alk-COOR₁₀, alk-Het, -alk-NR₁₂R₁₀, phenylalkyl in which the phenyl group may be substituted by one or more of the following: halogen, alkyl, alkoxy, nitro, amino, hydroxy, -alk-NH₂, carboxy, alkoxycarbonyl, cyano, and -alk-COOR₁₀ or Het, phenyl in which the phenyl group may be substituted by one or more of the following: halogen, alkyl, alkoxy, nitro, amino, hydroxy, -alk-NH₂, carboxy, alkoxycarbonyl, cyano, and -alk-COOR₁₀ or Het, R₁₂ is hydrogen or alkyl, R₁₃ is alkyl, Het or alkoxycarbonyl, R₁₄ and R₁₅ are the same or different and are each an alkyl group or R₁₄ is hydrogen and R₁₅ is hydrogen, alkyl, —COR₂₂, —CSR₂₃ or SO₂R₂₄, R₁₆ is a —CHOH or —CH(OH)-alk(C₁-C₅) chain, R₁₇ is alkyl or phenylalkyl, R₁₈ is hydrogen or alkyl, R₁₉ is hydroxy, alkyl, alk-Het, —NR₂₅R₂₆, -alk-COOR₁₀, -Het, phenyl in which the phenyl group may be substituted by one or more of the following: halogen, alkyl, alkoxy, nitro, amino, hydroxy, -alk-NH₂, —COOR₁₀, cyano, and -alk-COOR₁₀, or, phenylalkyl in which the phenyl group may be substituted by one or more of the following: halogen, alkyl, alkoxy, nitro, amino, hydroxy, -alk-NH₂, carboxy, alkoxycarbonyl, cyano, and -alk-COOR₁₀, R₂₀ is hydrogen or alkyl, R₂₁ is hydrogen or alkyl, R₂₂ is alkyl, cycloalkyl, —COOalk, -alk-COOR₁₀, phenyl in which the phenyl group may be substituted by one or more of the following: halogen, alkyl, alkoxy, nitro, amino, hydroxy, -alk-NH₂, —COOR₁₀, cyano, and -alk-COOR₁₀, phenylalkyl in which the phenyl group may be substituted by one or more of the following: halogen, alkyl, alkoxy, nitro, amino, hydroxy, -alk-NH₂, —COOR₁₀, cyano and -alk-COOR₁₀, -alk-NR₁₀R₁₂, —NH—Ar in which Ar may be substituted by one or more of the following: halogen, alkyl, alkoxy, nitro, amino, hydroxy, -alk-NH₂, —COOR₁₀, cyano, and -alk-COOR₁₀, -Het, -alk-Het, —OR₁₇, —NH-alk-Ar in which Ar may be substituted by one or more of the following: halogen, alkyl, alkoxy, nitro, amino, hydroxy, -alk-NH₂, carboxy, alkoxycarbonyl, —COOR₁₀, cyano and -alk-COOR₁₀, NH-alk-Het, —NH-alk, —NH₂ or —NH-Het, R₂₃ is —NH-alk, —NH—Ar, —NH-Het or —NH₂, R₂₄ is alkyl or phenyl, R₂₅ and R₂₆ are the same or different and are each alkyl or cycloalkyl, R₂₇ is hydrogen or alkyl.

The term alk refers to an alkyl or alkylene group. The term alk′ refers to an alkyl group, m=0,1 or 2. The term Ar refers to a phenyl group. The term Het refers to a heterocycle which is mono or poly saturated or unsaturated with four to nine carbon atoms and one or more heteroatom (O, S, N) which may be substituted with one or more of the following: alkyl, phenyl, or phenylalkyl.

Unless otherwise stated, in the above and below definitions, the alkyl or alkylene groups are a straight or branched alkyl chain having one to six carbon atom, the acyl groups have two to four carbon atoms, the cycloalkyl groups have three to six carbon atoms and the halogen are of the following: fluoride, chloride, bromide, or iodide.

Preferably, Het is one of the following rings: pyrrolyl, pyridyl, pyrimidinyl, imidazolinyl, thiazolyl, oxazolinyl, thiazolinyl, pyrazinyl, tetrazolyl, triazolyl. Each of these rings can possibly be substituted by one or more of the following: alkyl, phenyl or phenylalkyl. The preferred substitutants are methyl, phenyl or benzyl.

The compounds of the formula (I) in which R₇ is NO-alk, C(COOR₁₀)R₂₀, C(CONR₁₀R₂₁)R₂₀ or CHR₁₉ can exist as isomers (E and Z). The compounds of the present invention include the isomers E and Z and their mixtures.

The compounds of the formula (I), in which R is CH—R₆ and R₆ is —CO—COOR₁₀, can exist as tautomers (E and Z). The compounds of the present invention include the tautomers E and Z and their mixtures.

The compounds of the present invention include the eniantomers and diastereoisomers of the compounds of the formula (I), in which R is C(R₄)R₅ or CH—R₆.

(25) Phthalazine derivatives (I) in DE 196 17 862 A1 as shown below:

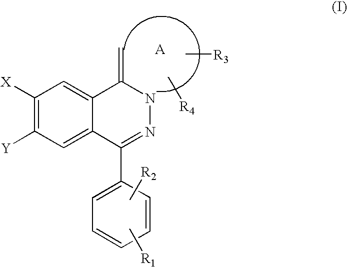

Phthalazine derivatives of the formula I wherein R¹ and R² are identical or different and hydrogen, C₁-C₆-alkyl, nitro, halogen, the group —NR⁸R⁹, —O—C₁-4-alkyl or CF₃; R³ and R⁴ are identical or different and hydrogen, an eventually substituted C₁-C₆-alkyl-, aryl- or hetaryl residue or C₃₋₇-cycloalkyl; R⁸ and R⁹ are identical or different and hydrogen, C₁-C₆-alkyl or the group —CO—C₁₋₆-alkyl, X hydrogen; Y C₁-₆-alkoxy or X and Y together —O—(CH₂)ₙ—O—; n 1, 2 or 3 mean and A forms together with nitrogen a fifemembered heterocycle, which can contain 1-3 nitrogen atoms, as well as its isomers and pharmaceutically acceptable salts thereof.

Under alkyl one has to understand a linear or branched alkyl residue as for example methyl, ethyl, propyl, isopropyl, butyl, isobutyl, sek. butyl, pentyl, isopentyl or hexyl, which can be substituted by C₁-C₆-alkoxy, halogen or C₁-C₆-alkonyl. If there is a halogenated alkyl residue present, then it can be multiple halogenated or perhalogenated such as CF₃. Under halogen one has to understand fluoride, chloride, bromide and iodide. The aryl- and hetaryl residue R³ and R⁴ can be single or multiple substituted with halogen, C₁₋₄-alkoxy or C₁₋₄-alkyl. The alyl residue can contain 6-10 carbon atoms whereby phenyl is preferred. One might mention as a hetaryl residue for example pyridinyl. With cycloalkyl one means cyclopropyl, cyclobutyl, cyclopentyl, cyclohexyl and cycloheptyl, respectively, particularly C₃₋5-cycloalkyl. Suitable as alkanoyl residues are alphatic carbonic acid residues such as formyl, acetyl, propionyl, butanoyl,caproyl, valeroyl, trimethylacetyl and others. If A together with the nitrogen atom forms a 5-membered heterocycle, then is in position 4 an exocyclic double bond. Preferred are heteroaromatics with 1-3 nitrogen atoms, whereby for example A has the following meaning:

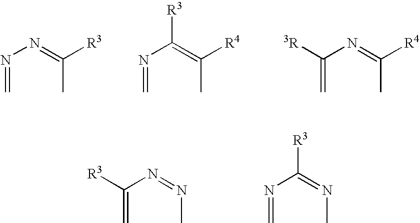

(26) 2,3-Benzodiazepine derivatives (I) in DE 196 04 920 A1 as shown below:

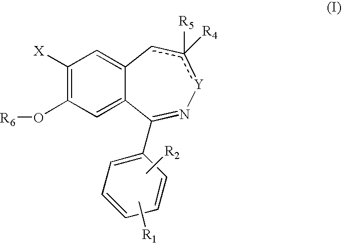

2,3-Benzodiazepine derivatives having general formula (I) wherein X is hydrogen or halogen, Y —NR³— or —N═, R¹ and R² are identical or different and hydrogen, C₁-C₆-alkyl, nitro, halogen, the group —NR⁸R⁹, —O—C₁₋₄-alkyl or —CF₃, R³ is hydrogen, the group —CO—R¹⁰, C₁₋₆-alkyl or C₃₋₇-cycloalkyl; R⁴ eventually substituted C₁-C₆-alkyl; R⁵ hydrogen or R⁴ and R⁵ together oxygen; R⁶ C₁₋₄-alkyl; R⁸ and R⁹ are identical or different and hydrogen, C₁-C₆-alkyl or —CO—C₁₋₆-alkyl; R¹⁰ hydrogen, eventually substituted C₁-C₆-alkyl, eventually substituted C₆₋₁₀-aryl, the group —NR¹¹R¹², —O—C₁₋₆-alkyl, alkyl, C₃₋₇-cycloalkyl, C₂₋₆-alkenyl or —O—C₃₋₇-cycloalkyl; R¹¹ and R¹² are identical or different and hydrogen, eventually substituted C₁-C₆-alkyl or eventually substituted C₆₋₁₀-aryl and —C . . . C . . . a double bond or single bond means as well as their isomers and pharmaceutically acceptable salts thereof.

(27) Dihydro-2,3-benzodiazepine derivatives (I) in WO 96-06606 as shown below:

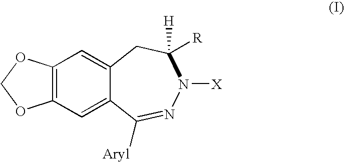

Dihydro-2,3-benzodiazepine derivatives having general formula (I) wherein R is hydrogen or C₁-C₁₀ alkyl; X is an aromatic moiety selected from phenyl, thienyl, furyl, pyridyl, imidazolyl, benzimidazolyl, benzothiazolyl and phthalazinyl which is unsubstituted or substituted with one or more moieties chosen from the group consisting of halogen, hydroxy, cyano, nitro, C₁-C₆ alkyl, C₃-C₆ cycloalkyl, C₁-C₄ alkoxy, carboxy, C₁-C₆ alkoxycarbonyl, acetyl, formyl, carboxymethyl, hydroxymethyl, amino, aminomethyl, methylenedioxy and trifluoromethyl; and “Aryl” represents p-nitrophenyl, p-aminophenyl or p-(protected amino) phenyl; or a pharmaceutically acceptable salt thereof.

(28) 3-Substituted 3H-2,3-benzodiazepine derivatives (I) in WO 96-04283 A1 as shown below: 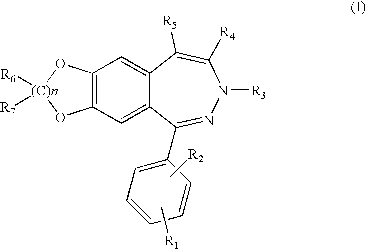
3-Substituted 3H-2,3-benzodiazepine derivatives of general formula (I) wherein R¹ and R² are identical or different and hydrogen, C₁-C₆-alkyl, nitro, halogen, the group —NR⁸R⁹, —O—C₁₋₄-alkyl or CF₃; 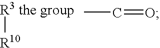
R⁴ eventually substituted C₁-C₆-alkyl; R⁵ hydrogen or eventually substituted C₁-C₆-alkyl; R⁶ and R⁷ are identical or different and hydrogen, eventually substituted C₁-C₆ alkyl or eventually substituted aryl; R⁸ and R⁹ are identical or different and hydrogen, C₁-C₆-alkyl or the group 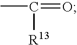
R¹⁰ hydrogen, eventually substituted C₁-C₆-alkyl, eventually substituted aryl, the group —NR¹¹R¹², —O—C₁₋₆-alkyl, C₃₋₇-cycloalkyl, C₂₋₆-alkenyl or —O—C₃₋₇-cycloalkyl; R¹¹ and R¹² are identical or different and hydrogen, eventually substituted C₁-C₆-alkyl or eventually substituted aryl; R¹³ C₁-C₆-alkyl and n stands for 1, 2 or 3; means as well as their isomers and pharmaceutically acceptable salts thereof.

(29) Heterocyclic compounds (I) in WO 95-21842 as shown in below:

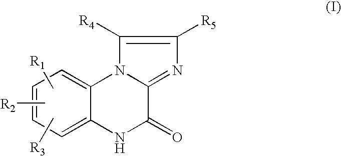

Imidazol[1,2-a]quinoxalinone derivatives of general formula (I) wherein R¹, R², R³ are the same or independently are H, alkyl, alkoxy, halogen, NO₂, NH₂, CF₃, CN, SO₂CH₃, SO₂CF₃, SO₂NR′R″ or a 5- or 6- membered N-containing heterocyclic ring, optionally substituted, and R′, R″ are independently H or alkyl; and R⁴ is H or CH₂—R⁶; and R⁶ is H, halogen, POR′″R″″, NR⁷R⁸ or a 5- or 6-membered N-containing heterocyclic ring optionally substituted, and R′″, R″″ are independently hydroxy or alkoxy; and R⁷, R⁸ are the same or independently are H, (a) or alkyl optionally substituted; and n is 1, 2, or 3; (b) CH₂OH, CHNOH, CN, (c) or (d) and R⁹ is OH, alkoxy, H or NR¹⁰R¹¹; and R¹⁰, R¹¹ are the same or independently are H, NH₂ or OH; and X is O or S; and Y is O, S or NH₂, and pharmaceutically acceptable salts thereof.

(30) 1,2,4-Triazolo[4,3-a]pyrazine-4-one derivatives and their salts in WO 95-26351 as shown below:

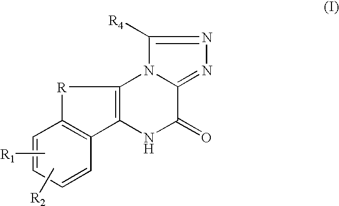

1,2,4-Triazolo[4,3-a]pyrazine-4-one derivatives having general formula (I) wherein R is N-alk, C(R₄)R₅, CH—R₆ or C═R₇. R₁ and R₂ are the same or different and are selected from the group consisting of hydrogen or halogen atoms or of alkyl, alkoxy, amino, —N═CH—N(alk)alk′, nitro, cyano, phenyl, imidazolyl, SO₃H, hydroxy, polyfluoralkoxy, carboxy, alkoxycarbonyl, —NH—CO—NR₁₁R₁₂, —N(alk)-CO—NR₁₁R₁₂, —N(alk-Ar)—CO—NR₁₁R₁₂, —NH—CS—NR₁₁R₁₂, —N(alk)-CS—NR₁₁R₁₂, —NH—CO—R₁₁, —NH—CS—R₂₄, —NH—C(═NR₂₇)—NR₁₀R₁₂, —N(alk)- C(═NR₂₇)—NR₁₀R₁₂, —Co—NR₁₀R₁₂, —NH—SO₂—NR₁₀R₁₂, N(alk)-SO₂—NR₁₀R₁₂, —NH—SO₂—CF₃, —NH—SO₂-alk, —NR₁₀R₁₃, S(O)ₘ-alk-Ar, —SO₂—NR₁₀R₁₂, 2-oxo-1-imidazolidinyl in which position 3 may be substituted by an alkyl group, or 2-oxo-1-perhydropyrimidinyl in which position 3 may be substituted by an alkyl group, R₃ is hydrogen, alkyl, cycloalkyl, alkylcycloalkyl, phenylalkyl, phenyl, Het or amino, R₄ is alkyl, -alk-Het, or phenylalkyl in which the phenyl group is substituted by one or more of the following: halogen, alkyl, alkoxy, nitro, amino, hydroxy, cyano, -alk-NH₂, —COOR₁₀, and -alk-COOR₁₀, R₅ is an alkyl group (the term C₁-C₁₀ alkyl represents a straight or branched alkyl chain having one to ten carbon atoms), -alk-Het, —NR₈R₉, —NH—CHO, —NH—COOR₁₇, —NH—SO₂—R₂₄, —COOR₁₀, -alk-COOR₁₀, -alk-CONR₁₀R₁₈, -alk-NR₁₀R₁₈, -alk-OH, -alk-CN, phenylalkyl in which the phenyl group is substituted by one or more of the following: halogen, alkyl, alkoxy, nitro, amino, hydroxy, cyano, -alk-NH₂, —COOR₁₀, and -alk-COOR₁₀, —NH—CO—Ar in which Ar is substituted by one or more of the following: halogen, alkyl, alkoxy, nitro, amino, hydroxy, cyano, -alk-NH₂, —COOR₁₀, and -alk-COOR₁₀, —NH—CO-Het, —NH—CO-alk-Het, —NH—CO-alk-COOR₁₀, —NH—CO-alk-NR₁₀R₁₈, —NH—CO-alk-Ar in which Ar is substituted by one or more of the following: halogen, alkyl, alkoxy, nitro, amino, hydroxy, cyano, -alk-NH₂, —COOR₁₀, and -alk-COOR₁₀ pyrrolyl-1 which may be substituted by —COOR₁₀, —NH—CO—NH-alk-Ar in which Ar is substituted by one or more of the following: halogen, alkyl, alkoxy, nitro, amino, hydroxy, cyano, -alk-NH₂, —COOR₁₀, and -alk-COOR₁₀, —NH—CO—NH-Het, —NH—CO—NH-alk-Het, —NH—CO—NH—Ar in which Ar may be substituted by one or more of the following: halogen, alkyl, alkoxy, nitro, amino, hydroxy, cyano, -alk-NH₂, —COOR₁₀, and -alk-COOR₁₀, —NH—COalk, —NH—COcycloalkyl, —NH—CO—NH-alk or —NH—CO—NH₂, or R₄ and R₅, together with the carbon atom they attached to, are a cycloalkyl group, R₆ is hydrogen, hydroxy, alkyl (the term C₁-C₁₀ alkyl represents a straight or branched alkyl chain having one to ten carbon atoms), -alk-OH, —NR₁₄R₁₅, -alk-NR₁₄R₁₅, -alk-Het, —NH—CHO, —COO-alk, -alk —COOR₁₀, -alk-CO—NR₁₀R₁₈, -phenylalkyl in which the phenyl group may be substituted by one or more of the following: halogen, alkyl, alkoxy, nitro, amino, hydroxy, cyano, -alk-NH₂, —COOR₁₀, and -alk-COOR₁₀, R₁₆—COOR₁₀, —CO—COOR₁₀ or pyrrolyl-1 possibly substituted by —COOR₁₀.

R₇ is oxygen, or NOH, NO-alk-COOR₁₀, NO-alk, CHR₁₉, NR₁₀, C(COOR₁₀) or C(CONR₁₀R₂₁)R₂₀, R₈ is hydrogen, alkyl, -alk-COOR₁₀, -alk-NR₁₀R₂₁, -alk-Het or phenylalkyl in which the phenyl group may be substituted by one or more of the following: halogen, alkyl, alkoxy, nitro, amino, hydroxy, cyano, -alk-NH₂, —COOR₁₀, and -alk-COOR₁₀, R₉ is hydrogen or alkyl, R₁₀ is hydrogen or alkyl, R₁₁ is hydrogen, alkyl, (the term C₁-C₉ alkyl represents a straight or branched alkyl chain having one to nine carbon atoms), alkoxy, -alk-COOR₁₀, -alk-Het, -alk-NR₁₂R₁₀, phenylalkyl in which the phenyl group may be substituted by one or more of the following: halogen, alkyl, alkoxy, nitro, amino, hydroxy, -alk-NH₂, carboxy, alkoxycarbonyl, cyano, and -alk-COOR₁₀, phenyl in which the phenyl group may be substituted by one or more of the following: halogen, alkyl, alkoxy, nitro, amino, hydroxy, -alk-NH₂, carboxy, alkoxycarbonyl, cyano and -alk-COOR₁₀ or -Het, R₁₂ is hydrogen or alkyl, R₁₃ is alkyl, Het or alkoxycarbonyl, R₁₄ and R₁₅ are the same or different and are each an alkyl moiety, or R₁₄ is hydrogen and R₁₅ is hydrogen, alkyl, —COR₂₂, —CSR₂₃ or —SO₂R₂₄, R₁₆ is a —CHOH— chain or —CH(OH)-alk(C₁-C₅), R₁₇ is alkyl or phenylalkyl, R₁₈ is hydrogen or alkyl, R₁₉ is hydroxy, alkyl, -alk-Het, —NR₂₅R₂₆, -alk-COOR₁₀, -Het, -phenyl in which the phenyl group may be substituted by one or more of the following: halogen, alkyl, alkoxy, nitro, amino, hydroxy, -alk-NH₂, —COOR₁₀, cyano, and -alk-COOR₁₀, phenylalkyl in which the phenyl group may be substituted by one or more of the following: halogen, alkyl, alkoxy, nitro, amino, hydroxy, -alk-NH₂, —COOR₁₀, cyano and -alk-COOR₁₀, R₂₀ is hydrogen or alkyl, R₂₁ is hydrogen or alkyl, R₂₂ is alkyl, cycloalkyl, —COOalk, -alk-COOR₁₀, phenyl in which the phenyl group may be substituted by one or more of the following: halogen, alkyl, alkoxy, nitro, amino, hydroxy, -alk-NH₂, —COOR₁₀, cyano and -alk-COOR₁₀, phenylalkyl in which the phenyl group may be substituted by one or more of the following: halogen, alkyl, alkoxy, nitro, amino, hydroxy, -alk-NH₂, —COOR₁₀, cyano and -alk-COOR₁₀, -alk-NR₁₀R₁₂, —NH—Ar in which Ar may be substituted by one or more of the following: halogen, alkyl, alkoxy, nitro, amino, hydroxy, -alk-NH₂, —COOR₁₀, cyano and -alk-COOR₁₀, phenylalkyl in which the phenyl group may be substituted by one or more of the following: halogen, alkyl, alkoxy, nitro, amino, hydroxy, -alk-NH₂, —COOR₁₀, cyano and -alk-COOR₁₀, -Het, -alk-Het, —OR₁₇, —NH-alk-Ar in which Ar may be substituted by one or more of the following: halogen, alkyl, alkoxy, nitro, amino, hydroxy, -alk-NH₂, —COOR₁₀, cyano and -alk-COOR₁₀, —NH-alk-Het, —NH-alk, —NH₂ or —NH-Het, R₂₃ is —NH-alk, —NH—Ar, —NH-Het or —NH₂, R₂₄ is alkyl or phenyl, R₂₅ and R₂₆ are the same or different and are each alkyl or cycloalkyl, R₂₇ is hydrogen or alkyl.

The term alk refers to an alkyl or alkylene moiety. The term alk′ refers to an alkyl moiety. m=0,1 or 2. The term Ar refers to a phenyl moiety. The term Het refers to a heterocycle which is mono- or poly-saturated or unsaturated with one to nine carbon atoms and one or more heteroatom (O, S, N) which may be substituted with one or more of the following: alkyl, phenyl, or phenylalkyl.

Unless otherwise stated, in the above and below definitions, the alkyl, alkylene or alkoxy moieties are a straight or branched chain having one to six carbon atom, the acyl moieties have two to four carbon atoms, the cycloalkyl moieties have three to six carbon atoms and the halogen atoms are selected from the following: fluoride, chloride, bromide or iodide.

Preferably, Het is one of the following rings: pyrrolyl, pyridyl, pyrimidinyl, imidazolyl, thiazolyl, oxazolinyl, thiazolinyl, pyrazinyl, tetrazolyl, triazolyl, pyrrolidinyl, piperazinyl, thienyl, furyl, azetidinyl and imidazolinyl. Each of these rings may be substituted by one or more of the following: alkyl, phenyl or phenylalkyl. The preferred substituents are methyl, phenyl or benzyl.

The preferred polyfluoroalkoxy groups are the trifluoromethoxy groups.

The compounds of the formula (I) in which R₇ is NO-alk, C(COOR₁₀)R₂₀, C(CONR₁₀R₂₁)R₂₀ or CHR₁₉ can exist as isomers (E and Z). The compounds of the present invention include the isomers E and Z and their mixtures.

The compounds of the formula (I), in which R is CH—R₆ and R₆ is —CO—COOR₁₀, can exist as tautomers (E and Z). The compounds of the present invention include the tautomers E and Z and their mixtures.

The compounds of the present invention include the eniantomers and diastereoisomers of the compounds of the formula (I), in which R is C(R₄)R₅ or CH—R₆.

(31) Imidazo(1,2-a)indeno(1,2-e)pyrazin-4-one derivatives and their salts in WO 95-26350 as shown below:

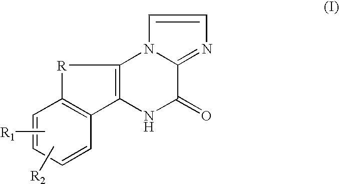

Imidazo(1,2-a)indeno(1,2-e)pyrazin-4-one derivatives having general formula (I) wherein R is C═R₃, C(R₄)R₅ or CH—R₆, R₁ and R₂ are the same or different and are selected from the group consisting of hydrogen, halogen, alkyl, alkoxy, amino, —N═CH—N(alk)alk′, nitro, cyano, phenyl, imidazolyl, SO₃H, hydroxy, polyfluoralkoxy, —COOR₇, —NH—CO—NR₈R₉, —N(alk)-CO—NR₈R₉, —N(alk-Ar)—CO—NR₈R₉, —NH—CS—NR₈R₉, —N(alk)-CS—NR₈R₉, —NH—CO—NR₁₈, —NH—CS—R₁₉, —NH—C(═NR₂₀)—NR₇R₉, —N(alk)-C(═NR₂₀)—NR₇R₉, —NH—SO₂—NR₇R₉, N(alk)-SO₂—NR₇R₉, —CO—NR₇R₉, —NH—SO₂—CF₃, —NH—SO₂-alk, —NR₉R₁₁, S(O)ₘ-alk-Ar, —SO₂—NR₇R₉, 2-oxo-1-imidazolidinyl in which position 3 may be substituted by an alkyl group, or 2-oxo-1-perhydropyrimidinyl in which position 3 may be substituted by an alkyl group, R₃ is NO-alk, CHR₁₀, NR₇, C(COOR₇)R₁₆ or C(CONR₇R₁₅)R₁₆, R₄ is alkyl, -alk-Het, -alk-Het″ or phenylalkyl in which the phenyl group is substituted by one or more of the following: halogen, alkyl, alkoxy, nitro, amino, hydroxy, cyano, -alk-NH₂, —COOR₇ and -alk-COOR₇, R₅ is —NR₁₂R₁₃, —NH—CHO, —NH—CHO, —NH—COOR₁₇, —NH—SO₂R₁₉, —COOR₇, -alk-COOR₇, -alk-CONR₇R₁₅, -alk-NR₇R₁₅, -alk-OH, -alk-CN, -alk-Het″, phenylalkyl in which the phenyl group is substituted by one or more of the following: halogen, alkyl, alkoxy, nitro, amino, hydroxy, cyano, -alk-NH₂, —COOR₇ and -alk-COOR₇, —NH—CO—Ar in which Ar is substituted by one or more of the following: halogen, alkyl, alkoxy, nitro, amino, hydroxy, cyano, -alk-NH₂, —COOR₇ and -alk-COOR₇, —NH—CO-Het, —NH—CO-Het″, —NH—CO-alk-Het, —NH—CO-alk-Het″, —NH—CO-alk-COOR₇, —NH—CO-alk-NR₇R₁₅, —NH—CO-alk-Ar in which Ar is substituted by one or more of the following: halogen, alkyl, alkoxy, nitro, amino, hydroxy, cyano, -alk-NH₂, —COOR₇, and -alk-COOR₇, —NH—CO—C(Ar)(CF₃)OCH₃, pyrrolyl-1 which may be substituted by —COOR₇, —NH—CO—NH-alk-Ar in which Ar is substituted by one or more of the following: halogen, alkyl, alkoxy, nitro, amino, hydroxy, cyano, -alk-NH₂, —COOR₇, and -alk-COOR₇, —NH—CO—NH-Het, —NH—CO—NH-Het″, —NH—CO—NH-alk-Het, —NH—CO—NH-alk-Het″, —NH—CO—NH—Ar in which Ar is substituted by one or more of the following: halogen, alkyl, alkoxy, nitro, amino, hydroxy, cyano, -alk-NH₂, —COOR₇, and -alk-COOR₇, —NH—COalk, —NH—COcycloalkyl, —NH—CO—NH-alk or —NH—CO—NH₂, R₆ is —NH—CHO, —COOalk, -alk-COOR₇, -alk-CO—NR₇R₁₅, phenylalkyl in which the phenyl group may be substituted by one or more of the following: halogen, alkyl, alkoxy, nitro, amino, hydroxy, cyano, -alk-NH₂, —COOR₇, and -alk-COOR₇, —R₁₄—COOR₇, —CO—COOR₇, —NH—COOR₁₇, —NH—CO—Ar in which Ar is substituted by one or more of the following: halogen, alkyl, alkoxy, nitro, amino, hydroxy, cyano, -alk-NH₂, —COOR₇, and -alk-COOR₇, —NH—CO-Het, —NH—CO-alk-Het, —NH—CO-Het″, —NH—CO-alk-Het″, —NH—CO-alk(C₂-C₆)—COOR₇, —NH—CO-alk(C₂-C₆)—NH₂, —NH—CO-alk-N(alk)₂, —NH—CO-alk-NHalk, —NH—CO-alk-Ar in which Ar is substituted by one or more of the following: halogen, alkyl, alkoxy, nitro, amino, hydroxy, cyano, -alk-NH₂, —COOR₇, and -alk-COOR₇, —NH—CO—C(Ar)(CF₃)OCH₃, -alk-Het″, pyrrolyl-1-may be substituted by —COOR₇, —NH—CO—NH-alk-Ar in which Ar is substituted by one or more of the following: halogen, alkyl, alkoxy, nitro, amino, hydroxy, cyano, -alk-NH₂, —COOR₇, and -alk-COOR₇, —NH—CO—NH-alk-Het, —NH—CO—NH-alk-Het″, —NH—CO—NH-Het″, or —NH—CO—NH—Ar in which Ar is substituted by one or more of the following: halogen, alkyl, alkoxy, nitro, amino, hydroxy, cyano, -alk-NH₂, —COOR₇, and -alk-COOR₇, R₇ is hydrogen or alkyl, R₈ is hydrogen, alkyl, -alk-COOR₇, -alk-Het″, -alk-Het, -alk-NR₉R₇ or phenylalkyl in which the phenyl group may be substituted by one or more of the following: halogen, alkyl, alkoxy, nitro, amino, hydroxy, -alk-NH₂, —COOR₇, cyano, -alk-COOR₇, or phenyl in which the phenyl group may be substituted by one or more of the following: halogen, alkyl, alkoxy, nitro, amino, hydroxy, -alk-NH₂, —COOR₇, cyano, -alk-COOR₇, -Het or -Het″, R₉ is hydrogen or alkyl, R₁₀ is -alk-COOR₇, -Het″, -alk-Het″, phenyl in which the phenyl group may be substituted by one or more of the following: halogen, alkyl, alkoxy, nitro, amino, hydroxy, -alk-NH₂, COOR₇, cyano, -alk —COOR₇, or phenylalkyl in which the phenyl group may be substituted by one or more of the following: halogen, alkyl, alkoxy, nitro, amino, hydroxy, -alk-NH₂, -COOR₇, cyano, -alk-COOR₇, R₁₁ is alkyl, -Het, -Het″ or alkylcarbonyl, R₁₂ is hydrogen, alkyl, -alk-COOR₇, -alk-NR₇R₁₅, -alk-Het, -alk-Het″, or phenylalkyl in which the phenyl group may be substituted by one or more of the following: halogen, alkyl, alkoxy, nitro, amino, hydroxy, -alk-NH₂, carboxy, alkoxycarbonyl, cyano, and -alk-COOR₇, R₁₃ is hydrogen or alkyl, R₁₄ is a —CHOH— or —CHOH-alk(C₁-C₅) chain, R₁₅ is hydrogen or alkyl, R₁₆ is hydrogen or alkyl, R₁₇ is alkyl or phenylalkyl, R₁₈ is hydrogen, or an alkyl moiety (the term C₁-C₉ alkyl represents a straight or branched alkyl chain having one to nine carbon atoms), alkoxy, -alk-COOR₇, -alk-Het″, -alk-Het, , -alk-NR₉R₇, phenylalkyl in which the phenyl group may be substituted by one or more of the following: halogen, alkyl, alkoxy, nitro, amino, hydroxy, -alk-NH₂, —COOR₇, cyano and -alk-COOR₇, phenyl in which the phenyl group may be substituted by one or more of the following: halogen, alkyl, alkoxy, nitro, amino, hydroxy, -alk-NH₂, cyano, -alk-COOR₇, Het or Het″, R₁₉ is alkyl or phenyl, R₂₀ is hydrogen or alkyl.

The term alk refers to an alkyl or alkylene moiety. The term alk′ refers to an alkyl moiety. The term Ar refers to a phenyl moiety. m=0, I or 2. The term Het refers to a heterocycle which is mono- or poly-saturated or unsaturated with four to nine carbon atoms and one or more heteroatom (O, S, N). The term Het″ refers to a heterocycle which is mono- or poly-saturated or unsaturated with one to three carbon atoms and one or more heteroatom (O, S, N) may be substituted with one or—or poly-saturated or insaturated with four to nine carbon atoms and one or more heteroatom (O, S, N) may be substituted with one or more of the following: alkyl, phenyl, or phenylalkyl. Provided that when R₁ and R₂ are hydrogen, R is CHR₆, R₆ is alk-Het″ in which alk is alkyl (C₁) and Het″ is not 2-imidazol.

Unless otherwise stated, in the above and below definitions, the alkyl or alkylene moieties are a straight or branched chain having one to six carbon atom, the cycloalkyl moieties have three to six carbon atoms and the halogen atoms are selected from the following: fluoride, chloride, bromide, or iodide.

Preferably, Het is one of the following cycles: pyrrolyl, pyridyl, pyrimidinyl, morpholinyl, pyrazinyl, pyrrolidinyl, piperazinyl, piperidinyl, thienyl and furyl. Het″ is one of the following: pyrrolyl, pyridyl, pyrimidinyl, imidazolyl, thiazolyl, thiazolinyl, pyrazinyl, tetrazolyl, triazolyl, oxazolyl, pyrrolidinyl, azetidinyl, piperazinyl, piperidinyl, thienyl, oxazolinyl, furyl and imidazolinyl. Each of these rings may be substituted by one or more of the following: alkyl, phenyl or phenylalkyl. The preferred substitutants are methyl, phenyl or benzyl.

The prefered polyfluoroalkoxy groups are the trifluoromethoxy groups. The compounds of the formula (I) in which R₃ is NO-alk, C(COOR₇)R₁₆, C(CONR₇R₁₅)R₁₆ or CHR₁₀ can exist as isomers (E and Z). The compounds of the present invention include the isomers E and Z and their mixtures.

The compounds of the formula (I), in which R is CH—R₆ and R₆ is —CO—COOR₇, can exist as tautomers (E and Z). The compounds of the present invention include the tautomers E and Z and their mixtures.

The compounds of the present invention include the eniantomers and diastereoisomers of the compounds of the formula (I), in which R is C(R₄)R₅ or CH—R₆.

The compounds of of the present invention include compounds of the formula (1) in which R, R₁ and R₂ are as defined previously except for when: a) R₁ and R₂ are hydrogen, R is CHR₆, R₆ is -alk-Het″ in which alk is an alkyl moiety (C₁) and Het″ is a 2-imidazolyl moiety, b) R₁ and R₂ are hydrogen, R is CHR₆, R₆ is —NHCHO or alk-COOR₇ in which R₇ is hydrogen or a terbutyl group, c) R₁ and R₂ are hydrogen, R is C═R₃, R₃ is CHR₁₀ and R₁₀ is a 2-imidazolyl moiety, d) R₁ is hydrogen, R₂ is CH₆ and R₆ is —NHCHO. The preferred compounds are those with R₁ in position -7 or -8.

(32) Indeno[1,2-e]pyrazine-4-one (I) in WO 95-26349 as shown below:

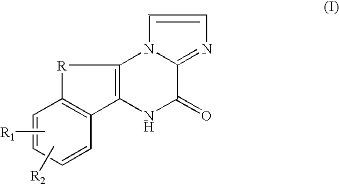

Indeno[1,2-e]pyrazine-4-one of formula (I), wherein R is a substituted nitrogen, oxygen or sulphur atom or a radical C═R₃, C(R₄)R₅ or CH—R₆; R₁ is a hydroxy radical, polyfluoroalkoxy, carboxy, alkoxycarbonyl, —NH—CHO or —NH—CO—N(alk)Ar where Ar is optionally substituted, —N(alk)-CO—NR₈R₉, —N(alk-Ar)—CO—NR₈R₉, —NH—CO—NR₉R₁₂, —NH—CS—NR₈R₉, —N(alk)-CS—NR₈—R₉, —NH—CO—R₁₀,—NH—CS—R₂₀,—NH—C(═NR₂₁)—NR₇R₉,—N(alk)-C(═NR₂₁)—NR₇R₉,—NH—SO₂—NR₇R₉, N(alk)-SO₂—NR⁷R⁹, —CO—NR₇R₉, —NH—SO₂—CF₀, —NH—SO₂-alk, —NR₉R₁₁, —S(O)ₘ-alk-Ar, —SO₂—NR₇R₉, optionally 3-substituted 2-oxo-1 imidazolidinyl or optionally 3-substituted 2-oxo-1 perhydropyrimidinyl; R₂ is a hydrogen or halogen atom or an akyl radical, alkoxy, amino, —NH—CO—NH—Ar, N═CH.N(alk)alk′, nitro, cyano, phenyl, imidazolyl, acylamino, SO₃H, hydroxy, polyfluoroalkoxy, carboxy, alkoxycarbonyl, —NH—CHO, —NH—CO—N(alk)Ar where Ar is optionally substituted, —N(alk)-CO—NR₈R₉, —N(alk-Ar)—CO—NR₈R₉, —NH—CO—NR₉R₁₂ —NH—CS—NR₈R₉, —N(alk)-CS—NR₈R₉, —NH—CO—R₁₀, —NH—CS—R₂₀, —NH—C(═NR₂₁)—NR₇R₉,—N(alk)-C(═NR₂₁)—NR₇R₉, —NH—SO₂—NR₇R₉, —N(alk)-SO₂—NR₇R₉, —CO—NR₇R₉, —NH—SO₂—CF₃, —NH—SO₂-alk, —NR₉R₁₁, —S(O)ₘ-alk-Ar, —SO₂—NR₇R₉, optionally 3-substituted 2-oxo-1-imidazolidinyl or optionally 3-substituted 2-oxo-1-perhydropyrimidinyl; R₃ is an oxygen atom or a NOH, NO-alk-COOX or CH—R₁₃ radical, R⁴ is an alkyl radical; -alk-Het or -alk-Ar; R₅ is a straight or branched C₁₋₁₁ alkyl radical, -alk-Het or -alk-Ar, or R₄ and R₅, taken together with the carbon atom to which they are attached, form a cycloalkyl radical; R₆ is a hydrogen atom radical or a hydroxy radical, straight or branched C₁-₁₁ alkyl, —NR₁₄R₁₅, -alk-OH, -alk-NR₁₄R₁₅, -alk-Ar or -alk-Het; and salts thereof

(33) Imidazo[1,2-a]pyrazine-4-one derivatives (I) in WO95-26352 as shown below:

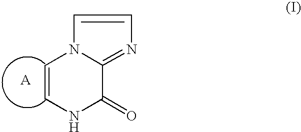

Compounds of formula (I), wherein ring A is selected from rings 1, 2 and 3, wherein R is a CH₂ radical or a sulphur, oxygen or nitrogen atom substituted by an alkyl radical, and salts thereof.

(34) 5H-Indeno[1,2-b]pyrazine-2,3-dione derivatives and their salts (I) in WO 95-26342 as shown below:

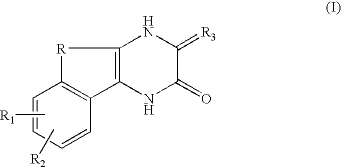

5H-Indeno[1,2-b]pyrazine-2,3-dione of formula (I), wherein R is N-alk, C(R₄)R₅, CH—R₆ or C═R₇, R₁ and R₂ are the same or different and are selected from the group consisting of hydrogen, halogen, alkyl, alkoxy, amino, —N═CH—N(alk)alk′, nitro, cyano, phenyl, imidazolyl, SO₃H, hydroxy, polyfluoralkoxy, carboxy, alkylcarbonyl, —NH—CO—NR₁₁R₁₂, —N(alk)-CO—NR₁₁R₁₂, —N(alk-Ar)—CO—NR₁₁R₁₂, —NH—CS—NR₁₁R₁₂, —N(alk)-CS—NR₁₁R₁₂, —NH—CO—NR₁₁, —NH—CS—R₂₄, —NH—C(═NR₂₇)—NR₁₀R₁₂, —N(alk) C(═NR₂₇)—NR₁₀R₁₂, —CO—NR₁₀R₁₂, —NH—SO₂—NR₁₀R₁₂, N(alk)-SO₂—NR₁₀R₁₂, —NH—SO₂—CF₃, —NH—SO₂-alk, —NR₁₀R₁₃, S(O)ₘ-alk-Ar, —SO₂—NR₁₀R₁₂, 2-oxo-1-imidazolidinyl in which position 3 may be substituted by an alkyl group, or 2-oxo-1-perhydropyrimidinyl in which position 3 may be substituted by an alkyl group, R₃ is oxygen, NOH, NOalk or NOalkAr, R₄ is alkyl, -alk-Het or phenylalkyl in which the phenyl group is substituted by one or more of the following: halogen, alkyl, alkoxy, nitro, amino, hydroxy, cyano, -alk-NH₂, —COOR₁₀, and -alk-COOR₁₀, R₅ is an alkyl group (the term C₁-C₁₁ alkyl represents a straight or branched alkyl chain having one to eleven carbon atoms), -alk-Het, NR₈R₉, —NH—CHO, —NH—COOR₁₇, —NH—SO₂R₂₄, —COOR₁₀, -alk-COOR₁₀, -alk-CONR₁₀R₁₈, -alk-NR₁₀R₁₈, -alk-OH, -alk-CN, phenylalkyl in which the phenyl group is substituted by one or more of the following: halogen, alkyl, alkoxy, nitro, amino, hydroxy, cyano, -alk-NH₂, —COOR₁₀, and -alk-COOR₁₀, —NH—CO—Ar in which Ar is substituted by one or more of the following: halogen, alkyl, alkoxy, nitro, amino, hydroxy, cyano, -alk-NH₂, —COOR₁₀, and -alk-COOR₁₀, —NH—CO-Het, —NH—CO-alk-Het, —NH—CO-alk-COOR₁₀, —NH—CO-alk-NR₁₀R₁₈, —NH—CO-alk-Ar in which Ar is substituted by one or more of the following: halogen, alkyl, alkoxy, nitro, amino, hydroxy, cyano, -alk-NH₂, —COOR₁₀, and -alk-COOR₁₀, pyrrolyl-1wich may be substituted by —COOR₁₀, —NH—CO—NH-alk-Ar in which Ar is substituted by one or more of the following: halogen, alkyl, alkoxy, nitro, amino, hydroxy, cyano, -alk-NH₂, —COOR₁₀, and -alk-COOR₁₀, —NH—CO—NH-Het, —NH—CO—NH-alk-Het, —NH—CO—NH—Ar in which Ar is substituted by one or more of the following: halogen, alkyl, alkoxy, nitro, amino, hydroxy, cyano, -alk-NH₂, —COOR₁₀, and -alk-COOR₁₀, —NH—COalk, —NH—COcycloalkyl, —NH—CO—NH-alk or —NH—CO—NH₂, or R₄ and R₅, together with the carbon atom they attached to, are a cycloalkyl group, R₆ is hydrogen, hydroxy, alkyl (the term C₁-C₁₁ alkyl represents a straight or branched alkyl chain having one to eleven carbon atoms), -alk-OH, —NR₁₄R₁₅, -alk-NR₁₄R₁₅, alk-Het, —NH—CHO, —COOalk, -alk-COOR₁₀, -alk-CO—NR₁₀R₁₈, phenylalkyl in which the phenyl group may be substituted by one or more of the following: halogen, alkyl, alkoxy, nitro, amino, hydroxy, cyano, -alk-NH₂, —COOR₁₀, and -alk-COOR₁₀, —R₁₆—COOR₁₀, —CO—COOR₁₀ or pyrrolyl-1 may be substituted by —COOR₁₀, R₇ is oxygen or NOH, NO-alk-COOR₁₀, NO-alk, CHR₁₉, C(COOR₁₀)R₂₀ or C(CONR₁₀R₂₁)R₂₀, R₈ is hydrogen, alkyl, -alk-COOR₁₀, -alk-NR₁₀R₂₁, -alk-Het or phenylalkyl in which the phenyl group may be substituted by one or more of the following: halogen, alkyl, alkoxy, nitro, amino, hydroxy, cyano, -alk-NH₂, —COOR₁₀ and -alk-COOR₁₀, R₉ is hydrogen or alkyl, R₁₀ is hydrogen or alkyl, R₁₁ is hydrogen, alkyl (the term C₁-C₉ alkyl represents a straight or branched alkyl chain having one to nine carbon atoms), alkoxy, -alk-COOR₁₀, alk-Het, -alk-NR₁₂R₁₀, phenylalkyl in which the phenyl group may be substituted by one or more of the following: halogen, alkyl, alkoxy, nitro, amino, hydroxy, -alk-NH₂, carboxy, alkoxycarbonyl, cyano and -alk-COOR₁₀, phenyl in which the phenyl group may be substituted by one or more of the following: halogen, alkyl, alkoxy, nitro, amino, hydroxy, -alk-NH₂, carboxy, alkoxycarbonyl, cyano and -alk-COOR₁₀ or -Het, R₁₂ is hydrogen or alkyl, R₁₃ is alkyl, Het or alkoxycarbonyl, R₁₄ and R₁₅ are the same or different and are each an alkyl group or R₁₄ is hydrogen and R₁₅ is hydrogen, alkyl, —COR₂₂, —CSR₂₃ or SO₂R₂₄, R₁₆ is a —CHOH or —CH(OH)alk(C₁-C₅) chain, R₁₇ is alkyl or phenylalkyl, R₁₈ is hydrogen or alkyl, R₁₉ is hydroxy, alkyl, alk-Het, —NR₂₅R₂₆, -alk-COOR₁₀, -Het, phenyl in which the phenyl group may be substituted by one or more of the following: halogen, alkyl, alkoxy, nitro, amino, hydroxy, -alk-NH₂, —COOR₁₀, cyano, and -alk-COOR₁₀ or phenylalkyl in which the phenyl group may be substituted by one or more of the following: halogen, alkyl, alkoxy, nitro, amino, hydroxy, -alk-NH₂, —COOR₁₀, cyano, and -alk-COOR₁₀, R₂₀ is hydrogen or alkyl, R₂₁ is hydrogen or alkyl, R₂₂ is alkyl, cycloalkyl, —COOalk, -alk-COOR₁₀, phenyl in which the phenyl group may be substituted by one or more of the following: halogen, alkyl, alkoxy, nitro, amino, hydroxy, -alk-NH₂, —COOR₁₀, cyano, and -alk-COOR₁₀, phenylalkyl in which the phenyl group may be substituted by one or more of the following: halogen, alkyl, alkoxy, nitro, amino, hydroxy, -alk-NH₂, —COOR₁₀, cyano, and -alk-COOR₁₀, -alk-NR₁₀R₁₂, —NH—Ar in which Ar may be substituted by one or more of the following: halogen, alkyl, alkoxy, nitro, amino, hydroxy, -alk-NH₂, —COOR₁₀, cyano, and -alk-COOR₁₀, -phenylalkyl in which the phenyl group may be substituted by one or more of the following: halogen, alkyl, alkoxy, nitro, amino, hydroxy, -alk-NH₂, —COOR₁₀, cyano, and -alk-COOR₁₀, -Het, -alk-Het, —OR₁₇, —NH-alk-Ar in which Ar may be substituted by one or more of the following: halogen, alkyl, alkoxy, nitro, amino, hydroxy, -alk-NH₂, —COOR₁₀, cyano and -alk-COOR₁₀, NH-alk-Het, —NH-alk, —NH₂ or —NH-Het, R₂₃ is —NH-alk, —NH—Ar, —NH-Het or —NH₂, R₂₄ is alkyl or phenyl, R₂₅ and R₂₆ are the same or different and are each alkyl or cycloalkyl, R₂₇ is hydrogen or alkyl.

The term alk refers to an alkyl or alkylene group. The term alk′ refers to an alkyl group. m=0,1 or 2. The term Ar refers to a phenyl group. The term Het refers to a heterocycle which is mono or poly saturated or unsaturated with four to nine carbon atoms and one or more heteroatom (O, S, N) may be substituted with one or more of the following: alkyl, phenyl, or phenylalkyl. Provided that when R₁ and R₂ are hydrogen and R₃ is oxygen, R is not (a) C═R₇ in which R₇ is oxygen or NOH, (b) CH—R₆ in which R is hydroxy.

Unless otherwise stated, in the above and below definitions, the alkyl or alkylene groups are a straight or branched alkyl chain having one to six carbon atom, the acyl groups have two to four carbon atoms, the cycloalkyl groups have three to six carbon atoms and the halogen are of the following: fluoride, chloride, bromide, or iodide.

Preferably, Het is one of the following rings: pyrrolyl, pyridyl, pyrimidinyl, thiazolyl, oxazolinyl, thiazolinyl, pyrazinyl, tetrazolyl, triazolyl, pyrrolidinyl, piperazinyl, piperidinyl, thienyl, furyl, azetidinyl, imidazolinyl. Each of these rings may be substituted by one or more of the following: alkyl, phenyl or phenylalkyl. The preferred substituents are methyl, phenyl or benzyl.

The preferred polyfluoroalkoxy groups are the trifluoromethoxy groups. The compounds of the formula (I) in which R is C═R₇ , with R₇ being NO-alk, C(COOR₁₀)R₂₀, C(CONR₁₀R₂₁)R₂₀ or CHR₁₉ and/or with R₃ being NOH, NOalk or NOalkAr, can exist as isomers (E and Z). The compounds of the present invention include the isomers E and Z and their mixtures.

The compounds of the formula (I), in which R is CH—R₆ and RF is —CO—COOR₁₀, can exist as tautomers (E and Z). The compounds of the present invention include the tautomers E and Z and their mixtures. The compounds of the present invention include the enantiomers and diastereoisomers of the compounds of the formula (I), in which R is C(R₄)R₅ or CH—R₆.

(35) Quinazoline-2,4-dione (I) in WO 95-19346 as shown below:

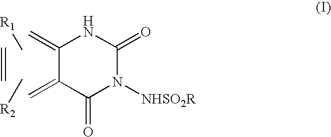

Compounds of formula I wherein R is (C₁₋₆) alkyl or phenyl optionally mono-, di- or trisubstituted by halogen, (C₁₋₄)alkyl, (C₁₋₄)alkoxy, nitro, trifluoromethyl, amino, (C₁₋₄)alkylamino, di(C₁₋₄)alkylamino, cyano, (C₁₋₄)alkylsulfonyl, phenylsulfonyl or sulfonylamino, R₁ and R₂ independently are hydrogen, hydroxy, (C₁₋₄)alkyl, (C₁₋₄)alkoxy, (C₂₋₅)alkenyl, halogen, trifluoromethyl, nitro, amino, (C₁₋₄)alkylamino, benzyloxy, benzoylamino, carboxy, cyano, (C₁₋₄)alkoxy-carbonyl, (C₁₋₄)alkylsulfonyl, phenylsulfonyl, sulfonylamino, (C₂₋₅)alkanoylamino or phenyl optionally substituted by (C₁₋₄)alkyl, halogen or nitro, provided that R₁ and R₂ are not both hydrogen if R is unsubstituted phenyl, or R₁ and R₂ on adjacent carbon atoms together form a group —CH═CH—CH═CH—, or a salt thereof. Alkyl and alkoxy groups and moieties in the compounds of formula I may be straight- or branched-chained. Halogen means fluorine, chlorine, bromine or iodine. The compounds of formula I may form cationic salts, e.g. alkali metal or ammonium salts deriving from the sulfonamido group or when a carboxyl group is present. Depending on the nature of the substituents defined above, the compounds of formula I may also form acid addition salts. The tautomeric forms of the compounds of formula I are also embraced.

(36) 3,4-Dihydro-2H-1,2,4-benzothiadiazine 1,1-dioxide-3-carboxylic acid derivatives (I) in WO 95-07899 as shown in below:

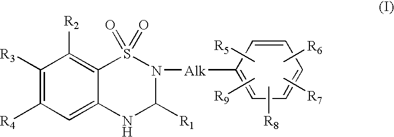

Compounds of formula (I) wherein R₁ is a carboxy, alkoxycarbonyl, tetrazolyl, —CO—NH₂, —CO—NH-alk, —CO—N(alk)₂ —CO—NHOH, —CO—N(alk)OH, —CO—NH—O—R₁₀, —CO—N(alk)-OR₁₀ radical or a group convertible in vivo into a carboxy radical, R₂, R₃ and R₄, which are the same or different, are hydrogen or halogen atoms or alkyl or alkoxy radicals, R₅ is a hydroxy, —NHOH, —NH—CO—NH₂, —CH₂—NH₂, hydroxyalkyl, alkoxyalkyl, or -alk=NOH radical, R6, R7, R8 and R9, which are the same or different, are hydrogen or halogen atoms or alkyl, alkoxy, polyfluoroalkyl, amino, nitro, cyano, vinyl, polyfluoroalkoxy, alkoxycarbonyl, carboxy, phenylalkyloxy, phenylalkyl, benzoylamino,phenylcarbonyl, hydroxy, —NHOH, —NH—CO—NH₂, —CH2- NH2, hydroxyalkyl, alkoxyalkyl, -alk=NOH or phenoxy, with the phenyl ring being optionally substituted by one or several substituents selected from the halogen atoms and the alkyl, alkoxy or polyfluoroalkyl radicals, R₁₀ is an alkyl or phenylalkyl radical and alk is an alkyl or alkylene radical. The invention also concerns the salts of thereof, the preparation thereof, and drugs containing same.

(37) Imidazo(1,2-a)pyrazin-4-one derivatives (I) in WO95-02602 as shown below.

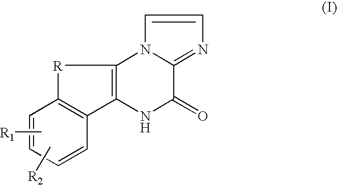

Compounds of formula (I), wherein R is an oxygen or sulphur atom or an NH or N-alk radical, and each of R₁ and R₂, which are the same or different , is a hydrogen or halogen atom or an alkyl, alkoxy, amino, acylamino, —NH—CO—NH—Ar,—N=CH—N(alk)alk', nitro, cyano, phenyl, imidazolyl or SO₃H radical, the preparation thereof, and drugs containing such compounds.

(38) 2,3-Benzodiazepine derivatives (I) and (II) in GB 2 311 779 A as shown below.

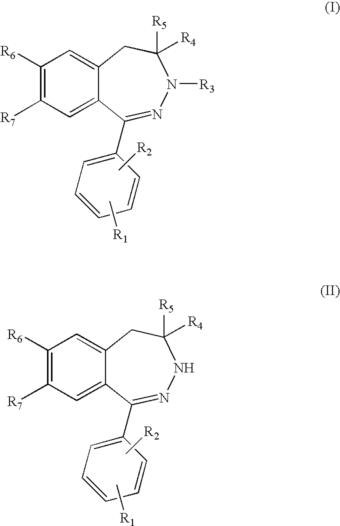

Non-competitive AMPA antagonistic compounds of the formula I, wherein R¹ and R² represent, independently, a hydrogen, a halo, a C₁₋₄ alkyl group, a C₁₋₄ alkoxy group, a nitro group, a trifluoromethyl group or a group of the formula —NR⁸R⁹, wherein R⁸ and R⁹ stand, independently, for a hydrogen, a C₁₋₄ alkyl group or a group of the formula —COR¹⁰, wherein R¹⁰ is a hydrogen, a C₁₋₆ alkyl group that can be substituted, a C₆₋₁₀ aryl group, a C₁₋₄ alkoxy group, a C₃₋₅ cycloalkyl group, a C₂₋₆ alkenyl group a C₃₋₅ cycloalkoxy group or a group of the formula —NR¹¹R¹², wherein R¹¹ and R¹² mean, independently, a hydrogen, a C₁₋₄ alkyl group, a C₃₋₅ cycloalkyl group or a C₆₋₁₀ aryl group, R³ represents a C₁₋₄ alkyl groups a C₃₋₅ cycloalkyl group or a group of the formula —CO—R¹³, wherein R¹³ has the same definitions given in relation to R¹⁰, R⁴ and R⁵ mean, independently, a hydrogen or a C₁₋₃ allyl group, R⁶ and R⁷ are, independently, a hydrogen, a chloro or a bromo, with the provision that if one of R⁶ and R⁷ stands for a hydrogen, the other is different from hydrogen, as well as the isomers thereof and the acid addition salts of the compounds or the isomers.

(39) Tetramic acid derivatives (I) in GB 2 266 888 A as shown below:

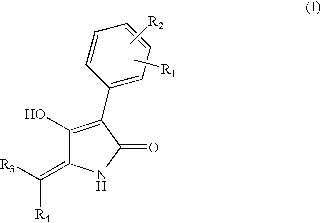

Wherein R¹ and R² independently represent hydrogen, hydrocarbon, a heterocyclic group, halogen, cyano, trifluoromethyl, nitro, —OR^{a}, —SR^{a}, —SOR^{a}, —SO₂R^{a}, —SO₂NR^{a}R^{b}, —NR^{a}R^{b}, —NR^{a}COR^{b}, —NR^{a}CO₂R^{b}, —COR^{a}, —CO₂R^{a} or —CONR^{a}R^{b}; or R¹ and R² together represent the residue of a carbocyclic or heterocyclic ring; R³ and R⁴ independently represent hydrogen, hydrocarbon, a heterocyclic group, trifluoromethyl, —OR^{c}, —SR^{c}, —SOR^{a}, —SO₂R^{a}, —SO₂NR^{a}R^{b}, —COR^{a}, —CO₂R^{a} or —CONR^{a}R^{b}, provided that R³ does not represent C₂₋₅ alkoxycarbonyl when R⁴ represents an optionally substituted phenyl group; R^{a} and R^{b} independently represent hydrogen, hydrocarbon or a heterocyclic group; and R^{c} represents hydrocarbon or a heterocyclic group.

(40) Pyrrolo-pyridazinone derivatives (I) in GB 2 265 372 A as shown below:

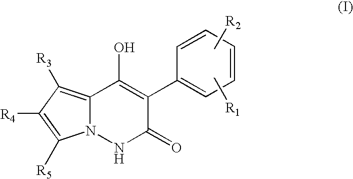

Pyrrolo-pyridazinone derivatives, wherein R¹ and R² independently represent hydrogen, hydrocarbon, a heterocyclic group, halogen, cyano, trifluoromethyl, nitro, —OR^{a}, —SR^{a}, —SOR^{a}, —SO₂R^{a}, —SO₂NR^{a}R^{b}, —NR^{a}R^{b}, —NR^{a}COR^{b}, —NR^{a}CO₂R^{b}, —COR^{a}, —CO₂R^{a} or —CONR^{a}R^{b}; or R¹ and R² together represent the residue of a carbocyclic or heterocyclic ring; R³, R⁴ and R⁵ independently represent hydrogen, hydrocarbon, a heterocyclic group, halogen, cyano, trifluoromethyl, nitro, —OR^{a}, —SR^{a}, —SOR^{a}, —SO₂R^{a}, —SO₂NR^{a}R^{b}, —NR^{a}R^{b}, —NR^{a}COR^{b}, —NR^{a}CO₂R^{b}, —COR¹, —CO₂R^{a} or CONR^{a}R^{b}; and R^{a} and R^{b} independently represent hydrogen, hydrocarbon or a heterocyclic group.

(41) 2-Phenylpyridazino[4,5-b]indole-1,4-dione derivatives (I) in GB 2 290 292 A as shown below:

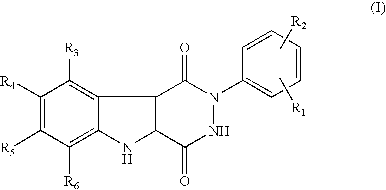

Compound of formula I, or a salt or prodrug thereof: wherein R¹ and R² independently represent hydrogen, hydrocarbon, a heterocyclic group, halogen, cyano, trifluoromethyl, nitro, —OR^{a}, —SR^{a}, —SOR^{a}, —SO₂R^{a}, —SO₂NR^{a}R^{b}, —NR^{a}R^{b}, —NR^{a}COR^{b}, —NR^{a}CO₂R^{b}, —COR^{a}, —CO₂R^{a} or —CONR^{a}R^{b}; or R¹ and R² together represent the residue of a carbocyclic or heterocyclic ring; R³, R⁴, R⁵ and R⁶ independently represent hydrogen, hydrocarbon, a heterocyclic group, halogen, cyano, trifluoromethyl, nitro, —OR^{a}, —SR^{a}, —SOR^{a}, —SO₂R^{a}, —SO₂NR^{a}R^{b}, —NR^{a}R^{b}, —NR^{a}COR^{b}, —NR^{a}CO₂R^{b},—COR^{a}, —CO₂R^{a} or —CONR^{a}R^{b}; and R^{a} and R^{b} independently represent hydrogen, hydrocarbon or a heterocyclic group.

(42) Arylthioxaline derivatives (I) in Tokkaihei 8-59660 as shown below:

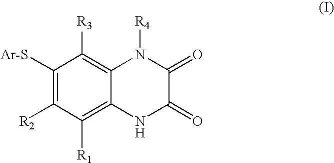

Arylthioxaline derivatives of the formula (I) and its related salts, wherein R1 is hydrogen, halogen, or nitro, R2 is hydrogen, halogen, nitro, cyano, or trihalogenomethyl, R3 is hydrogen, halogen, or nitro, R4 is hydrogen, optionally substituted lower alkyl, or optionally substituted lower cycloalkyl, and Ar is optionally substituted aromatic heterocyclic ring having at least one nitrogen atom.

(43) Hydroxyquinoxalinedione derivatives in Tokkaihei 7-165756 as shown below:

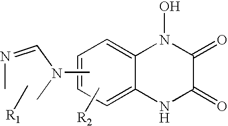

The present invention relates to hydroxyquinoxalinedione derivatives of the above formula and its related salt, wherein R1 is hydrogen or lower alkyl, and R2 is nitro or trifluoromethyl.

(44) Imidazo[1,2-a]pyrazin-4-one (I) in WO 95-02601 as shown below:

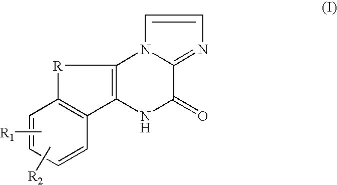

Compounds of formula (I), wherein either R is C═R₃, C(R₄) R₅ or CH—R₆, R₁ and R₂ are hydrogen, halogen, alkyl, alkoxy, amino, acylamino, —NH—CO—NH—Ar, —N═CH—N(alk)alk', nitro, cyano, phenyl, imidazolyl or SO₃H, R₃ is oxygen, NOH, NO-alk-COOK or CH—R₇, R₄ is alkyl, -alk-Het or alk-Ar, R₅ is alkyl, -alk-Ar, or C(R₄) R₅ is cycloalkyl, R₆ is hydroxy, alkyl, NR₈R₉, -alk-OH,-alk-NR₈R₉, -alk-Ar or -alk-Het, R₇ is hydroxy, alkyl, phenyl, -alk-Ar, -alk-Het, NR₁₀R₁₁ or a heterocyclic ring, R₈ and R₉ are alkyl, or R₈ is hydrogen and R₉ is hydrogen or alkyl, —COR₁₂, —CSR₃₀ or —SO₂ R₁₃, R₁₀ and R₁₁ are alkyl or cycloalkyl, R₁₂ is alkyl, cycloalkyl, phenyl, -COO-alk, —CH₂—COOX, —CH₂—NH₂, —NH-alk, —NH—Ar, —NH₂ or —NH-Het, R₁₃ is alkyl or phenyl, R₃₀ is —NH-alk, —NH—Ar, —NH₂ or —NH-Het; or R is a 2-imidazolylmethyl radical and each of R₁ and R₂ is a hydrogen atom.

(45) AMPA antagonists (I) in WO 94-26747 as shown below:

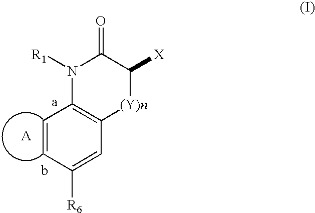

Compounds having formula (I) or a pharmaceutically acceptable salt thereof wherein R¹ is hydrogen, alkyl, or benzyl; X is O or NOR² , wherein R² is hydrogen, alkyl or benzyl; Y is N—R⁴ wherein R⁴ is hydrogen, OH or alkyl; n is 0 or 1; R⁶ is phenyl, naphthyl, thienyl, pyridyl, all of which may be substituted one or more times with substituents selected from the group consisting of halogen; CF₃, NO₂, amino, alkyl, alkoxy and phenyl; A is a ring of five to seven atoms fused with the benzo ring at the positions marked a and b.

(46) 2,3-Disubstituted-(5,6)-heteroarylfused-pyrimidine-4-ones in EP 0807 633 A2 as shown below:

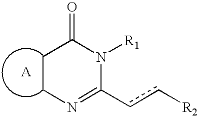

2,3-Disubstituted-(5,6)-heteroaryl fused-pyrimidine-4-ones of formula (I) and their salts are new: ring A=a group of formula (i) or (ii) both optionally substituted by H, 1-6C alkyl, halo, CF₃, (CH₂)ₙNH₂, (1-6C alkyl)amino(CH₂)ₚ, di(1-6C alkyl)amino(CH₂)ₙ, 1-6C alkoxy, 1-6C hydroxyalkyl, (l-6C alkyl)O(1-6C alkyl), CN, (1-6C alkyl)COO(1-6C alkyl), (1-6C alkyl)OCOO(1-6C alkyl), (1-6C alkyl)COO, OH, NO₂, R³CO, R⁴OCO, di(1-6C alkyl)NCO, 1-6C cycloalkyl, R⁴NHCO or phenyl (optionally substituted); A, B, D, E=C or N; F, G, J=C, N, O or S with proviso; R¹=Ph1 or pyridin-2-yl, pyridin-3-yl or pyridin-4-yl optionally substituted; Ph1=a group of formula (iii); R²=Ph2 or a group of formula (iv) or (v); K, L, M=C or N provided that only one is N; P, Q, T=C, N, O or S provided that only one can be O or S and that at least one is a heteroatom; Ph2=a group of formula (vi); R³, R⁴═H or 1-6C alkyl; R⁵═H, 1-6C alkyl, halo, CF₃, 1-6C alkoxy or 1-6C alkylthio; R⁶—R⁸═H or halo; R⁹=e.g.H, 1-6C alkyl (optionally substituted), halo, CF₃, 1-6C alkoxy (optionally substituted), 1-6C alkylthio, (CH₂)ₚOR¹³, (CH₂)ₚNH(1-6C alkyl), (CH₂)ₙN(1-6C alkyl)₂, (CH₂)ₚNH(1-5C cycloalkyl) (sic), (CH₂)ₚCONH₂, (CH₂)ₙ—CONH(1-6C allyl), (CH₂)ₚCON(1-6C alkyl)₂, (CH₂)ₚCONH(1-5C cycloalkyl) (sic), (CH₂)ₚCOOR¹³, (1-6C alkyl)OCO(1-6C alkyl), (1-6C alkyl)OCOO(1-6C alkyl), OCO(1-6C alkyl), (CH₂)ₚNHCO(1-6C alkyl) or CN; R¹⁰, R¹⁴=e.g. H, 1-6C alkyl (optionally substituted), halo, CF₃, 1-6C alkoxy (optionally substituted), 1-6C alkylthio, (CH₂)ₚOR¹³, (CH₂)ₙNH(1-6C alkyl), (CH₂)ₚN(1-6C alkyl)₂, (CH₂)ₚNH(1-5C cycloalkyl) (sic), COO(CH₂)ₚR⁴, (CH₂)ₚNH₂, 1-6C hydroxyalkyl, (1-6C alkyl)O(1-6C alkyl), CHO or CN; R¹¹, R¹²═H or halo; R¹³═H, 1-6C alkyl, CO(1-6C alkyl), COO(1-6C alkyl), CONH(1-6C alkyl) or CON(1-6C alkyl)₂; R¹⁵—R¹⁷═H, CN, 1-6C alkyl, halo, CF₃, CHO or 1-6C alkoxy; n, p=0-3; provided that when R⁹═H then one of R¹¹ and R¹² is not H.

(47) Quinoxaline compounds (I) in EP 0 511 152 A2 as shown below:

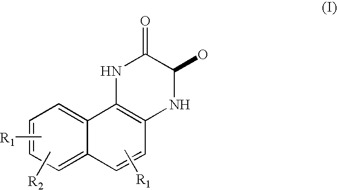

Quinoxaline compounds having the formula I wherein R¹ is H, NO₂, CN, CF₃ or halogen, R² and R³ independently are H, CN, CF₃, halogen, C(NOH) C₁₋₆ -alkyl, COR⁴ or SO₂R⁴ wherein R⁴ is C₁₋₆-alkyl-, optionally substituted, or NR⁵R⁶ wherein R⁵, R⁶ independently are H, C₃₋₆-cycloalkyl, is C₁₋₆-, optionally substituted, compositions thereof and methods of preparing the compounds are described.

(48) Hydrazone derivatives in EP 0 503 349 A1 as shown below:

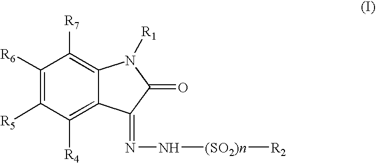

Hydrazone derivatives having the formula (I) wherein n is 0 or 1; R¹ is hydrogen, C₁₋₆-alkyl which may be branched, C₃₋₇-cycloalkyl, benzyl, phenyl which may be substituted, acyl, hydroxy, C₁₋₆-alkoxy, CH₂CO₂ R′ is hydrogen or C₁₋₆-alkyl which may be branched, CH₂CN, CH₂CONR^{IV} R^{V} wherein R^{IV} and R^{V} independently are hydrogen or C₁₋₆-alkyl, or CH₂C(═NOH)—NH₂; R² is pyridyl or phenyl, both of which may be substituted one or more times preferably into the ortho and para positions with halogen, CF₃, NO₂, CN, phenyl, SO₂NR″R′″ wherein R″ and R′″ independently are hydrogen, benzyl, or C₁₋₆-alkyl; R⁴, R⁵, R⁶, R⁷ independently are hydrogen, C₁₋₆-alkyl which may be branched, phenyl, halogen, C₁₋₆-alkoxy, NO₂, CN, CF₃, or SO₂NR¹¹R¹² wherein R¹¹ and R¹² independently are hydrogen, benzyl, or C₁₋₆-alkyl; or R⁶ and R⁷ together form an additional 4 to 8 membered carbocyclic ring which may be aromatic or partial saturated and which may be substituted with halogen, NO₂, CF₃, CN, SO₂NR¹³ R¹⁴ wherein R¹³ and R¹⁴ independently are hydrogen, benzyl, or C₁₋₆-alkyl; and R⁴ and R⁵ have the meanings set forth above; or R⁴ and R⁵ together form an additional 4 to 8 membered carbocyclic ring whihc may be aromatic or partial saturated and which may be substituted with halogen, NO₂, CF₃, CN, SO₂NR¹³R¹⁴ wherein R¹³ and R¹⁴ independently are hydrogen, benzyl, or C₁₋₆ -alkyl;, and R⁶ and R⁷ have the meanings set forth above.

(49) Dihydro-2,3-benzodiazepine derivatives (I) in EP 0 699 676 A1 as shown below:

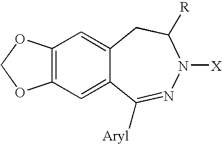

Dihydro-2,3-benzodiazepine derivatives represented by the formula I wherein R is methyl, X is acetyl and Aryl is p-nitrophenyl.

(50) Oxopyridinylquinoxaline derivatives (I) in EP 0 676 397 A1 as shown below:

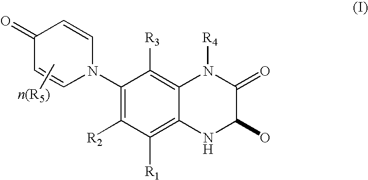

An oxopyridinylquinoxaline derivative represented by the following formula I or pharmaceutically acceptable salts thereof wherein R¹ is hydrogen, halogen, nitro or trihalomethyl; R² is hydrogen, halogen, nitro, cyano, trihalomethyl, carbamoyl, carbomoyl substituted with lower alkyl, sulfamoyl, or sulfamoyl substituted with lower alkyl; R³ is hydrogen, nitro, or halogen; R⁴ is hydrogen, lower alkyl, substituted lower alkyl, lower cycloalkyl, or substituted lower cycloalkyl; R⁵'s are substituents independently selected from the group consisting of halogen, nitro, cyano, lower alkyl, carbamoyl, and carbamoyl substituted with lower alkyl; and n is an integer of 0 to 4.

(51) Dioxo-tetrahydroquinoline derivatives (IA) in EP 0 459 561 A2 as shown below:

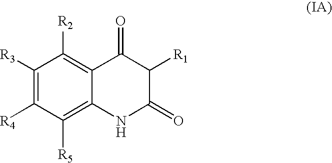

Dioxo-tetrahydroquinoline derivatives of formula (IA), wherein R¹ is a group of part formula (I) and (II); wherein U and V independently represent cyano, carboxy, —COR⁶, —CO₂R⁶, —CO₂SR⁶, —CONHOH or —CONHNH₂; n is zero or 1, preferably zero; T represents cyano, carboxy, —COR⁶, —CO₂R⁶, —CONHOH or —CONHNH₂ or a group of formula in which the broken circle represents two non-adjacent double bonds in any position in the five-membered ring; B represents a bond or a carbonyl group (C═O); W, X, Y and Z represent oxygen, sulphur, nitrogen or carbon, provided that no more than one of W, X, Y and Z represents oxygen or sulphur, at least one of W, X, Y and Z represents carbon and at least one of W, X, Y and Z is other than carbon; one of E, F and G represents nitrogen or carbon and the remainder represent carbon; A¹, A² and A³ represent one, two or three substituents not exceeding the maximum number permissible by the disposition of heteroatoms in the five- or six-membered ring, which substituents are independently selected from hydrogen, hydrocarbon, halogen, cyano, trifluoromethyl, nitro, —OR^{a}, —SR^{a}, —SOR^{a}, —SO₂ R^{a}, SO₂NR^{a}R^{b}, —NR^{a}R^{b}, —NR^{a}COR^{b}, —NR^{a}CO₂R^{b}, COR^{a}, —CO₂R^{a} or —CONR^{a}R^{b}; or A¹ and A² or A² and A³ together represent the residue of an aromatic or heteroaromatic ring; R², R³, R⁴ and R⁵ independently represent hydrogen, hydrocarbon, halogen, cyano, trifluoromethyl, nitro, —OR^{a}, —SR^{a}, —SOR^{a}, —SO₂R^{a}, SO₂NR^{a}R^{b}, —NR^{a}R^{b}, —NR^{a}COR^{b}, —NR^{a}CO₂R^{b}, COR^{a}, —CO₂R^{a} or —CONR^{a}R^{b}; or R² and R³, R³ and R⁴ or R⁴ and R⁵ together represent the residue of an aromatic or heteroaromatic ring; R⁶ represents hydrocarbon; and R^{a} and R^{b} independently represent hydrogen or hydrocarbon.

(52) Quinoxaline derivatives in EP 0 377 112 A1 as shown below:

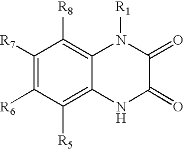

Heterocyclic dihydroxyquinoxaline compounds having the formula wherein R¹ is hydroxy, alkoxy, aryloxy, aralkyloxy, cycloalkylalkoxy, cycloalkoxy, or acyloxy; and R⁵, R⁶, R⁷ and R⁸ independently are hydrogen, NO₂, halogen, CN, SO₂NR′R′, SO₂R′, CF₃, or OR′, wherein R′ is hydrogen or C₁₋₄-alkyl.

(53) Quinoxaline derivativess in EP 0 374 534 A1 as shown below:

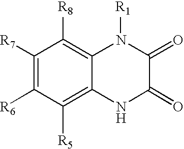

Heterocyclic dihydroxyquinoxaline compounds having the formula wherein R¹ is hydroxy, alkoxy, aryloxy, aralkyloxy, cycloalkylalkoxy, cycloalkoxy, or acyloxy; R⁵ and R⁶ together form a further fused ring, which may be substituted with hydrogen, halogen, or CN, and R⁷ and R⁸ independently are hydrogen, NO₂, halogen, CN, SO₂NR′R′, SO₂R′, CF₃, or OR′, wherein R′ is hydrogen or C₁₋₄-alkyl; or R⁷ and R⁸ together form a further fused ring, which is substituted with hydrogen, halogen, or CN, and R⁵ and R⁶ independently are hydrogen, NO₂, halogen, CN, SO₂NR′R′, SO₂R′, CF₃, or OR′, wherein R′ is hydrogen or C₁₋₄-alkyl.

(54) Quinoxaline derivatives in EP 0 315 959 A2 as shown below:

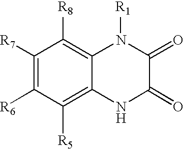

Heterocyclic dihydroxyquinoxaline compounds having the formula wherein R¹ is C₁₋₁₂-alkyl, which may optionally be substituted by hydroxy, formyl, carboxy, carboxylic esters, amides or amines, C₃₋₈ cycloalkyl, aryl, aralkyl; and wherein R⁶ is hydrogen, halogen, CN, CF₃, NO₂, or OR′, wherein R′ is C₁₋₄-alkyl and R⁵, R⁷ and R⁸ is hydrogen, provided R⁶ is not CF₃, OCH₃, NO₂, CL or Br when R¹ is CH₃; or R⁶ and R⁷ independently are NO₂, halogen, CN, CF₃, or OR′, wherein R′ is C₁₋₄-alkyl and R⁵ and R⁸ are each hydrogen; or R⁵ and R⁶ together form a further fused aromatic ring, which may be substituted with halogen, NO₂, CN, CF₃ or OR′, wherein R′ is C₁₋₄-alkyl, and R⁷ and R⁸ independently are hydrogen, halogen, CN, CF₃, NO₂ or OR′, wherein R′ is C₁₋₄-alkyl; or R⁷ and R⁸ together form a further fused aromatic ring, which may be substituted with halogen, NO₂, CN, CF₃ or OR′, wherein R′ is C₁₋₄-alkyl, and R⁵ and R⁶ independently are hydrogen, halogen, CN, CF₃, NO₂ or OR′, wherein R′ is C₁₋₄-alkyl.

(55) Heterocyclic compounds in EP 0348 872 A1 as shown below:

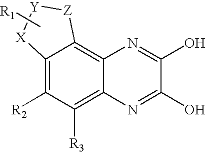

Heterocyclic dihydroxyquinaoxaline compounds having the formula wherein R¹ and R² independently are hydrogen, NO₂, NH₂, CN, halogen, SO₂NH₂; —X—Y-Z- is selected from —N═N—NR³—, —NR³—N═N—, ═N—NR³—N═, —S—CH═N—, —N═CH—S—, —CH═C(CO₂R³)—S—, —S—C(CO₂R³)═CH—, ═N—Se—N═, —N—CR³—NR³—, —NR³—CR³═N—, ═N—O—N═, —N═CR³—CR³═N—, —NH—CR³═CR³—CR³═N—, —N═CR³—CR³—CR³—NH, ═N—S—N═; wherein R³ is hydrogen, lower alkyl, CF₃.

(56) Heterocyclic dihydroxyquinoxaline derivatives in U.S. Pat. No. 4,812,458 as shown below:

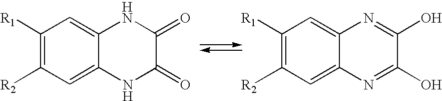

Heterocyclic dihydroxyquinoxaline compounds having the formula wherein R¹ is halogen, CN, CF₃, ethynyl, or N₃ and R² is SO₂C₁₋₃-alkyl, CF₃, NO₂, ethynyl, or CN.

(57) Pyrrolyl tetrahydrobenzoquinoxalinedione (I) in WO 96-11922 as shown below:

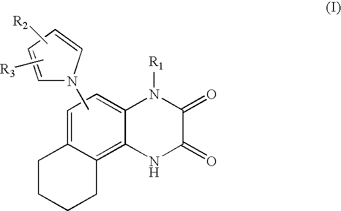

Pyrrolyl tetrahydrobenzoquinoxalinedione of formula I and their tautomeric and isomeric forms, as well as the pharmaceutically acceptable salts thereof, wherein R¹ hydrogen; an aliphatic residue with 1 to 6 C-atoms, which can carry one or two different substituents of the formula —COOR⁴, —CONHR⁴, —CO—R⁴, —OR^{4′} —NR⁴, —NH—CO—R⁴, —CONH—SO₂R⁴ or NHSO₂R⁴, of which R⁴ means hydrogen, C₁-C₄-alkyl, phenyl, benzyl, 1-phenylethyl or 2-phenylethyl, wereby the phenyl rings in R⁴ can be substituted by 1, 2 or 3 of the following substituents: C₁-C₄-alkyl, CF₃, C₁-C₄-alkoxy, F₃CO, halogen, nitro, CN, —OH, —CONHR⁵ and/or —COOR⁵ (R⁵ hydrogen, C₁-C₄-alkyl, phenyl or benzyl); —O—R⁶, of which R⁶ is hydrogen or an aliphatic residue with up to 4 C-atoms which can carry one of the following residues: —COOR⁴, —CONHR⁴, —NHCOR⁴, —NHSO₂R⁴, —OH or phenyl; R² hydrogen, C₁₋C₄-alkyl or phenyl; R³ hydrogen or the residue —(CH₂)m-R⁷, whereby m is the number 0, 1, 2, 3 or 4 and R⁷ hydrogen, C₁-C₄-alkyl, phenyl, phenylsulfonyl, NO₂, CN, —COO—(CH₂)ₙ—R⁸, —CONH—(CH₂)ₙ—R⁸, —CONHSO₂R⁴, —CO—R⁸, —CH═CH—CONHR⁸, —CH═CH—COOR⁸, —CH═NOR⁸, —CH₂—NR⁸R⁹, CH₂NH—CY—(CH₂)ₙR⁹, CH₂NH—CY—X—(CH₂)n-R⁹, CH₂NH—CO—CF₃, CH₂NH—SO₂—R⁹ wereby X and Y independently of each other are oxygen or NH, n is the number 0, 1, 2, 3 or 4, R⁸ means hydrogen or linear or branched C₁-C₄-alkyl, which can be substituted by one or two phenyl- or pyridyl-residues, and R⁹ means hydrogen, linear or branched C₁-C₆-alkyl, phenyl or pyridyl, wereby all phenyl or pyridyl residues contained in R⁸ and R⁹ can carry one or two of the following residues: O—C₁-C₄-alkyl, F, Cl, Br, J, C₁-C₄-alkyl, NO₂, CF₃, —COOR⁵, —CONHR⁵, NH₂, CN, —SO₂phenyl, —NHSO₂R⁵, —NHCOR⁵, OH, —SO₂—C₁-C₄-alkyl, —NHCOCF₃, —SO₂R⁵ and —OCF₃.

(58) Amido-quinoxalinedione (I) in WO 95-35289 as shown below:

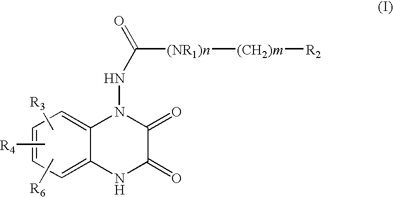

Amido-quinoxalinedione derivatives of formula (I), their tautomers, isomers and enantiomers, and their salts in which R¹═H or 1-4C alkyl; n=0-1; m=0-4; R²═H, 1-6C alkyl or phenyl (optionally mono- or di-substituted with 1-4C alkyl, OR⁶, NH₂, NO₂, NHCOR⁶, CN, CF₃, OCF₃, CO₂R⁶, F, Cl, Br, I, COR⁶ or SO₂R⁶); R3═F, Cl, Br, I, 1-4C alkyl, OR⁷, COR⁷, NH₂, NO₂, NHCOR⁷, CF₃, CN; R4, R5=H, 1-4C alkyl, 1-4C alkoxy, CF₃, OCF₃, F, Br, I, NO₂, CN or an annellated benzene ring (optionally mono or di-substituted with up to 2 1-4C alkyl, 1-4C alkoxy, CF₃, OCF₃, F, Br, I, NO₂, CN); R⁶═H, 1-4C alkyl, phenyl or benzyl; R⁷═H, 1=4C alkyl or CF₃; R⁸═H, 1-4C alkyl, phenyl, phenylsulphonyl, NO₂, CN, COO(CH₂)ᵣR, CONH(CH₂)ᵣR, COR, CH═CHCONHR, CH₂NRR′, CH₂NHCY(CH₂)ᵣR′, CH═CHCOOR, CH═NOR, CH═NR, CH2NHCY-Z(CH2)rR′, CH2NHCOCF3 or a gp. of formula (b)-(f); R9=H or 1-4C alkyl; R═H, 1-4C alkyl, phenyl, benzyl, pyridyl or benzhydryl; R′═H, 1-4C alkyl, Ph, pyridyl or 4-(R-substituted)-piperidin-1-yl; Y═O or N; Z=O or NH; r=0-4; q=0-2; the benzene rings in R⁸, R and R′ are optionally mono- or di-substituted with NH₂, OMe, OEt, Cl, Br, OCF₃, F, Me, Et, NO₂, COOR¹, CONHR¹, CH₂NHR¹, CH₂NHCOCF₃, CH₂NHCOMe, NHSO₂Me, NHCOMe or NHCOCF₃.

(59) Acid amide derivatives (I) in WO 95-31443 as shown below:

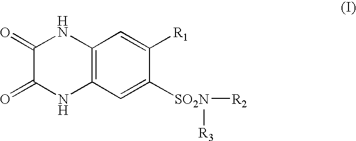

Acid amides of the formula wherein R¹ represents hydrogen or nitro, R² and R³ stand, independently from each other, for hydrogen, lower alkyl or lower alkenyl optionally carrying a substituent selected from the group consisting of halogen, hydroxy, lower alkoxy, di(lower alkyl) amino, phenyl-lower alkoxycarbonyl and a 5- to 6-membered saturated hetero-ring containing 1 or 2 nitrogen and/or oxygen atom (s); or R² and R³ form, together with the adjacent nitrogen atom, a 6-membered saturated heterocyclic group containing optionally 1 or 2 additional nitrogen atoms and/or oxygen atoms (s), said ring optionally carrying a hydroxy or a hydroxy-lower alkyl group; and all of the possible mesomers, tautomeric forms and stereoisomers of the acid amides of the formula (I) and the mixtures thereof

(60) Quinoxalindione derivatives (I) in WO 97-19066 as shown below:

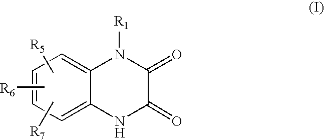

Quinoxalindione derivatives of formula (I), their isomers and salts are new: R¹═—(CH₂)ₙ—CR₂H—(CH₂)ₘ-Z; R⁵=1-6C alkyl or 2-6C alkenyl (both optionally substituted by Q), SOₚR¹³ or —CH═R¹⁵; Q=halo, OR⁸, NR⁹R¹⁰, SOₒR¹¹ or COR¹²; or aryl or heteroaryl (both optionally substituted); R⁶, R⁷═H, halo, NO₂, CN, N COR¹⁴ or OR¹⁸; or aryl or heteroaryl (both optionally substituted); 1-6C alkyl or 2-6C alkenyl (both optionally substituted by Q), SOₚR¹³ or —CH═R¹⁵; R²═H or —(CH₂)_{q}R³; R³═H, OH, 1-6C alkoxy or NR¹⁹R²⁰; n, m, q=0-3; Z=POXY, OPOXY, SO₂R²¹ COOR²², CN or tetrazolyl; R⁸, R¹⁸═H or 1-6C alkyl (optionally halo substituted); o, p=0-2; R¹¹, R¹³═H, 1-6C alkyl or optionally substituted aryl; R¹², R¹⁴, R²¹═OH, 1-6C alkoxy or NR²³R²⁴; R¹⁵═O, ═NOH or a group of formula (a): X, Y═OH, 1-6C alkoxy, 1-4C alkyl or NR²⁵R²⁶; R⁹ and R¹⁰, R¹⁶ and R¹⁷, R¹⁹ and R²⁰, R²³ and R²⁴, R²⁵ and R²⁶═H, 1-4C alkyl, aryl, or together with the N atom form a 5-7 membered saturated heterocycle (optionally containing an additional O, S or N and optionally substituted), or an unsaturated 5-membered heterocycle containing 1-3 N and optionally substituted; provided that R⁵ is not CF₃ or Me.

(61) N-substituted fused azacycloalkylquinoxalinediones (I) in WO 96-28445 as shown below:

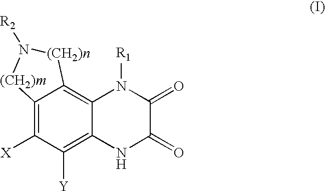

In formula (I) m and n are independently 0,1 or 2 provided that m+n is >1. R¹ is hydrogen, an alkyl or an alkylaryl; X and Y are independently hydrogen, halogen, nitro, cyano, trifluoromethyl, COOH, CONR⁴R⁵, SO₂CF₃, SO₂R⁴, SONR⁴R⁵, alkyl, alkenyl, (CH₂)_{z}CONR⁴R⁵, (CH₂)_{z}COOR⁴, or NHCOR⁴, wherein R⁴ and R⁵ are independently hydrogen, alkyl having 1 to 6 carbon atoms, cycloalkyl or alkylaryl, and z is an integer from 0 to 4; R² is alky COOR³, alkylamine, alkyquanidine, aryl, alkylaryl, COalkyl, COalkylaryl, CONR³alkyl, CONR³aryl, CONR³alkylaryl, CSNR³alkyl, CSNR³alkylaryl or a common amino acid moiety joined by an amide bond, wherein R³ is hydrogen, alkyl or alkylaryl.

(62) Spiro[heterocycle-imidazo[1,2-a]indeno[1,2-e]pyrazine]-4′-ones (1) in WO 96-14318 as shown below:

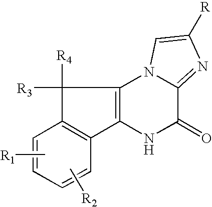

Compounds of formula (I), wherein R₃ and R₄, taken together with the carbon atom to which they are attached, form (a) a 2- or 3-pyrrolidine ring, a 2- or 4-piperidine ring or a 2-azacycloheptane ring, said rings being optionally substituted at the nitrogen atom by an alkyl radical, —CHO, —COOR₁₁, —CO-alk-COOS, —CO-alk-NR₆R₁₂ , —CO-alk-CONR₆R₈, —CO—COOR₆, —CO—CH₂—O—CH₂—COOR₆, —CO—CH₂—S—CH₂—COOR₆, —CO-alk, —CO—Ar¹¹. —CO-alk-Ar¹¹, —CO—NH—Ar¹¹, —CO—NH-alk-Ar¹¹, —CO-Het, —CO-alk-Het, —CO—NH-Het, —CO—NH-alk-Het, —CO—NH₂, —CO—NH-alk, —CO—N(alk)alk′, —CS—NH₂, —CS—NH-alk, —CS—NH—Ar¹¹, —CS—NH-Het, -alk-Het, -alk-NR₆R₈, -alk-Ar¹¹, —SO₂-alk, SO₂—Ar or —CO-cycloalkyl, where the cycloalkyl is optionally 2-substituted by a carboxy radical; or (b) a 2-pyrrolidine-5-one ring.

(63) 5H,10H-Imidazo[1,2-a]indeno[1,2-e]pyrazine-4-one derivatives (I) in WO 97-25327 as shown below:

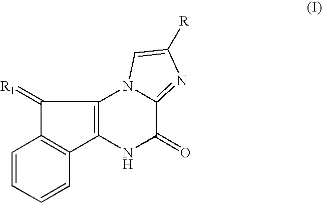

Compounds of formula (I), wherein R is a hydrogen atom or a —COOH or CH₂OH radical, R₁ is a —CH—R₂ radical, R₂ is a 3-dimethyl-1H-pyrazole-4-yl, 4-chloro-1-methylimidazole-5-yl or 3-hydroxy-isoxazole-5-yl radical except for 10-(1,3-dimethyl-1H-pyrazole-4-methylene)-5H, 10H-imidazo [1,2-a]indeno [1,2-e]pyrazine-4-one, isomers thereof, salts thereof, the preparation thereof and drugs containing said compounds.

(64) 5H,10H-Imidazo[1,2-a]indolo[3,2-e]pyrazine-4-one derivatives (I) in WO 97-25329 as shown below:

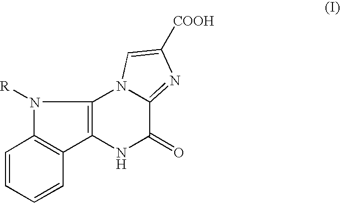

Compounds of formula (I) wherein R is a hydrogen atom or an -alk-COOH radical, racemic mixtures, enantiomers and diastereoisomers thereof, salts thereof, the preparation thereof and drugs containing said compounds.

(65) 5H,10H-Imidazo[1,2-a]indeno[1,2-e]pyrazine-4-one derivatives (I) in WO 97-25328 as shown below:

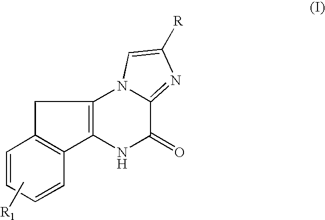

Compounds of formula (I), wherein R is a hydrogen atom or a —COOH, -alk-COOH, —PO₃H₂, CH₂—PO₃—H₂ or —CH═CH—COOH radical, or a phenyl radical substituted by a carboxy radical, R₁ is an alk-CN, -alk-COOH, alk-Het, alk-PO₃H₂ or -alk-CO—NH—SO₂R₂ radical, R₂ is an alkyl or phenyl radical, alk is an alkyl radical, Het is a saturated or unsaturated mono- or polycyclic heterocyclic ring containing 1-9 carbon atoms and one or more heteroatoms selected from O, S and N, said heterocyclic ring optionally being substituted by one or more alkyl, phenyl or phenylalkyl radicals, with the proviso that when R is a hydrogen atom or a —COOH or —PO₃H₂ radical, R₁ cannot be -alk-COOH, isomers, racemic, mixtures, enantiomers and diastereoisomers thereof, salts thereof, the preparation thereof, intermediates thereof and drugs containing said compounds.

(66) 2-Substituted 5H,10H-imidazo[1,2-a]indeno[1,2-e]pyrazine-4-ones (I) in WO 97-25326 as shown below:

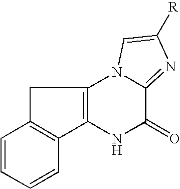

Compounds of formula (I), wherein R is a —CO—CH₂—PO₃H₂, —CO—NH-tetrazole-5-yl, —CO—NHOH, CO—NH—NH₂, -alk-COOH, -alk-COOalk′, —CH₂—PO₃H₂, —CO—NH—SO₂—R₁ or —CH═CH—COOH radical, or a phenyl radical substituted by a carboxy radical, alk and alk′ are an alkyl radical and R₁ is an alkyl, trifluoromethyl or phenyl radical optionally substituted by a carboxy or alkoxy-carbonyl radical, racemic mixtures, isomers, enantiomers and diastereoisomers thereof, salts thereof, the preparation thereof and drugs containing said compounds.

(67) Indeno[1,2-e]pyrazine-4-ones (I) in WO 97-10246 as shown below:

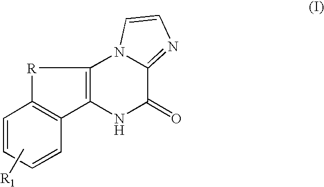

Compounds of formula (I), wherein R is a C═CH—R₂, C(R₃)R₄, CH—NH₂, CH—CH(OH)—COOH or CH—CH(OH)—COOalk radical, R₁ is an alk-NH₂ or alk-NH—CO—R₅ radical, R₂ is a —COOH or —COOalk radical, R₃ is an alkyl, -alk-Ar or -alk-Het radical, R₄ is an NH₂, —NH-alk, —N(alk)-alk′, —NH—CO-alk, —NH—CO—Ar′, —NH—CO-ALK-Ar′, —NH—CO-Het, —NH—CO-alk-Het, —NH—CO-alk-COOH, —NH—CO-alk-COOalk′, -alk-COOH, -alk-COOalk′, —NH—CO—NH₂, —NH—CO—NH-alk or —NH—CO—NH—Ar′ or —NH—CO—NH-alk-Ar′ radical, or R₃ and R₄, together with the carbon atom to which they are attached, form a 2- or 3-pyrrolidine, 2- or 4-piperidine or 2-azacycloheptane substituted or unsubstituted ring, R₅ is an —NH₂, —NH-alk, —NH—Ar′, —NH-cycloalkyl, —NH-alk-Ar′ or —N(alk)-alk′ radical, the salts thereof, the preparation thereof and medicaments containing same.

(68) 5H,10H-Imidazo[1,2-a]indeno[1,2-e]pyrazine-4-one derivatives (I) in WO 96-31511 as shown below:

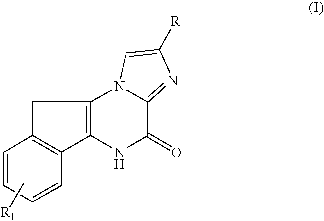

Compounds of formula (I), wherein R is a hydrogen atom or a carboxy, alkoxycarbonyl, —CO—NR₄R₅, —PO₃H₂ or —CH₂OH radical and R₁ is an alk-NH₂, -alk-NH—CO—R₃, -alk-COOR₄, -alk-CO—NR₅R₆ or —CO—NH—R₇ radical.

(69) Decahydroisoquinoline compounds (I) in U.S. Pat. No. 5,356,902 as shown below:

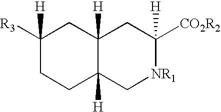

Compound of the formula (I) wherein: R¹ is hydrogen, C₁₋₁₀ alkyl, arylalkyl, alkoxycarbonyl, aryloxycarbonyl or acyl; R² is hydrogen, C₁-C₆ alkyl, substituted alkyl cycloalkyl, or arylalkyl; R³ is a group of the formula; R⁴ is hydrogen, C₁₋₄ alkyl, CF₃, phenyl, bromo, iodo, or chloro; or a pharmaceutically acceptable salt thereof.

(70) Phosphonoalkylquinolin-2-ones in U.S. Pat. No. 5,342,946 as shown below:

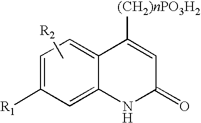

Having the general formula: wherein n is 0, 1, 2 or 3; R1 and R2 are selected from the group consisting of hydrogen, halogen, halomethyl, nitro, amino, alkoxy, hydroxyl, hydroxymethyl, C1 to C6 lower alkyl and C7 to C12 higher alkyl, aryl, and aralkyl; and the pharmaceutically acceptable salts thereof.

(71) Imidazobenzodiazepine compounds (I) in U.S. Pat. No. 5,270,306 as shown below:

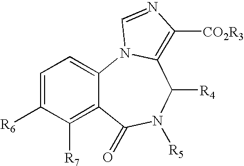

Compound having the formula wherein R³ is hydrogen, C₁₋₈-alkyl which may be branched, or cycloalkylmethyl; R⁷ and R⁸ are independently hydrogen, halogen, CF₃, CN, NO₂, NH₂, C₁₋₄-alkyl or C₁₋₄-alkoxy; and R⁴ is hydrogen and R⁵ is hydrogen or C₁₋₇ alkyl; or R⁴and R⁵ together signify (CH₂)ₙ wherein n is an integer of 2-3.

(72) Isatine derivatives in U.S. Pat. No. 5,192,792 as shown below:

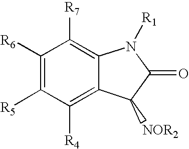

Indole-2,3-dione-3-oxime compound having the formula wherein R¹ is hydrogen, C₁₋₆-alkyl which may be branched, C₃₋₇-cyclo-alkyl, benzyl, phenyl which may be substituted, acyl, hydroxy, C₁₋₆-alkoxy, CH₂CO₂R″ wherein R′ is hydrogen or C₁₋₆-alkyl which may be branched, CH₂CN, CH₂CONR^{IV} R^{V} wherein R^{IV} and R^{V} independently are hydrogen or C₁₋₆-alkyl, or CH₂C(═NOH)NH₂; R² is (1) alkenyl of from two to six carbon atoms, preferably allyl, (2) alkynyl of from two to six carbons, preferably propargyl, (3) (CH₂)₁₋₆CO₂H, (4) (CH₂)₁₋₆CONHR wherein R is C₁₋₆ alkyl, optionally branched; aryl which is phenyl optionally substituted by one or more of lower alkyl of from one to four carbons, halogen wherein halogen is fluoro, chloro, bromo, or iodo, trifluromenthyl, cyano, carboxy, alkoxycarbonyl wherein the alkoxy is of from one to four carbons, alkylthio wherein the alkyl is of from one to four carbons, nitro, acyl of from two to four carbons, hydroxy, C₁₋₆-alkoxy, CH₂CO₂R′ wherein R′ is hydrogen or C₁₋₆-alkyl which may be branched, CH₂CN, CH₂CONR^{IV} R^{V} wherein R^{IV} and R^{V} independently are hydrogen or C₁₋₆ alkyl, optionally branched; aralkyl which is aryl as defined above attached through C₁₋₄ alkyl, or SO₂R¹⁰ wherein R¹⁰ is C₁₋₆ alkyl, optionally branched; aryl which is phenyl optionally substituted by one or more of lower alkyl of from one to four carbons, halogen wherein halogen is fluoro, chloro, bromo, or iodo, tri-fluoromethyl, cyano, carboxy, alkoxycarbonyl wherein the alkoxy is of from one to four carbons, alkylthio wherein the alkyl is of from one to four carbons nitro, acyl of from two to four carbons, hydroxy, C₁₋₆ alkoxy, CH₂CO₂ R′ wherein R′ is hydrogen or C₁₋₆-alkyl which may be branched, CH₂CN, CH₂CONR^{IV} R^{V} wherein R^{IV} and R^{V} independently are hydrogen or C₁₋₆ alkyl, optionally branched; aralkyl which is aryl as defined above attached through C₁₋₄ alkyl; R₄, R₅, R₆, R₇ independently are hydrogen, C₁₋₆ alkyl, which may be branched, phenyl, halogen, C₁₋₆-alkoxy, NO₂, CN, CF₃, or SO2NR′″R′″ wherein R″ and R″″ independently are hydrogen, or C₁₋₆-alkyl; or R⁶ and R⁷ together form an additional 4 to 7 membered ring which may be aromatic or partial saturated and which may be substituted with halogen, NO₂, CF₃, CN, SR₂NR′″R″″ wherein R″ and R″″ independently are hydrogen, or C₁₋₆-alkyl; and R⁴ and R⁵ have the meanings set forth above.

(73) Aryl-spaced decahydroisoquinoline-3-carboxylic acids in U.S. Pat. No. 5,446,051:

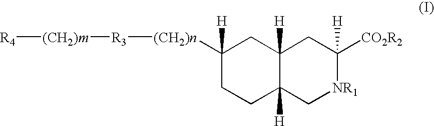

Preferably, the compounds are of the general formula (I) wherein R¹ is H, C₁₋-C₁₀-alkyl, arylalkyl, alkoxycarbonyl, aryloxycarbonyl, or acyl; R₂ is H, C₁-C₆-alkyl, substituted alkyl, C₄-C₇ cycloalkyl or arylalkyl; R₃ is aryl, arylalkyl, heterocycle, substituted heterocycle, C₄-C₇ cycloalkyl or C₄-C₇ cycloalkenyl; R₄ is CO₂H, SO₃H, PO₃H₂, or one of the following cyclic compounds: wherein R⁵ is H, C₁₋₆-alkyl or aryl; m=0, 1 or 2; and n=0, 1 or 2; or a pharmaceutically acceptable salt thereof.

(74) Quinoxalindione derivatives reported in WO 94-25469 and shown below:

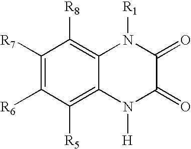

Quinoxalindione derivatives represented by the above formula wherein R1 is (CH₂)ₙ—CR2H—(CH₂)ₘ-Z and R5, R6, R7 and R8 together or independently are hydrogen, C1-C6 alkyl, CF₃, nitro, halogen, NR9R10, cyano, SOₚR11, SO₂NR12R13, SO₃H, SO₃C₁₋₆-alkyl or OR14; R2 is hydrogen, or (CH₂)_{q}—R3; R3 is hydrogen, OH, C₁₋₆-alkoxy or NR15R16, and n, m and q are 0,1,2, or 3; Z is POXY, OPOXY, OR17, NR18R19, NH—COR20, NH—SO₂R21, SO₂R22, CO₂R23, halogen, cyano or tetrazole; R11 is hydrogen, C1-C6 alkyl, phenyl; p is 0, 1, or 2; R12, R13, R17 or R23 is hydrogen or C1-C4 alkyl; R14 is hydrogen or 1-3 halogen substituted C1-C6 alkyl; R20 and R21 are C1-C6 alkyl or halogen substituted phenyl or hetaryl; R22 is OH, C1-C6 alkoxy or NR24R25; X and Y are together or independently OH, C1-C6 alkoxy, C1-C4 alkyl or NR18R19; R9 and R10 are together or independently hydrogen, CO—C1-C6 alkyl, phenyl or C1-C6 alkyl, which may be substituted with C1-C4 alkoxy or C1-C4 alkyl mono- or disubstituted NH₂ group, or together with the nitrogen form a 5-7 membered heterocyclic ring which may contain additional N, S or O and can be substituted, or form five membered heterocyclic ring which may contain 1-3 nitrogens and can be substituted; R15 and R16, R18 and R19 together or independently are hydrogen, C1-C4 alkyl, phenyl or together with the oxygen form 5-7 membered heterocyclic ring which may contain additional N, S or O and can be substituted, or form five membered heterocyclic ring which may contain 1-3 nitrogens and can be substituted; R24 and R25 together or independently are hydrogen, C1-C4 alkyl, or together with the oxygen form 5-7 membered heterocyclic ring which may contain additional N, S or O, and their isomers and salts and provided R2 is hydrogen and Z POXY or CO₂R23 then R5-R8 is not hydrogen; and provided R2 is hydrogen, Z POXY or CO₂R23 and R5, R6, R7 and R8 are CF₃, NO₂, halogen, NH₂ or methyl, the compounds of the above formula are double-substituted and provided R1 is methanophosphonic acid and R6 cyano or substituted imidazole then together R5, R7 and R8 is not hydrogen and provided R1 is methanosulphonic acid and R6 is CF₃ or NO₂ and R7 is imidazole, R5 and R8 is not hydrogen; and provided R1 is CH₂—COOH and R5 and R8 is hydrogen, R6 and R7 is not halogen or methyl; and the pharmaceutically acceptable salts thereof

(75) Isoquinolinyl-carboxylic acid compounds reported in U.S. Pat. No. 5,606,062 and shown below:

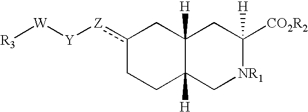

Isoquinolinyl-carboxylic acid compounds represented by the above formula wherein R1 is hydrogen, C1-C10 alkyl, arylalkyl, alkoxycarbonyl, or acyl; R2 is hydrogen, C1-C6 alkyl, substituted alkyl, cycloalkyl, or arylalkyl; R3 is CO₂H, SO₃H, CONHSO₂R8, or a group of formula: 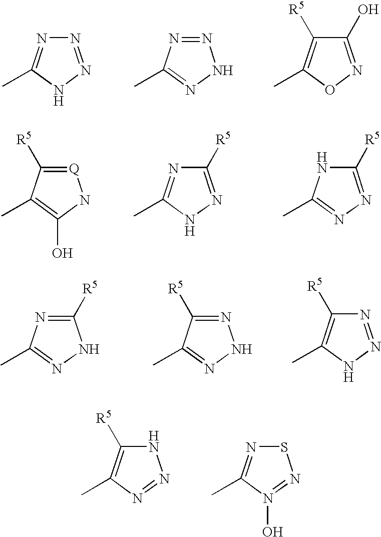
W is (CH₂)ₙ, S, SO, SO₂; Y is CHR7, NR4, O, S, SO, or SO₂; Z is NR6, CHR7, or CH; or W and Y together are HC═CH or C═C, or Y and Z together are HC═CH or C═C; R4 is hydrogen, C1-C4 alkyl, phenyl, or acyl; R5 is hydrogen, C1-C4 alkyl, CF₃, phenyl, hydroxy, amino, bromo, iodo, or chloro; R6 is acyl; R7 is independently hydrogen, C1-C4 alkyl, phenyl, or substituted phenyl; R8 is C1-C4 alkyl or tetrazole-5-yl; and n is 0, 1, or 2; provided that when Y is NR4, O, S, SO, or SO₂, W is (CH₂)ₙ and Z is CHR7 or CH; further provided that when W is S, SO, or SO₂, Y is CHR7, Z is CHR7 or CH or Y and Z together are HC═CH or C═C; further provided that when W and Z are CH₂, Y is not S; further provided that when W and Y together are HC═CH or C═C, Z is CHR7; and the pharmaceutically acceptable salts thereof.

(76) Decahydroisoquinoline compounds described in U.S. Pat. No. 5,527,810 as shown below:

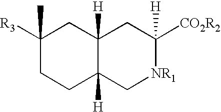

Decahydroisoquinoline represented by the above formula wherein R1 is hydrogen, C1-C10 alkyl, arylalkyl, alkoxycarbonyl, aryloxycarbonyl or acyl; R2 is hydrogen, C1-C6 alkyl, substituted alkyl, cycloalkyl, or arylalkyl; R3 is a group of the formula: 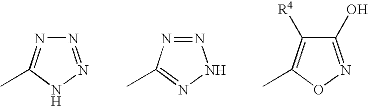
R4 is hydrogen, C1-C4 alkyl, CF₃, phenyl, bromo, iodo, or chloro, and the pharmaceutically acceptable salts thereof

(77) Cycloalkynoxalinediones shown in U.S. Pat. No. 5,721,234 as exemplified below:

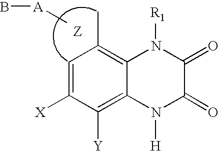

Cycloalkynoxalinediones represented by the above formula wherein Z is an alicyclic fused ring having 5 to 7 carbon atoms; R1 is hydrogen, an alkyl or an arylalkyl; X and Y are independently hydrogen, halogen, nitro, cyano, COOH, CONR2R3, SONR2R3 wherein R2 and R3 are independently hydrogen, alkyl having 1 to 6 carbon atoms, cycloalkyl or aralkyl; and A is O, CH₂, NR4, CH₂NR4, CN, tetrazole or CO wherein R4 is hydrogen, alkyl, hydroxyalkyl, aminoalkylamine or aralkyl, wherein (i) when A is O, CH₂, NR4, or CH₂NR4 then B is hydrogen, alkyl, alkenyl, aryl, aralkyl, hydroxyalkyl, alkoxy, aminoalkyl, heterocyclic, alkylheterocyclic, heterocyclic-methyl, heterocyclic-ethyl, alkylcarbonyl, cycloalkylcarbonyl, arylcarbonyl, aralkylcarbonyl, heterocyclic-carbonyl, alkylheterocyclic-carbonyl, any of which may be unsubstituted or substituted by one or more hydroxy, CO₂H, mercapto, amino, alkyl or butoxycarbonyl group, CONR5R6 wherein R5 is hydrogen, alkyl having 1 to 6 carbon atoms, or aralkyl, and R6 is alkyl, aryl, or aralkyl, or N, R5, and R6 taken together form a cyclic amine, or when A is NR4 or CH₂NR4 then B is a common amino acid moiety joined by an amide bond or B joins with R4 and the nitrogen to form a four to seven membered heterocyclic ring, provided that when Z is a fused cyclohexyl ring and R4 is hydrogen then B is not hydrogen; (ii) when A is CN then B is not present and Z is not a fused cyclohexyl ring; (iii) when A is tetrazole then B is hydrogen or alkyl having 1 to 6 carbon atoms; and (iv) when A is CO then B is hydroxy, alkoxy, aralkoxy, alkyl having 1 to 6 carbon atoms, aralkyl, NR7R8 wherein R7 is hydrogen, alkyl having 1 to 6 carbon atoms, or aralkyl, and R8 is alkyl, aryl, or aralkyl, or N, R7, and R8 taken together from a cyclic amine, and the pharmaceutically acceptable salts thereof

(78) Phosphonoalkylquinolin-2-ones as reported in U.S. Pat. No. 5,510,338 and shown below:

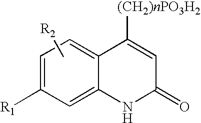

Phosphonoalkylquinolin-2-ones represented by the above formula wherein n is 0, 1, 2 or 3. R1 or R2 are selected from the group consisting of hydrogen, halogen, halomethyl, nitro, amino, alkoxy, hydroxyl, hydroxymethyl, C1 to C6 lover alkyl and C7 to C12 higher alkyl, aryl, and aralkyl; and the pharmaceutically acceptable salts thereof.

(79) 2,3-Benzodiazepine derivatives (I) and (II) in P 97 00688 as shown below:

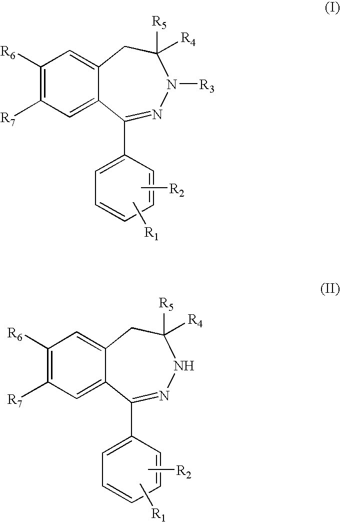

2,3-Benzodiazepine derivatives and medicinal preparations containing such drugs represented by the formula I wherein R1 and R2 can be, independently from each other, hydrogen, halogen, alkyl group with 1-4 carbonic atoms, alcoxy group with 1-4 carbonic atoms, nitro group, trifluoromethyl group, or group having a general structure of —NR8R9, where the meaning of R8 and R9, can be, independently from each other, hydrogen, alkyl group with 1-4 carbonic atoms, or group having a general structure of —COR₁₀, where R10 means hydrogen atom, alkyl group with 1-6 carbonic atoms substituted in given cases, aryl group with 6-10 carbonic atoms, alcoxy group with 1-4 carbonic atoms, cycloalkyl group with 3-5 carbonic atoms, alkenyl group with 2-6 carbonic atoms, cycloalcoxy group with 3-5 carbonic atoms, or group having a general structure of —NR11R12, where the meaning of R11 and R12, independently from each other, hydrogen atom, alkyl group with 1-4 carbonic atoms, cycloalkyl group with 3-5 carbonic atoms, or aryl group with 6-10 carbonic atoms, the meaning of R3 can be alkyl group with 1-4 carbonic atoms, cycloalkyl group with 3-5 carbonic atoms, or group having a general structure of —CO—R13, where the meaning of R13 can be the same as given for R10, the meaning of R4 and R5, can be, independently from each other, hydrogen atom, or alkyl group with 1-3 carbonic atoms, the meaning of R6 and R7, can be, independently from each other, hydrogen atom, Cl atom, or Br atom, with the condition that if any of R4 or R5 means hydrogen atom, the second can only be other than hydrogen atom, further, the isomers, salts obtained with acid addition, and the medicinal preparations originating from them.

2,3-Benzodiazepine derivatives represented by the formula II wherein R1, R2, R4, R5, R6 and R7 is given for general structure (I).

(80) Oxadiazole derivatives (I) in DE 196 43 037 A1 as shown below:

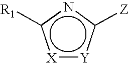

Oxadiazole derivatives of formula (I), and their racemates, enantiomers, diastereomers, mixtures and acid addition salts, are new. One of X, Y═N and the other ═O; Z=pyridyl substituted by S1, or Ar (optionally substituted by R² and R³); Ar=phenyl substituted at the 2-position by S1 and optionally at the 6-position by S2; or Ar=phenyl substituted at the 3- or 4-position by S2; S1=B-V-D-R4, B—N(D-R⁴)D-R⁴¹ or a group of formula (a) (optionally substituted by halo, oxo, OR7, OCOR7, 1-4C alkyl, 2-6C alkenyl or 2-6C alkynyl); S2=B-V-D-R⁴ or B—N(D-R⁴)D-R⁴¹; V, E=O, S or NR⁷; D=1-10C alkylene, 2-10C alkenylene or 2-10C alkynylene (all optionally substituted by Q1); B=bond or as for D; n, m=1-3, and n+m is at least 2; R¹=1-10C alkyl, 2-10C alkenyl or 2-10C alkynyl (all optionally substituted by one or more Q2), a norbornane, norbornene, di(3-6C)cycloalkyl-methyl, adamantane or noradamantane residue (all optionally substituted by 1-4C alkyl), H, phenyl (optionally substituted by 1-3 Q3 (directly or via 1-4C alkylene)), phenyl (substituted by B—N(D-R⁴)DR⁴¹, B-V-D-R⁴, OCH₂O or OCH₂CH₂O), A″-A″, 3-7C cycloalkyl (optionally substituted by Q2), fluorenyl, a [3.3.0]bicyclooctane group; or an optionally substituted group of formula (b)-(d); y=1 or 2; z=0-2; R², R³=1-10C alkyl, 2-10C alkenyl or 2-10C alkynyl (all optionally substituted by Q2), SH, NR⁵R⁶, halo, NO₂, CF₃, OR⁷, SR⁷, COOR⁷, 6-10C aryl, aryl(1-6C)alkyl or 6-10C aryloxy; or R²+R³ complete an unsaturated fused 5-7 membered ring (optionally containing one or more heteroatoms, and optionally substituted by OR⁷, NR⁵R⁶, halo, CN, NO₂, CF₃, COOR⁷, 1-10C alkyl, 2-10C alkenyl or 2-10C alkynyl; R⁴, R⁴¹=1-10C alkoxy, 2-10C alkenyloxy or 2-10C alkynyloxy (all optionally substituted by Q2), OH, halo, NO₂, CF₃, CN, SH, 1-6C alkylmercapto, A-Ar′, OAr′, Ar′-substituted 1-6C alkoxy, M′, NR⁵R⁶ or 3-8C cycloalkoxy (optionally substituted by oxo, OR⁷ or OCOR⁷); R⁵, R⁶=1-10C alkyl, 2-10C alkenyl or 2-10C alkynyl (all optionally substituted by OH, optionally substituted phenyl, optionally substituted benzyl, NR⁷R⁷¹ or 1-8C alkoxy), H, optionally substituted 3-6C cycloalkyl or 6-10C aryl (optionally substituted by halo, OR⁷, 1-4C alkyl, NR⁷R⁷¹, SO₃H or COOR⁷); or NR⁵R⁶=an optionally unsaturated 5-6 membered ring, optionally containing other heteroatoms, and optionally substituted by Q4; R⁷, R⁷¹═H, R, 2-4C alkenyl, 2-4C alkynyl, or benzyl or phenyl (both optionally substituted by OH, Cl, Br or OMe); R⁸=1-4C alkyl, 2-4C alkenyl, 2-4C alkynyl, phenyl, benzyl or 3-6C cycloalkyl; R⁹═H, 1-4C alkyl, COOR⁷, CH₂OR⁷, CONR⁵R⁶ or phenyl; Q1=CN, CHO, COOR⁷, CONHSO₂R⁷, CONR⁵R⁶, CH═NOR⁷, COR⁸, CH(OR⁷)R⁸, CH(OR⁷)OR⁷¹, CH═CHR⁹, NR⁵R⁶, NHCOR⁷, NHCONR⁵R⁶, NHCOOR⁷, OR⁷, OCOR⁷, OCOOR⁷, OCONR⁵R⁶, SR⁷, SOR⁷, SO₂R⁷, SO₃H, SO₂NR⁵R⁶, halo, 1,3-dioxolan or 1,3-dioxan); Q2=oxo or Q1; A″=1-6C-alkyl, 2-6C alkenyl or 2-6C alkynyl; A=H or as for A″; A′=phenyl (optionally ring substituted, directly or via a 1-4C alkylene bridge, by one or more groups Q3), 3-7C cycloalkyl (optionally ring substituted, directly or via a 1-4C alkylene bridge, by one or more groups Q2), M, CONHM or NHCOM; Ar′=aryl substituted by one or more Q3; M′=5-7 membered heterocycle linked via C, containing one or more heteroatoms, optionally substituted by benzyl, 1-4C alkyl, halo, OR⁷, CN, NO₂, NH₂, CH₂NR⁵R⁶, OH, oxo, ketal, ethylene ketal, COOH, SO₃H, COOR⁷, CONR⁵R⁶, COR⁸, SO₂R⁷ or CONR⁵R⁶ (sic)); M=heterocycle as for M′, (which may also be linked by N, and also be substituted by optionally substituted phenyl or substituted benzyl); Q3=halo, 1-4C alkyl, CF₃, CHO, COOR⁷, CONHSO₂R⁷, CONR⁵R⁶, CH═NOR⁷, COR⁸, CH(OH)R⁸, CH(OR⁷)OR⁷¹, CH═CHR⁹, NR⁵R⁶, NO₂, 1-4C alkyl-NR⁵R⁶, NHCOR⁷, NHCONR⁵R⁶, NHCOOR⁷, NH—SO₂R⁷, OR⁷, OCOR⁷, OCONR⁵R⁶, SR⁷, SOR⁷, SO₂R⁷, SO₃H or SO₂NR⁵R⁶; Q4=1-4C alkyl, (CH₂)n-Q5, halo, OR⁷, CN, NO₂, NR⁷R⁷, SO₃H, COOR⁷, CONR⁷R⁷¹, SO₂R⁷, oxo or a ketal; Q5=phenyl, NH₂, 1-4C alkylamino, di(1-8C)alkylamino or NHCOOR⁷; heteroatoms=N, O, S.

(81) Quinoxalindione derivatives reported in WO 96-37500 and shown below:

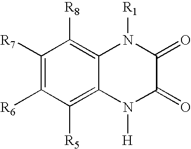

Quinoxalindione derivatives represented by the formula I wherein R1 is —(CH₂)ₙCR²H—(CH₂)ₘ-Z and R⁵, R⁶, R⁷ and R⁸ together or independently are hydrogen, C₁₋₆-alkyl in which one or more hydrogen atoms are replaced with halogen atoms, nitro, halogen, NR⁹R¹⁰, cyano, SOₚS¹¹, SO₂NR¹²R¹³, SO₃H, SO₃C₁₋₆-alkyl or OR¹⁴; R² hydrogen or (CH₂)_{q}—R³; R3 hyrdogen, hydroxy, C₁₋₆-alkoxy or NR¹⁵R¹⁶; n, m and q can be 0, 1, 2 or 3; Z is POXY, OPOXY, SO₂R¹⁷, COR¹⁸, halogen, cyano or tetrazole; R¹¹ H, C₁₋₆-alkyl, phenyl; p 0, 1 or 2; R¹² and R¹³ are independently hydrogen or C₁₋₄-alkyl; R¹⁴ A-R¹⁹, or means C₆₋₁₂-aryl- or hetaryl, which can be substituted with halogen, C₁₋₆-alkoxy, hydroxy, cyano, NR²⁰R²¹, eventually with halogen substituted C₁₋₆-alkyl and/or COR²² and A linear or branched, saturated or unsaturated alkyls with C₁₋₂₀-carbon atoms in which one or several carbons can be substituted by O, S and/or NR²⁶ and can be substituted with halogen; and R¹⁹ hydrogen, NR²⁴R²⁵, halogen, C₁₋₆-alkyl, which eventually is substituted with halogen, C₁₋₆-alkoxy, COR²³, CN or one C₆₋₁₂-aryl or hetaryl which is substituted with halogen, and/or substituted COR²²; and R¹⁸ hydrogen, C₁₋₄-alkyl, hydroxy, C₁₋₆-alkoxy or NR²⁷R²⁸; R¹⁷, R²² and R²³ hydroxy, C₁₋₆-alkoxy or NR²⁹R³⁰, R²⁶ hydrogen, C₁₋₆-alkyl, C₁₋₆-alkenyl, X and Y are similar or different and are hydroxy, C₁₋₆-alkoxy, C₁₋₄-alkyl or NR²⁷R²⁸; R⁹ and R¹⁰, R²⁰ and R²¹ and/or R²⁵ and R²⁴, are similar or different and hydrogen, CO—C₁₋₆-alkyl, phenyl or C₁₋₆-alkyl, which with C₁₋₄-alkoxy or one eventually with C₁₋₄-alkyl mono- or di-substituted amino group substituted is, or together with nitrogen atom bild 5-7-membered saturated heterocyclic ring, which may contain additional N, S- or O-atom and can be substituted, or bild 5-membered saturated heterocyclic ring, which contains 1-3 N atoms and can be substituted; R¹⁵ and R¹⁶, R²⁷ and R²⁸, R²⁹ and R³⁰ are similar or different and are hydrogen, C1-4-alkyl, phenyl or bild together with nitrogen atom 5-7-membered saturated heterocyclic ring, which may contain additional O-, S-, N-atom and can be substituted or bild 5-membered saturated heterocyclic ring, which can contain 1-3 nitrogen atoms and can be substituted, although R⁵—R⁸ always mean OR¹⁴, and R¹⁴ does not mean H or eventuall 1-3 halogen substituted C₁₋₆-alkyl.

AMPA and/or Kainate Receptor Channel Blocker

The inhibitors of the present invention also include AMPA and/or kainate receptor channel blockers. The term “AMPA and/or kainate receptor channel blockers” is used to refer to moieties that reduce the permeability of channels associated with the AMPA and/or kainate receptor to cations (preferably to Na⁺,K⁺ and/or Ca²⁺ ions). AMPA and/or kainate receptor channel blockers can therefore be used to prevent a signal being transmitted due to ionic flux that would otherwise occur when glutamate binds to the AMPA and/or kainate receptor.

AMPA and/or kainate receptor channel inhibitors include e.g. fluorowillardiine and Joro spider toxin.

Having described the inhibitors of the present invention, their therapeutic uses will now be discussed in greater detail.

Therapeutic Uses

Inhibitors of the present invention may be used in human and veterinary medicine. Treatments may be prophylactic or may be in respect of existing conditions.

As explained supra, the inhibitors may be used in the manufacture of a medicament for treating a demyelinating disorder. The term “demyelinating disorder” is used herein to include any disorder that results in a reduced level of myelination.

Demylinating disorders include acute disseminated encephalomyelitis, acute demyelinating polyneuropathy (Guillain Barre syndrome), chronic inflammatory demyelinating polyneuropathy, multiple sclerosis, Marchifava-Bignami disease, central pontine myelinolysis, Devic syndrome, Balo disease, HIV- and HTLV-myelopathy, and progressive multifocal leucoencephalopathy.

Demylinating disorders also include secondary demyelinating disorders—i.e. where bystander myelin loss occurs as a consequence of a secondary pathological insult.

Examples of secondary demyelinating disorders are CNS lupus erythematodes, polyarteriitis nodosa, Sjögren syndrome, sarcoidosis and isolated cerebral vasulitis.

The present invention includes within its scope pharmaceutically acceptable compositions useful in treating demyelinating disorders which comprise an inhibitor of the present invention. The inhibitor will usually be provided in combination with a pharmaceutically acceptable carrier. It may be used in any suitable form, provided that it can still act in inhibiting the interaction of glutamate with the AMPA and/or kainate receptor complex. For example, pharmaceutically acceptable salts, esters, hydrates, etc. may often be used.

Pharmaceutical compositions within the scope of the present invention may include one or more of the following: preserving agents, solubilising agents, stabilising agents, wetting agents, emulsifiers, sweeteners, colorants, odourants, salts, buffers, coating agents or antioxidants.

They may contain a further therapeutically active agent in addition to an inhibitor of the present invention. The further therapeutically active agent may be an immunosuppresive agent (e.g. corticotrophin, a glucocorticoid, cyclophosphamide, cyclosporine, azothioprine or mitozantrone), an interferon (IFN; IFN-beta-1a e.g. Rebif and Avonex; IFN-beta-1b e.g. Betaseron and Betaferon; IFN-alpha-2a e.g. Alphaferone; IFN-alpha-2b e.g. Viraferon), a phosphodiesterase type IV inhibitor, a humanised monoclonal antibody against a leukocyte adhesion molecule (e.g. Antegran), a synthetic polypeptide (e.g. glatiramer acetate, copolymer-1) a tissue matrix metalloproteinase (MMP) inhibitor (e.g. hydroxamnic acid-based inhibitors of MMPs) or a tumour necrosis factor (TNF) inhibitor (e.g. Thalidomide or TNF-receptor immunoglobulin fusion protein).

The combination of an inhibitor of the present invention and a further therapeutically active agent may be used simultaneously, seperately or sequentially to treat a demyelinating disorder. It may provide synergistically effective combination. The further therapeutically active agent may be an immunosuppresive agent (e.g. corticotrophin, a glucocorticoid, cyclophosphamide, cyclosporine, azothioprine or mitozantrone), an interferon (IFN; IFN-beta-1a e.g. Rebif and Avonex; IFN-beta-1b e.g. Betaseron and Betaferon; IFN-alpha-2a e.g. Alphaferone; IFN-alpha-2b e.g. Viraferon), a phosphodiesterase type IV inhibitor, a humanised monoclonal antibody against a leukocyte adhesion molecule (e.g. Antegran), a synthetic polypeptide (e.g. glatiramer acetate, copolymer-1) a tissue matrix metalloproteinase (MMP) inhibitor (e.g. hydroxamic acid-based inhibitors of MMPs) or a tumour necrosis factor (TNF) inhibitor (e.g. Thalidomide or TNF-receptor immunoglobulin fusion protein).

A pharmaceutical composition within the scope of the present invention may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) routes. Such a composition may be prepared by any method known in the art of pharmacy, for example by admixing one or more active ingredients with a suitable carrier. Preferably it will be provided in unit dosage form. It will normally be provided in a sealed, sterile container e.g. in an an ampoule, a vial, a bottle, a blister pack, etc.

Different drug delivery systems can be used to administer pharmaceutical compositions of the present invention, depending upon the desired route of administration. Such systems include tablets, capsules, lozenges, pastilles, powders, solutions, suspensions, syrups, ointments, pastes, oils, aerosols, suppositories, enemas, pessaries, tampons, sprays, nebulizers, injectable compositions, etc.

Dosages of the inhibitors of the present invention can vary between wide limits, depending upon the nature of the treatment and the age and condition of the individual to be treated. However, a daily dosage of from 0.5 mg to 1000 mg, preferably of from 50-200 mg may be suitable. The dosage may be repeated as often as appropriate. If side-effects develop, the amount and/or frequency of the dosage can be reduced, in accordance with good clinical practice.

The therapeutic uses of the present invention are based upon animal models that are discussed in the examples and that are believed to be reliable. Prior to the present invention there was no disclosure of the use of antagonists of the present invention for treating demyelinating disorders. Only limited characterisation studies of kainate and AMPA receptors had been performed. Matute et al had preformed various studies. For example in PNAS 95, 10229-10234, 1998 (which was published after the earlier priority date of the present application) studies acute and chronic kainate excitotoxic damage to the optic nerve are reported.

The present invention will now be described by way of example only, with reference to the accompanying drawings, wherein:

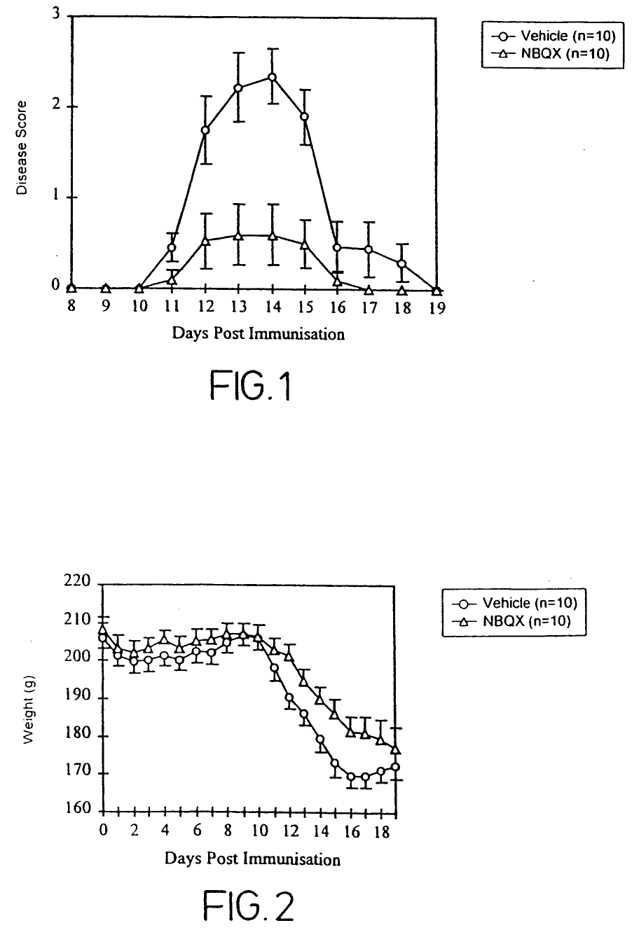

FIG. 1 shows that the AMPA receptor antagonist NBQX reduces severity of paralysis during EAE in rats. NBQX (30 mg/kg i.p. twice daily; 10-16 dpi) significantly reduces the peak disease score. Data represent the mean±SEM of disease score (n=10/group).

FIG. 2 shows that NBQX (30 mg/kg i.p. twice daily; 10-16 dpi) reduces weight loss during the course of EAE in rats prior to cessation of treatment (16 dpi). Data represent the mean±SEM of disease score (n=10/group).

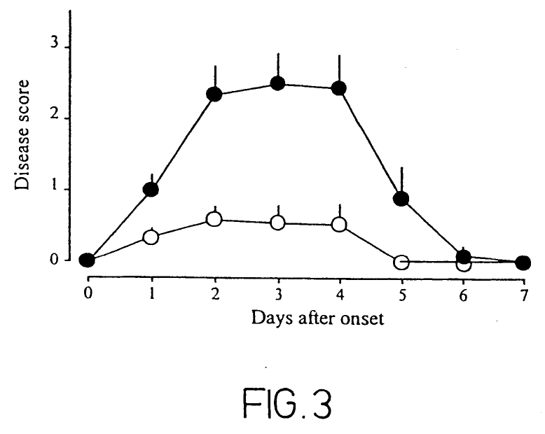

FIG. 3 shows that the non-competitive AMPA antagonist GYKI53773 reduces the severity of paralysis during EAE. GYKI53773 (30 mg/kg i.p. twice daily; 10-16 dpi) significantly reduces the peak disease score. Data represents the mean±sem of disease score, standardised to days after disease onset (● Vehicle n=9; ◯ GYKI53773 n=10).

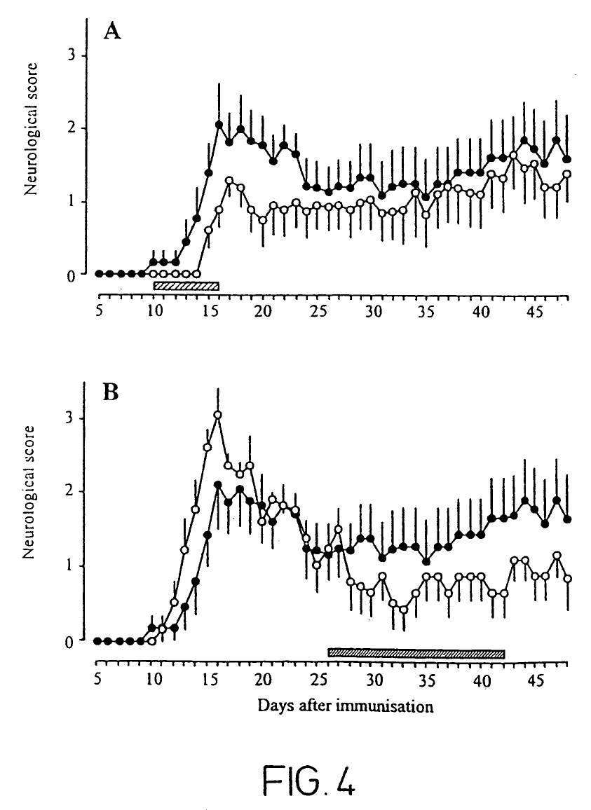

FIG. 4 shows that the AMPA receptor antagonist NBQX reduces the severity of paralysis during chronic EAE. in A, NBQX, 30 mg/kg (◯; n=10) and vehicle (●; n=9) were administered i.p. twice daily for 7 days starting on day 10 post immunisation (10-16 dpi; stippled bar). In B, NBQX, 30 mg/kg kg (◯; n=7) and vehicle (●; n=10) were administered i.p. once daily for 17 days commenced on dpi 26 (26-42 dpi; hatched bar). Data represents die mean±sem of disease score.

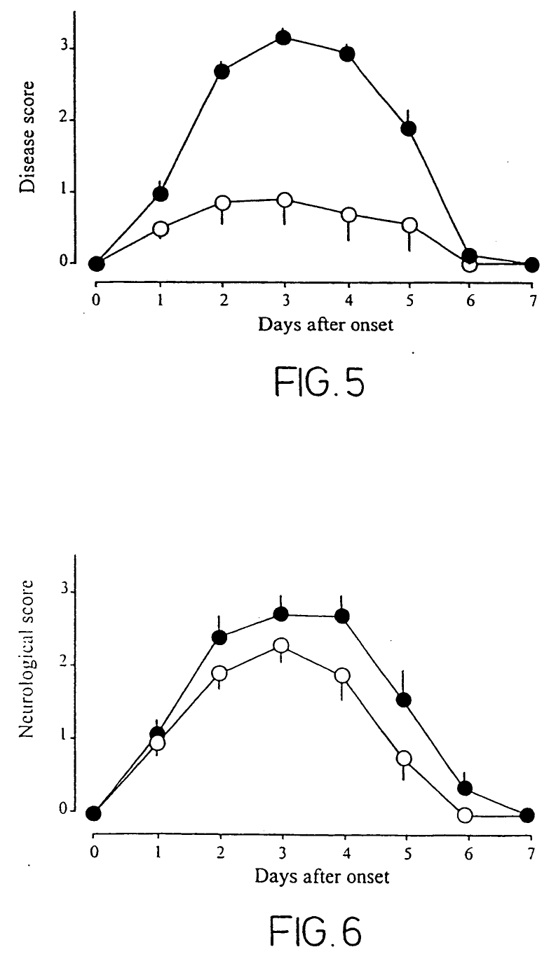

FIG. 5 shows that the AMPA/kainate receptor antagonist MPQX reduces the severity of paralysis during EAE. MPQX (10 mg/kg i.p. twice daily; 10-16 dpi) significantly reduces the peak disease score. Data represents the mean±sem of disease score, standardised to days after disease onset (● Vehicle n=26; ◯ MPQX n=12).

FIG. 6 shows that the non-competitive AMPA antagonist GYKI52466 reduces the severity of paralysis during EAE. GYKI52466 (30 mg/kg i.p. twice daily; 10-16 dpi) significantly reduces the peak disease score. Data represents the mean±sem of disease score, standardised to days after disease onset (● Vehicle n=15; ◯ GYKI52466 n=16).

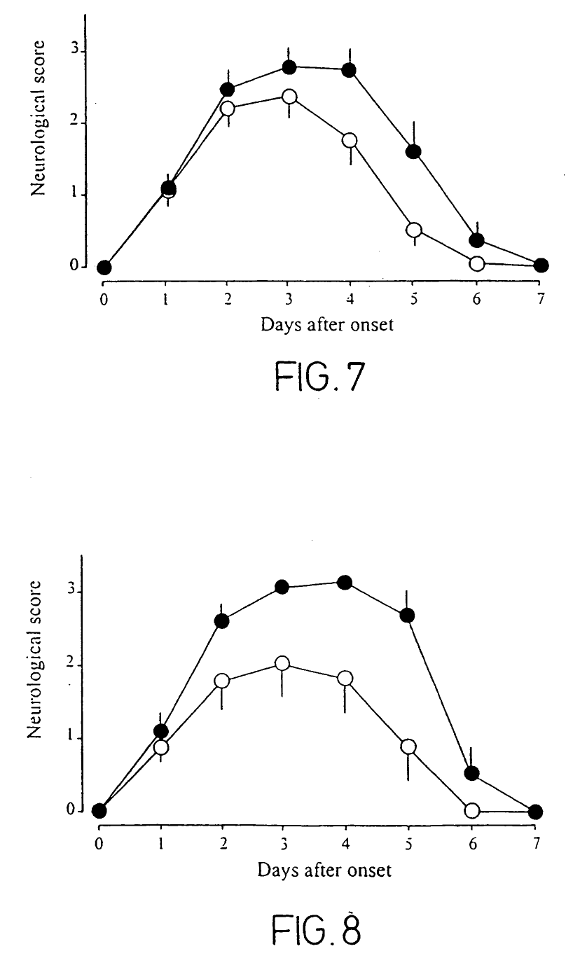

FIG. 7 shows that the non-competitive AMPA antagonist BIIR561 redcues the severity of paralysis during EAE. BIIR561 (30 mg/kg i.p. twice daily; 10-16 dpi) significantly reduces the peak disease score. Data represents the mean±sem of disease score, standardised to days after disease onset (● Vehicle n=15; ◯ BIIR561 n=16).

FIG. 8 shows that the non-competitive AMPA antagonist CP465022 reduces the severity of paralysis during EAE. CP465022 (10 mg/kg i.p. twice daily; 10-16 dpi) significantly reduces the peak disease score. Data represents the mean±sem of disease score, standardised to days after disease onset (● Vehicle n=9; ◯ CP465022 n=9).

### EXAMPLES

Experimental allergic encephalomyelitis (EAE), an inducible autoimmune disease, represents the best characterized animal model of a demyelinating disorder and drugs active in this model proved to be active in humans (Pender M P (1996). Experimental autoimmune encephalomyclitis, In Autoimmune Neurological Disease, Editors Pender M P and McCombe P A, Cambridge University Press. pp 26-88).

Here we describe a surprising observation on the reduction in neurological deficits during acute EAE in rats following treatment with a non-immunomodulatory and non-antiinflamatory agent, the AMPA receptor antagonists, 2,3-dihydroxy6-nitro-7-sulfamoylbenzo-(F)-quinoxaline (NBQX). Furthermore, the non-competitive AMPA antagonists (−)1-(4-aminophenyl)-4-methyl-7,8-methylenedioxy-4,5-dihydro-3-methylcarbamoyl-2,3-benzodiazepine (GYKI53773), 1-(-aminophenyl)-4-methyl-7,8-methylene-dioxy-5H-2,3-benzodiazepine (GYKI52466), 5-(2-[N,N-dimethylamino]oxy-phenyl)-3-phenyl-1,2,4-oxadiazol (BIIR561) and 3-(2-chlorophenyl)-2-[2-(6-[(diethylamino)methyl]-2-pyridinyl]ethenyl]-6-fluoro- 4(3H)-quinazolinone (CP465022), and the AMPA/kainate receptor antagonist [1,2,3,4-tetrahydro-7-morpholinyl-2,3-dioxo-6-(trifluoromethyl)quinoxalin-1-yl]methylphosphonate (MPQX) reduced neurological deficits during acute EAE. In addition we also describe the reduction in neurological deficit during chronic EAE in mice following treatment with NBQX.

Animals

Female Lewis rats (205+10 g) obtained from Charles River, Kent, UK, were housed in pairs under environmentally controlled conditions (6:00 a.m.-6:00 p.m. light/dark cycle; 22-24° C.; 45-55% humidity) and allowed free access to food and water. Experimental groups consisted of 10 animals. Female Biozzi mice (20±5 g) obtained from Harlan, UK, were housed under the conditions described above. Experimental groups consisted of 7-10 animals.

Induction of Acute-Active EAE in Lewis Rats

Rats were immunised in each hind foot with 50 μl of inoculum containing 50 μg guinea pig myelin basic protein (UP, prepared by the method of Dunkley and Carnegie (1974); final concentration 2 mg/ml), emulsified in Freund's complete adjuvant (CFA; Sigma, UK) containing Mycobacterium tuberculosis H37Ra (final concentration 5.5 mg/ml; Difco Laboratories, UK).

Assessment of Clinical EAE in Lewis rats

Animals were weighed and monitored daily and clinical disease scored as (0) no clinical signs; (1) flaccid tail and weight loss; (2) hind limb hypotonia with further weight loss; (3) complete hind limb paralysis; (4) paraplegia and (5) death. In addition, intermediate scores were assigned to animals which showed a loss of tonicity in the distal half of the tail.(score=0.5), paralysis of one hind limb (score=2.5) or complete hind limb paralysis with forelimb weakness (score=3.5). During the period of compound administration (10-16 days post immunisation; dpi) animals were scored 15 h after injection of vehicle or NBQX to avoid any acute effect of treatment on disease score.

Induction of Chronic-Active EAE in Biozzi Mice

Spinal cords from Biozzi mice (Ab/H, H-2^{dql}) were homogenised and freeze dried. Lyophilised spinal cord homogenate was reconstituted in phosphate buffered saline to a final concentration of 6.6 mg/ml. Incomplete Freund's adjuvant (IFA, Difco) was supplemented with M. tuberculosis (H37Ra, Difco) and M. butyricum (8:1). Biozzi mice were immunised subcutaneously on day 0 and day 7 in the flank at three sites with 0.3 ml of the emulsion (1 mg spinal cord homogenate, 60 μg of combined M. tuberculosis and butyricum). In addition, mice were injected i.p. with 200 ng of pertussis toxin (Bordetella pertussis, Calbiochem; 2 g/ml in phosphate buffered saline) immediately and 24 h after immunisation with neuroantigens.

Assessment of Clinical EAE in Biozzi mice

Monitoring of neurological deficits was performed daily by blinded observer before administration of vehicle or drugs. The following scoring system was used to grade neurological impairment: (0) no detectable changes; (1) flaccid tail; (2) impairment of righting reflex and/or loss of muscle tone; (3) complete hind limb paralysis; (4) paraplegia; and (5) death. During the period of compound administration (10-16 dpi or 26-42 dpi) animals were scored 15 h after injection of vehicle of NBQX to avoid any acute effect of treatment on disease score.

NBQX Administration Regime

NBQX was initially dissolved in NaOH and diluted with water. pH was adjusted with HCl. Rats were injected i.p. twice daily (9 am. and 5 p.m.) on days 10 to 16 post immunisation with either vehicle or NBQX in the dose of 30 mg/kg. Mice were injected i.p. either twice daily (9 am. and 5 p.m.) on days 10 to 16 post immunisation or once daily (9 a.m.) on days 26 to 42 post immunisation with either vehicle or NBQX in the dose of 30 mg/kg.

GYKI53773 Administration Regime

GYKI53773 was suspended in 5% cremophore in saline. Rats were injected i.p. twice daily (9 a.m. and 5 p.m.) on days 10 to 16 post immunisation with either vehicle or GYKI53773 in the dose of 30 mg/kg.

GYKI52466 Administration Regime

GYKI52466 was suspended in 5% cremophore in water. Rats were injected i.p. twice daily (9 a.m. and 5 p.m.) on days 10 to 16 post immunisation with either vehicle or GYKI52466 in the dose of 30 mg/kg.

BIIR561 Administration Regime

BIIR561 was suspended in 5% cremophore in water. Rats were injected i.p. twice daily (9 a.m. and 5 p.m.) on days 10 to 16 post immunisation with either vehicle or BIIR561 in the dose of 30 mg/kg.

CP465022 Administration Regime

CP465022 was suspended in 5% cremophore in water. Rats were injected i.p. twice daily (9 a.m. and 5 p.m.) on days 10 to 16 post immunisation with either vehicle or CP465022 in the dose of 10 mg/kg.

MPQX Administration Regime

MPQX was initially dissolved in NaOH and diluted with water. pH was adjusted with HCl. Rats were injected i.p. twice daily (9 a.m. and 5 p.m.) on days 10 to 16 post immunisation with either vehicle or MPQX in the dose of 10 mg/kg.

Results

Effect of NBQX on Disease Progression During EAE in the Lewis Rat

Following immunisation with MBP, neurological deficit developed in 10/10 vehicle treated animals, 8 of which displayed paralysis of one or both hind limbs; the mean disease onset and duration were 11.8 dpi and 4.7 dpi respectively (FIG. 1 and Table 1). Twice daily treatment from day 10 to 16 post immunisation with NBQX completely prevented the development of paralysis in 6 out of 10 rats, whilst one animal exhibited loss of tone in the most proximal part of the tail (score 0.25) for one day only. The remaining 3 rats displayed paresis of score 1, 2.5 and 3, the onset and duration of which were similar to vehicle injected animals. Thus NBQX significantly reduced disease duration (p<0.001), and peak and cumulative disease score (p<0.01) relative to vehicle treatment. NBQX also conferred protection on weight loss, significantly delaying the onset until 13 dpi (p<0.01) and decreasing the percent body weight lost at the cessation of NBQX administration (day 16; FIG. 2 and Table 1).

TABLE 1
Parameters of disease activity during Lewis rat acute EAE

| | Incidence | ^{a}Onset | Duration | Peak Disease | ^{b}Cumulative | ^{c}Weight |
|---|---|---|---|---|---|---|
| Treatment | (%) | (d.p.i.) | (days) | Score | Disease Score | Loss (%) |
| Vehicle | 10/10 (100) | 11.8 (11-14) | 4.7 (4-5) | 2.7 (2-3.25) | 9.8 (5.5-13) | 18 (12-23) |
| NBQX | 4/10 (75) | 11.8 (11-12) | 1.5 (0-5)†† | 0.7 (0-3)† | 2.4 (0-11.5)† | 14 (5-20)* |

Values in the table represent the mean and range where n = 10;
*p < 0.05, p < 0.01 and p < 0.001 vs vehicle, Student t-test or Mann-Whitney U-test for parametric and non-parametric data respectively.
^{a}n = 4 for NBQX.
^{b}Cumulative disease score calculated by summation of individual daily disease scores.
^{c}Calculated as the weight on cessation of treatment (16 dpi) expressed as a percent of the maximum weight before disease onset.

Effect of GYKI53773 on Disease Progression During EAE in the Lewis Rat

Following immunisation with MBP, neurological deficit developed in 8/9 vehicle treated animals; the mean disease onset and duration were 11.9 dpi and 3.8 days respectively (FIG. 3 and Table 2). Twice daily treatment from day 10 to 16 post immunisation with GYKI53773 significantly reduced disease duration (p<0.05) and peak and cumulative disease score (p<0.01) relative to vehicle treatment.

TABLE 2
Parameters of disease activity during Lewis rat acute EAE.

| | Incidence | ^{a}Onset | Duration | Peak Disease | ^{b}Cumulative | ^{c}Weight |
|---|---|---|---|---|---|---|
| Treatment | (%) | (d.p.i.) | (days) | Score | Disease Score | Loss (%) |
| Vehicle | 8/9 (89) | 11.9 (10-16) | 3.8 (0-5) | 2.6 (0-3.5) | 9.5 (0-14.75) | 19 (7-26) |
| GYKI53773 | 6/10 (60) | 12.7 (11-14) | 1.9 (0-5)* | 0.9 (0-2.25)† | 2.1 (0-6)† | 16 (11-20) |

Values in the table represent the mean and range where n = 10;
*p < 0.05, and
†p < 0.01 vs vehicle, Student t-test or Mann-Whitney U-test for parametric and non-parametric data respectively.
^{a}n = 6 for GYKI53773.
^{b}Cumulative disease score calculated by summation of individual daily disease scores.
^{c}Calculated as the weight on cessation of experiment (20 dpi) expressed as a percent of the maximum weight before disease onset.

Effect of NBQX on Disease Progression During Chronic EAE in the Biozzi Mouse

To determine whether AMPA receptor antagonists affect the clinical outcome of chronic EAE, NBQX was administered i.p. to immunised mice. Treatment with NBQX, 30 mg/kg twice daily for 7 days starting on dpi 10, improved neurological outcome reducing disease severity between dpi 10 to 48 [F(1,38)=9.21, P<0.001] (FIG. 4A). Treatment with NBQX, 30 mg/kg once daily for 17 days commencing on dpi 26 also reduced disease severity between dpi 28 to 48 [F(1,20)=2.76, P<0.05] (FIG. 4B).

Effect of MPQX on Disease Progression During EAE in the Lewis Rat

Following immunisation with MBP, neurological deficit developed in 26/26 vehicle treated animals; the mean disease onset and duration were 11.2 dpi and 4.8 days respectively (FIG. 5 and Table 3). Twice daily treatment from day 10 to 16 post immunisation with MPQX significantly delayed disease onset (p<0.05), reduced disease duration (p<0.001) and peak and cumulative disease score (p<0.001) relative to vehicle treatment.

TABLE 3
Parameters of disease activity during Lewis rat acute EAE.

| | Incidence | ^{a}Onset | Duration | Peak Disease | ^{b}Cumulative | ^{c}Weight |
|---|---|---|---|---|---|---|
| Treatment | (%) | (d.p.i.) | (days) | Score | Disease Score | Loss (%) |
| Vehicle | 26/26 (100) | 11.2 (10-13) | 4.8 (3-6) | 3.5 (2.5-4) | 11.0 (8-14.5) | 21 (15-28) |
| MPQX | 7/12 (58) | 12.3 (11-14)* | 1.7 (0-5)†† | 1.2 (0-3)†† | 3.2 (0-11.5)†† | 22 (13-30) |

Values in the table represent the mean and range where n = 10;
*p < 0.05 and
††p < 0.001 vs vehicle, Student t-test or Mann-Whitney U-test for parametric and non-parametric data respectively.
^{a}n = 7 for MPQX.
^{b}Cumulative disease score calculated by summation of individual daily disease scores.
^{c}Calculated as the weight on cessation of experiment (18 dpi) expressed as a percent of the maximum weight before disease onset.

Effect of GYKI52466 on Disease Progression During EAE in the Lewis Rat

Following immunisation with MBP, neurological deficit developed in 14/15 vehicle treated animals; the mean disease onset and duration were 11.4 dpi and 4.3 days respectively (FIG. 6 and Table 4). Twice daily treatment from day 10 to 16 post immunisation with GYKI52466 significantly reduced peak and cumulative disease score (p<0.01) relative to vehicle treatment.

TABLE 4
Parameters of disease activity during Lewis rat acute EAE.

| | Incidence | ^{a}Onset | Duration | Peak Disease | ^{b}Cumulative | ^{c}Weight |
|---|---|---|---|---|---|---|
| Treatment | (%) | (d.p.i.) | (days) | Score | Disease Score | Loss (%) |
| Vehicle | 14/15 (93) | 11.4 (10-16) | 4.3 (0-6) | 2.8 (0-3.5) | 10.9 (0-14.75) | 20 (7-26) |
| GYKI52466 | 15/16 (60) | 11.6 (10-13) | 4.0 (0.6) | 2.4 (0-3.0)** | 8.0 (0-13.75)** | 20 (8-26) |

Values in the table represent the mean and range where n = 10;
**p < 0.01 vs vehicle, Mann-Whitney U-test for non-parametric data.
^{a}n = 15 for GYKI2466.
^{b}Cumulative disease score calculated by summation of individual daily disease scores.
^{c}Calculated as the weight on cessation of experiment expressed as a percent of the maximum weight before disease onset.

Effect of BIIR561 on Disease Progression During EAE in the Lewis Rat

Following immunisation with MBP, neurological deficit developed in 14/15 vehicle treated animals; the mean disease onset and duration were 11.4 dpi and 4.3 days respectively (FIG. 7 and Table 5). Twice daily treatment from day 10 to 16 post immunisation with BIIR561 significantly reduced peak (p<0.05) and cumulative disease score (p<0.001) relative to vehicle treatment.

TABLE 5
Parameters of disease activity during Lewis rat acute EAE.

| | Incidence | ^{a}Onset | Duration | Peak Disease | ^{b}Cumulative | ^{c}Weight |
|---|---|---|---|---|---|---|
| Treatment | (%) | (d.p.i.) | (days) | Score | Disease Score | Loss (%) |
| Vehicle | 14/15 (93) | 11.4 (10-16) | 4.3 (0-6) | 3.5 (0-3.5) | 10.9 (0-14.75) | 20 (7-26) |
| BIIR561 | 16/16 (100) | 12.4 (11-19) | 3.9 (1-5)†† | 2.6 (0.5-3.25)* | 7.8 (0.5-11.5)† | 17 (5-23)* |

Values in the table represent the mean and range where n = 10;
*p < 0.05 and
†p < 0.001 vs vehicle, Student t-test or Mann-Whitney U-test for parametric and non-parametric data respectively.
^{a}n = 16 for BIIR561.
^{b}Cumulative disease score calculated by summation of individual daily disease scores.
^{c}Calculated as the weight on cessation of experiment expressed as a percent of the maximum weight before disease onset.

Effect of CP465022 on Disease Progression During EAE in the Lewis Rat

Following immunisation with MBP, neurological deficit developed in 9/9 vehicle treated animals; the mean disease onset and duration were 10.6 dpi and 5.1 days respectively (FIG. 8 and Table 6). Twice daily treatment from day 10 to 16 post immunisation with CP465022 significantly delayed disease onset (p<0.01), reduced disease duration (p<0.05), peak and cumulative disease score (p<0.01) relative to vehicle treatment.

TABLE 6
Parameters of disease activity during Lewis rat acute EAE.

| | | | | Peak | | |
|---|---|---|---|---|---|---|
| | Incidence | ^{a}Onset | Duration | Disease | ^{b}Cumulative | ^{c}Weight |
| Treatment | (%) | (d.p.i.) | (days) | Score | Disease Score | Loss (%) |
| Vehicle | 9/9 (100) | 10.6 (10-16) | 5.1 (4-6) | 3.5 (3.0-3.5) | 12.8 (11.75-14.25) | 23 (18-26) |
| CP465022 | 8/9 (89) | 13.4 (11-17)** | 3.4 (0-5)* | 2.0 (0-3.0)** | 7.2 (0-13.5)** | 20 (10-28) |

Values in the table represent the mean and range where n = 10;
*p < 0.05 and
**p < 0.01 vs vehicle, Student t-test and Mann-Whitney U-test for parametric and non-parametric data respectively.
^{a}n = 8 for CP465022.
^{b}Cumulative disease score calculated by summation of individual daily disease scores.
^{c}Calculated as the weight on cessation of experiment expressed as a percent of the maximum weight before disease onset.

### GENERAL REMARKS

The foregoing description of the invention is merely illustrative thereof and it should therefore be appreciated that various variations and modifications can be made without departing from the spirit or scope of the invention as set forth in the accompanying claims.

Where preferred or optional features are described in connection with particular aspects of the present invention, they shall be deemed to apply mutatis mutandis to other aspects of the invention unless the context indicates otherwise.

All documents cited herein are hereby incorporated by reference, as are any citations referred to in said documents.

1. A pharmaceutical composition for treating a demyelinating disorder comprising an acetyl-aminophenyl-dihydro-methyl-dioxolo-benzodiazepine and a pharmaceutically acceptable carrier.

2. The pharmaceutical composition of claim 1, wherein the acetyl-aminophenyl-dihydro-methyl-dioxolo-benzodiazepine is 1-(4-aminophenyl)-4-methyl-7,8-methylene-dioxy-5H-2,3-benzodiazepine (GYKI52466) or (−)1-(4-aminophenyl)-4-methyl-7,8-methylenedioxy-4,5-dihydro-3-methylcarbamoyl-2,3-benzodiazepine (GYKI53773).

3. The pharmaceutical composition of claim 1, wherein the acetyl-aminophenyl-dihydro-methyl-dioxolo-benzodiazepine is 1-(4-aminophenyl)-4-methyl-7,8-methylene-dioxy-5H-2,3-benzodiazepine (GYKI52466).

4. The pharmaceutical composition of claim 1, wherein the acetyl-aminophenyl-dihydro-methyl-dioxolo-benzodiazepine is (−) 1-(4-aminophenyl)-4-methyl-7,8-methylenedioxy-4,5-dihydro-3-methylcarbamoyl-2,3-benzodiazepine (GYKI53773).

5. The pharmaceutical composition of claim 1, wherein the demyelinating disorder is acute disseminated encephalomyelitis, acute demyelinating polyneuropathy (Guillain Barre syndrome), chronic inflammatory demyelinating polyneuropathy, multiple sclerosis, Marchifava-Bignami disease, central pontine myelinolysis, Devic syndrome, Balo disease, HIV- or HTLV-myelopathy, progressive multifocal leucoencephalopathy or a secondary demyelinating disorder.

6. The pharmaceutical composition of claim 1, wherein the demyelinating disorder is acute disseminated encephalomyelitis, multiple sclerosis, Devic syndrome, Balo disease or a secondary demyelinating disorder.

7. The pharmaceutical composition of claim 6, wherein the secondary demyelinating disorder is CNS lupus erythematodes, polyarteriitis nodosa, Sjögren syndrome, sarcoidosis or isolated cerebral vasulitis.

8. A method of treating a demyelinating disorder comprising administering an effective amount of an acetyl-aminophenyl-dihydro-methyl-dioxolo-benzodiazepine.

9. The method of claim 8, wherein the acetyl-aminophenyl-dihydro-methyl-dioxolo-benzodiazepine is 1-(4-aminophenyl)-4-methyl-7,8-methylene-dioxy-5H-2,3-benzodiazepine (GYKI52466) or (−) 1-(4-aminophenyl)-4-methyl-7,8-methylenedioxy-4,5-dihydro-3-methylcarbamoyl-2,3-benzodiazepine (GYKI53773).

10. The method of claim 8, wherein the acetyl-aminophenyl-dihydro-methyl-dioxolo-benzodiazepine is 1-(4-aminophenyl)-4-methyl-7,8-methylene-dioxy-5H-2,3-benzodiazepine (GYKI52466).

11. The method of claim 8, wherein the acetyl-aminophenyl-dihydro-methyl-dioxolo-benzodiazepine is 1-(4-aminophenyl)-4-methyl-7,8-methylenedioxy-4,5-dihydro-3-methylcarbamoyl-2,3-benzodiazepine (GYKI53773).

12. The method of claim 8, wherein the demyelinating disorder is acute disseminated encephalomyelitis, acute demyelinating polyneuropathy (Guillain Barre syndrome), chronic inflammatory demyelinating polyneuropathy, multiple sclerosis, Marchifava-Bignami disease, central pontine myelinolysis, Devic syndrome, Balo disease, HIV- or HTLV-myelopathy, progressive multifocal leucoencephalopathy or a secondary demyelinating disorder.

13. The method of claim 8, wherein the demyelinating disorder is acute disseminated encephalomyelitis, multiple sclerosis, Devic syndrome, Balo disease or a secondary demyelinating disorder.

14. The method of claim 13, wherein the secondary demyelinating disorder is CNS lupus erythematodes, polyarteriitis nodosa, Sjögren syndrome, sarcoidosis or isolated cerebral vasulitis.

15. A pharmaceutical composition for treating a demyelinating disorder comprising an acetyl-aminophenyl-dihydro-methyl-dioxolo-benzodiazepine and a pharmaceutically acceptable carrier combined with one or more agents selected from the group consisting of an immunosuppresive agent, an interferon(IFN), a phosphodiesterase type IV inhibitor, a humanized monoclonal antibody against a leukocyte adhesion molecule, a synthetic polypeptide, a tissue matrix metalloproteinase (MMP) inhibitor, and a tumour necrosis factor (TNF) inhibitor.

16. A method of treating a demyelinating disorder comprising administering a combination of an effective amount of an acetyl-aminophenyl-dihydro-methyl-dioxolo-benzodiazepine with one or more agents selected from the group consisting of an immunosuppresive agent, an interferon (IFN), a phosphodiesterase type IV inhibitor, a humanised monoclonal antibody against a leukocyte adhesion molecule, a synthetic polypeptide, a tissue matrix metalloproteinase (MMP) inhibitor, and a tumour necrosis factor (TNF) inhibitor.

17. The method of claim 16, wherein said combination is administered simultaneously, separately or sequentially.
